SEQUENCE LISTING (50 sequences)

SEQ ID NO: 1 - protein, 366 aa, Leptospira meyeri
MNETGSIGIIETKYAEFKELILNNGSVLSPVVIAYETYGTLSSSKNNAILICHALSGDAHAAGYHSGSDKKPGWWDDYIGPGKSFDTNQYFIICSNVIGGCKGSSGPLSIHPETSTPYGSRFPFVSIQDMVKAQKLLVESLGIEKLFCVAGGSMGGMQALEWSIAYPNSLSNCIVMASTAEHSAMQIAFNEVGRQAILSDPNWKNGLYDENSPRKGLALARMVGHITYLSDDKMREKFGRNPPRGNILSTDFAVGSYLIYQGESFVDRFDANSYIYVTKALDHYSLGKGKELTAALSNATCRFLVVSYSSDWLYPPAQSREIVKSLEAADKRVFYVELQSGEGHDSFLLKNPKQIEILKGFLENPN

SEQ ID NO: 2 - protein, 377 aa, Corynebacterium glutamicum
MPTLAPSGQLEIQAIGDVSTEAGAIIKNAEIAYHRWGEYRVDKEGRSNVVLIEHALTGDSNAADWWADLLGPGKAINTDIYCVICTNVIGGCNGSTGPGSMHPDGNFWGNRFPATSIRDQVNAEKQFLDALGITTVAAVLGGSMGGARTLEWAAMYPEIVGAAAVLAVSARASAWQIGIQSAQIKAIENDHHWHEGNYYESGCNPATGLGAARRIAHLTYRGELEIDERFGTKAQKNENPLGPYRKPDQRFAVESYLDYQADKLVQRFDAGSYVLLTDALNRHDIGRDRGGLNKALESIKVPVLVAGVDTDILYLYHQQEHLSRNLGNLLAMAKIVSPVGHDAFLTESRQMDRIVRNFFSLISPDENNPSTYIEFYI

SEQ ID NO: 3 - protein, 336 aa, Deinococcus geothermalis
MTALISQPDLLPPPAPERCPPQQTARLFRETPLLLDCGQVVQDVRVAYHTYGTPSDHAILVLHALTGTSAVHEWWPDFLGEGKPLDPTRDYIVCANVLGGCAGSTGPAELPRVNGEDPPLTLRDMARVGRALLEELGVRRVSVIGASMGGMLAYAWLLECPDLVDRAVIIGAPARHSPWAIGLNTAARNAIRAAPGGEGLKVARQIAMLSYRSPESFALTQSGWGTRRPGTPDITTYLEHQGEKLSTRFCERSYLALTGAMDRFQPTDAELRSIRVPVLVVGISSDVLYPPAEVRTYAGLLPRGQYLELQSPHGHDAFLIDPQGLPEAAAAFLHGA

SEQ ID NO: 4 - protein, 368 aa, Sulfurimonas autotrophica
MSLNLQTYTEHFTNPLYLESGRILEPYDITYETYGTMNEDKSNVVVVCHALTGSHHAAGLYEDETKPGWWDGFIGSGKAIDTDKYFVICSNVIGSCFGSTGPMSLQHPYQEPYRYKFPVVSIKDMVKAQRILFDRLDIHRVHAIVGGSMGGMQALQFAIHYPNFANKIIALATTHATQPWAIAFNKVAQESILNDPDFKQGYYDPDLLKEQGLSGMAVGRMAGHISFLSHESMREKFGRDYKLTDGLYELFGKFQVESYLEYNGYNFTKWFDPLAYLYITKAINIYDLSRGFDSLAEALKRVTSALYLVSFKNDLLFKNFEMKEIADELDKIGNKNHSYIDVKSDYGHDAFLVELNKFENHVKDALNG

SEQ ID NO: 5 - protein, 351 aa, Cyclobacterium marinum
MNLQSPHLTIEMTQEIFYCQEALSLESGESFPEFQLSFTTQGQLNANKDNVIWVLHALTGDANPHEWWSGLIGEDKFFDPSKYFIVCANFLGSCYGSTQPLSNNPNNGKPYYYDFPNITTRDIASALDKLRIHLGLEKINTVIGGSLGGQVGLEWAVSLGEKLENAIIVASNAKASPWIIGFNETQRMAIESDSTWGKTQPEAGKKGLETARAIGMLSYRHPMTFLQNQSETEEKRDDFKISSYLRYQGLKLANRFNAMSYWILSKAMDSHDIGRGRGGTPVALSNIKCKVLSIGVDTDILFTSEESRYISKHVPKGTYREISSIYGHDAFLIEYEQLQYILKSFYLENNG

SEQ ID NO: 6 - protein, 380 aa, Thermus thermophilus
MSEIALEAWGEHEALLLKPPRSPLSIPPPKPRTAVLFPRREGFYTELGGYLPEVRLRFETYGTLSRRRDNAVLVFHALTGSAHLAGTYDEETFRSLSPLEQAFGREGWWDSLVGPGRILDPALYYVVSANHLGSCYGSTGPLSLDPHTGRPYGRDFPPLTIRDLARAQARLLDHLGVEKAIVIGGSLGGMVALEFALMYPERVKKLVVLAAPARHGPWARAFNHLSRQAILQDPEYQKGNPAPKGMALARGIAMMSYRAPEGFEARWGAEPELGETYLDYQGEKFLRRFHAESYLVLSRAMDTHDVGRGRGGVEEALKRLRAIPSLFVGIDTDLLYPAWEVRQAAKAAGARYREIKSPHGHDAFLIETDQVEEILDAFLP

SEQ ID NO: 7 - protein, 434 aa, Leptospira interrogans
MPRNYKPETIALHGGQSPDPSTLSRAVPIYQTTSYVFKNTEHAAKLFGLQEFGNIYTRIMNPTTDVLEQRIAALEGGVAALATASGQAAETLALLNIVEAGQEIVASSSLYGGTYNLLHYTFPKLGIKVHFVDPSDPENFRKAVNDKTRAFYAETLGNPKLDTLNLEAIAKVAHDSEVPLIIDNTLPSPYLVNPIEHGADIVVHSLTKFLGGHGTSIGGIIVDSGKFNWGNGKFKNFTEPDPSYHGLKFWEVFGKFEPFGGVNIAYIIKAKVQGLRDMGASISPFNAWQILQGVETLPLRMRKHSENALAVAEYLTKHTKVSWVNYPGLKMDKNYSLAKKYHKKDLYGAILGFGIKGGAVEAKKFIDGLELFSLLANVGDAKSLVIHPASTTHQQLTPEEQLSAGVTPDFVRLSVGLENIEDILFDLEEALKKV

SEQ ID NO: 8 - protein, 437 aa, Corynebacterium glutamicum
MPKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTGNGSGLGGVLIDGGKFDWTVEKDGKSVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

SEQ ID NO: 9 - protein, 443 aa, Deinococcus geothermalis
MASNTLHFETLQVHAGQHPDPATGAQAVPIYATNAYVFESPEHAADLFGLRAFGNIYSRIMNPTNAVLEERIAALEGGVGALAVASGHAAQFLAITTVAQAGDNIVSTPNLYGGTVNQFRVTLRRLGIEVRFTSKDERPEEFAALIDDRTRAVYLETLGNPALNVPDFEGIAEVAHARGVAVFVDNTFGAGGYYCQPLRHGADVVLHSASKWIGGHGNGIGGLLVDGGTFDWGNGRYPLLTEPSPSYHGLSFWEAFGEGNALGLPNIAFITRARTEGLRDLGPTLAPQQAWQFLQGVETLSLRAERHAQNALALASWLSGHPDVSRVTYPGLSNHPHYDRAQTYLPRGAGAVLTFELRGGRAAGEAFIGAVRLAQHVANVGDTRTLVIHPASTTHSQLDEAAQAAAGVTPGLVRVSVGIEHIDDIREDFAQALATALVDAEGA

SEQ ID NO: 10 - protein, 421 aa, Sulfurimonas autotrophica
MDLQTLALHAGYEKDSQRTMAVPIYQTTAYEFRDVEHAANLFALKELGNIYTRLNNPTTDVFEKRFTALEGGEAAIATASGMSAIFFALANAAQAGDNIICANQLYGGSLTLSTHTLKRFGIEARFFDVHKPQQIEVLIDEKTKVIFFESLTNPSIDVADIEALTAIANKHNILTIVDNTVATPVLCRPFEFGADITVHSASKYTTGQGLAIGGILVERKGLVDKLKNNPRYSHFNEPDASYHGLVYTDTGLPPYTLRARLSLLRDLGAVVSPFNSWLFIQGIETLSLRMKEHSKNALMLAEFLESHKKVKKVNYPGLKSNANYKNAQKYFQDGLCSGLLSFEVEDFDEATKIVDATKLYSLVVNIGDSKSIITHPASTTHQQLSHEELIACGVPEGLIRISCGLESVKDLIEDLKQALEA

SEQ ID NO: 11 - protein, 434 aa, Cyclobacterium marinum
MSKNYRFETLQVHGGQEVDPTTNSRAVPIYQTSSYVFNSAEHGANLFALKEFGNIYTRIMNPTSDVFEKRMAALEGGVAAVATASGQAAQFLALNNFLSVGDNFVTSPFLYGGSYNQFKVSFKRIGIEARFAKSDKVDDLAAEINDKTKAIYVETIGNPEFNVPDFEAIAALAKKHDIPLVVDNTFGAGGYLCQPIKHGANIVTSSATKWIGGHGTSIGGIIVDGGNYNWGNGKFPQFSEPSEGYHGLNFWETFGDNNPLGLPNIAFAIRARVEGLRDFGPAISPFNSFLLLQGLETLSLRVQRTVDNALELAKWLEAHPKVKSVNYPGLTNSPYHATAKKYLTHGFGGVLSFEIEGDKETASNFINNLELISHLANVGDAKTLIIQPSATTHQQLSDEAQIAAGVTPSLLRISSGIEHIEDLKADLTAAFDKI

SEQ ID NO: 12 - protein, 421 aa, Thermus thermophilus
MRFETLQLHAGYEPEPTTLSRQVPIYPTTSYVFKSPEHAANLFALKEFGNIYSRIMNPTVDVLEKRLAALEGGKAALATASGHAAQFLALTTLAQAGDNIVSTPNLYGGTFNQFKVTLKRLGIEVRFTSREERPEEFLALTDERTRAWWVESIGNPALNIPDLEALAQAAREKGVALIVDNTFGMGGYLLRPLAWGAALVTHSLTKWVGGHGAVIAGAIVDGGSFPWEGGRYPLLTEPQPGYHGLRLTEAFGELAFIVKARVDGLRDQGQALGPFEAWVVLLGMETLSLRAERHVENTLHLAHWLLEQPQVAWVNYPGLPHHPHHDRAQKYFKGKPGAVLTFGLKGGYEAAKRFISRLKLISHLANVGDTRTLAIHPASTTHSQLSPEEQAQAGVSPEMVRLSVGLEHVEDLKAELKEALA

SEQ ID NO: 13 - DNA, 1101 bp, Synthetic construct
  source 1..1101: mol_type = other DNA
atgaacgagaccggtagcatcggcatcattgaaaccaagtacgcggagttcaaagaactgatcctgaacaacggtagcgtgctgagcccggtggttattgcgtacgagacctatggcaccctgagcagcagcaagaacaacgcgatcctgatttgccatgcgctgagcggtgatgcgcatgcggcgggttatcacagcggcagcgataagaaaccgggttggtgggacgattacattggtccgggcaagagcttcgacaccaaccagtattttatcatttgcagcaacgttatcggtggctgcaaaggtagcagcggcccgctgagcattcatccggagaccagcaccccgtatggtagccgtttcccgtttgtgagcatccaggatatggttaaggcgcaaaagctgctggttgagagcctgggtattgaaaaactgttctgcgttgcgggtggcagcatgggtggcatgcaagcgctggaatggagcatcgcgtatccgaacagcctgagcaactgcattgttatggcgagcaccgcggagcacagcgcgatgcagatcgcgtttaacgaagttggtcgtcaagcgattctgagcgacccgaactggaagaacggcctgtacgatgagaacagcccgcgtaaaggtctggcgctggcgcgtatggtgggtcacatcacctatctgagcgacgataagatgcgtgaaaaattcggtcgtaacccgccgcgtggcaacatcctgagcaccgactttgcggttggtagctacctgatttatcagggcgagagcttcgtggaccgttttgatgcgaacagctacatctatgttaccaaggcgctggaccactacagcctgggcaagggcaaagaactgaccgcggcgctgagcaacgcgacctgccgtttcctggtggttagctacagcagcgattggctgtatccgccggcgcaaagccgtgagattgtgaagagcctggaagcggcggacaaacgtgtgttctacgttgagctgcaaagcggtgaaggccacgatagctttctgctgaagaacccgaaacaaatcgagattctgaaaggttttctggaaaacccgaactaa

SEQ ID NO: 14 - DNA, 1107 bp, Synthetic construct
  source 1..1107: mol_type = other DNA
atgagcctgaacctgcaaacctataccgagcacttcaccaacccgctgtacctggagagcggtcgtatcctggaaccgtacgatattacctacgagacctatggcaccatgaacgaagacaagagcaacgtggttgtggtttgccatgcgctgaccggtagccaccatgcggcgggtctgtatgaggatgaaaccaaaccgggttggtgggacggcttcatcggtagcggcaaggcgattgacaccgataaatacttcgtgatttgcagcaacgttattggtagctgctttggtagcaccggcccgatgagcctgcaacacccgtatcaagaaccgtaccgttataagttcccggtggttagcatcaaggatatggtgaaagcgcaacgtattctgtttgaccgtctggatatccaccgtgtgcacgcgattgttggtggcagcatgggtggcatgcaggcgctgcaattcgcgatccactacccgaactttgcgaacaagatcattgcgctggcgaccacccatgcgacccagccgtgggcgatcgcgttcaacaaagtggcgcaagagagcattctgaacgacccggattttaagcagggttactatgacccggatctgctgaaagaacaaggtctgagcggtatggcggttggtcgtatggcgggtcacatcagcttcctgagccacgagagcatgcgtgaaaagtttggtcgtgattataaactgaccgacggtctgtacgagctgttcggcaagtttcaggtggagagctacctggaatataacggctacaacttcaccaagtggtttgatccgctggcgtacctgtacatcaccaaggcgatcaacatctacgatctgagccgtggtttcgacagcctggcggaggcgctgaagcgtgtgaccagcgcgctgtacctggttagctttaaaaacgacctgctgttcaagaactttgagatgaaagaaatcgcggacgaactggataagattggtaacaaaaaccacagctatatcgacgttaaaagcgattacggccacgacgcgttcctggtggagctgaacaagtttgaaaaccacgttaaagacgcgctgaacggctaa

SEQ ID NO: 15 - DNA, 1035 bp, Synthetic construct
  source 1..1035: mol_type = other DNA
atgcgtatccagcgttacattctgatgaccgcgctgatcagccaaccggacctgctgccgccgccggcgccggagcgttgcccgccgcagcaaaccgcgcgtctgttccgtgaaaccccgctgctgctggactgcggtcaggtggttcaagatgtgcgtgttgcgtaccacacctatggcaccccgagcgaccatgcgatcctggtgctgcatgcgctgaccggcaccagcgcggttcatgagtggtggccggattttctgggtgaaggcaagccgctggacccgacccgtgattatattgtttgcgcgaacgttctgggtggctgcgcgggtagcaccggtccggcggagctgccgcgtgtgaacggtgaagacccgccgctgaccctgcgtgatatggcgcgtgtgggtcgtgcgctgctggaggaactgggcgttcgtcgtgtgagcgttattggtgcgagcatgggtggcatgctggcgtatgcgtggctgctggagtgcccggacctggtggatcgtgcggttatcattggtgcgccggcgcgtcacagcccgtgggcgattggtctgaacaccgcggcgcgtaacgcgattcgtgcggcgccgggtggcgagggtctgaaggttgcgcgtcagatcgcgatgctgagctatcgtagcccggagagcttcgcgctgacccagagcggttggggcacccgtcgtccgggcaccccggacattaccacctacctggagcaccagggtgaaaaactgagcacccgtttctgcgagcgtagctatctggcgctgaccggcgcgatggaccgttttcaaccgaccgatgcggaactgcgtagcatccgtgtgccggttctggtggttggtattagcagcgatgtgctgtacccgccggcggaagtgcgtacctatgcgggtctgctgccgcgtggccagtacctggaactgcaaagcccgcacggccatgatgcgttcctgatcgatccgcagggtctgccggaagcggcggcggcgttcctgcacggtgcgtaa

SEQ ID NO: 16 - DNA, 1056 bp, Synthetic construct
  source 1..1056: mol_type = other DNA
atgaacctgcaaagcccgcacctgaccatcgagatgacccaggaaattttctactgccaagaggcgctgagcctggagagcggcgagagcttcccggaatttcaactgagctttaccacccagggccaactgaacgcgaacaaggacaacgtgatctgggttctgcacgcgctgaccggtgatgcgaacccgcacgagtggtggagcggtctgatcggcgaagacaagttctttgatccgagcaaatatttcattgtgtgcgcgaactttctgggtagctgctacggcagcacccagccgctgagcaacaacccgaacaacggtaaaccgtactattacgacttcccgaacatcaccacccgtgacattgcgagcgcgctggataagctgcgtatccacctgggcctggagaaaatcaacaccgtgattggtggcagcctgggtggccaagtgggtctggaatgggcggttagcctgggcgagaagctggaaaacgcgatcattgttgcgagcaacgcgaaagcgagcccgtggatcattggttttaacgagacccagcgtatggcgatcgaaagcgatagcacctggggcaagacccaaccggaggcgggtaagaaaggcctggaaaccgcgcgtgcgattggtatgctgagctatcgtcacccgatgaccttcctgcaaaaccaaagcgagaccgaggaaaagcgtgacgattttaaaatcagcagctatctgcgttaccagggcctgaagctggcgaaccgtttcaacgcgatgagctactggattctgagcaaagcgatggacagccacgatattggtcgtggtcgtggtggcaccccggtggcgctgagcaacatcaagtgcaaagtgctgagcatcggtgttgacaccgatattctgtttaccagcgaggaaagccgttatattagcaagcacgttccgaaaggcacctatcgtgagatcagcagcatttacggccacgacgcgttcctgatcgagtatgaacagctgcaatacattctgaagagcttctacctggaaaacaacggctaa

SEQ ID NO: 17 - DNA, 1134 bp, Synthetic construct
  source 1..1134: mol_type = other DNA
atgccgaccctggcgccgagcggtcagctggagatccaagcgattggtgacgttagcaccgaggcgggcgcgatcattaagaacgcggaaattgcgtaccaccgttggggtgagtatcgtgtggacaaagaaggccgtagcaacgtggttctgatcgaacacgcgctgaccggtgatagcaacgcggcggactggtgggcggatctgctgggtccgggcaaggcgatcaacaccgacatttactgcgttatctgcaccaacgtgatcggtggctgcaacggcagcaccggtccgggcagcatgcacccggatggtaacttctggggcaaccgttttccggcgaccagcattcgtgaccaggttaacgcggagaaacaattcctggatgcgctgggtattaccaccgttgcggcggtgctgggtggcagcatgggtggcgcgcgtaccctggagtgggcggcgatgtatccggaaatcgttggtgcggcggcggtgctggcggttagcgcgcgtgcgagcgcgtggcagatcggcattcagagcgcgcaaatcaaggcgattgaaaacgatcaccactggcacgagggtaactactatgaaagcggctgcaacccggcgaccggtctgggtgcggcgcgtcgtattgcgcacctgacctaccgtggtgagctggaaatcgacgagcgttttggcaccaaggcgcagaaaaacgaaaacccgctgggtccgtatcgtaagccggatcaacgtttcgcggttgagagctacctggactatcaggcggataaactggttcaacgttttgacgcgggtagctacgtgctgctgaccgatgcgctgaaccgtcacgacattggccgtgatcgtggtggcctgaacaaggcgctggagagcattaaagtgccggttctggtggcgggcgttgacaccgatatcctgtacctgtatcaccagcaagaacacctgagccgtaacctgggtaacctgctggcgatggcgaaaatcgttagcccggtgggtcatgatgcgttcctgaccgaaagccgtcaaatggatcgtattgtgcgtaacttctttagcctgatcagcccggacgagaacaacccgagcacctacattgaattttatatctaa

SEQ ID NO: 18 - DNA, 1167 bp, Synthetic construct
  source 1..1167: mol_type = other DNA
atgcgtatccagcgttacattctgatgagcgagatcgcgctggaagcgtggggtgagcatgaagcgctgctgctgaagccgccgcgtagcccgctgagcatcccgccgccgaagccgcgtaccgcggtgctgttcccgcgtcgtgagggtttttacaccgagctgggtggttacctgccggaagtgcgtctgcgtttcgaaacctacggcaccctgagccgtcgtcgtgataacgcggtgctggtttttcatgcgctgaccggtagcgcgcacctggcgggcacctatgatgaggaaaccttccgtagcctgagcccgctggagcaggcgtttggtcgtgaaggttggtgggatagcctggtgggtccgggtcgtatcctggacccggcgctgtactatgtggttagcgcgaaccacctgggtagctgctatggtagcaccggtccgctgagcctggacccgcacaccggtcgtccgtatggtcgtgatttcccgccgctgaccattcgtgatctggcgcgtgcgcaagcgcgtctgctggaccacctgggtgtggagaaggcgatcgttattggtggcagcctgggtggcatggtggcgctggagttcgcgctgatgtacccggaacgtgttaagaaactggtggttctggcggcgccggcgcgtcacggcccgtgggcgcgtgcgtttaaccacctgagccgtcaggcgatcctgcaagatccggaataccagaagggtaacccggcgccgaagggtatggcgctggcgcgtggtattgcgatgatgagctatcgtgcgccggaaggttttgaagcgcgttggggtgcggagccggaactgggcgagacctacctggactatcaaggtgaaaagttcctgcgtcgttttcacgcggagagctatctggtgctgagccgtgcgatggacacccatgatgtgggtcgtggtcgtggtggcgttgaggaagcgctgaaacgtctgcgtgcgatcccgagcctgtttgtgggcattgacaccgatctgctgtacccggcgtgggaagttcgtcaggcggcgaaggcggcgggtgcgcgttatcgtgagatcaaaagcccgcacggccatgatgcgttcctgatcgaaaccgatcaagttgaggaaattctggacgcgtttctgccgtaa

SEQ ID NO: 19 - DNA, 1308 bp, Synthetic construct
  source 1..1308: mol_type = other DNA
atgggtccgcgtaactataaaccggagaccattgcgctgcacggtggccagagcccggacccgagcaccctgagccgtgcggtgccgatttaccaaaccaccagctatgttttcaagaacaccgagcacgcggcgaaactgtttggtctgcaagagttcggcaacatctacacccgtattatgaacccgaccaccgatgttctggagcaacgtatcgcggcgctggaaggtggcgtggcggcgctggcgaccgcgagcggtcaggcggcggaaaccctggcgctgctgaacatcgtggaggcgggccaagaaattgttgcgagcagcagcctgtacggtggcacctataacctgctgcactatacctttccgaagctgggtatcaaagtgcacttcgttgacccgagcgatccggagaactttcgtaaggcggttaacgacaaaacccgtgcgttttacgcggaaaccctgggcaacccgaagctggataccctgaacctggaggcgattgcgaaagtggcgcacgacagcgaagttccgctgatcattgataacaccctgccgagcccgtacctggttaacccgatcgagcacggtgcggacattgtggttcacagcctgaccaagttcctgggtggtcacggcaccagcatcggtggcatcattgtggacagcggcaaatttaactggggtaacggcaagttcaaaaactttaccgaaccggacccgagctatcacggtctgaagttctgggaagtgttcggcaaatttgaaccgttcggtggcgttaacatcgcgtacatcattaaggcgaaagtgcagggtctgcgtgatatgggcgcgagcatcagcccgtttaacgcgtggcagattctgcaaggtgttgagaccctgccgctgcgtatgcgtaaacacagcgagaacgcgctggcggtggcggaatatctgaccaagcacaccaaagtgagctgggttaactacccgggtctgaagatggacaaaaactacagcctggcgaagaaatatcacaagaaagatctgtacggcgcgatcctgggtttcggcattaagggtggcgcggtggaggcgaagaaatttatcgacggtctggaactgttcagcctgctggcgaacgtgggcgatgcgaaaagcctggttattcacccggcgagcaccacccaccagcaactgaccccggaggaacaactgagcgcgggtgttaccccggacttcgtgcgtctgagcgttggcctggagaacatcgaagacattctgtttgatctggaggaagcgctgaagaaagtgtaa

SEQ ID NO: 20 - DNA, 1266 bp, Synthetic construct
  source 1..1266: mol_type = other DNA
atggacctgcaaaccctggcgctgcatgcgggttacgaaaaggatagccagcgtacgatggcggtgccgatctaccaaaccaccgcgtatgagttccgtgacgttgaacacgcggcgaacctgtttgcgctgaaggaactgggcaacatttatacccgtctgaacaacccgaccaccgatgtgttcgagaaacgttttaccgcgctggagggtggcgaagcggcgattgcgaccgcgagcggtatgagcgcgattttctttgcgctggcgaacgcggcgcaggcgggtgacaacatcatttgcgcgaaccaactgtacggtggcagcctgaccctgagcacccacaccctgaagcgtttcggtatcgaagcgcgtttctttgacgttcacaaaccgcagcaaatcgaggtgctgatcgatgaaaagaccaaggttatcttctttgaaagcctgaccaacccgagcatcgacgtggcggatattgaggcgctgaccgcgattgcgaacaaacacaacatcctgaccattgtggacaacaccgttgcgaccccggtgctgtgccgtccgtttgagtttggtgcggatatcaccgttcacagcgcgagcaagtataccaccggtcagggcctggcgatcggtggcattctggttgaacgtaaaggcctggtggacaagctgaaaaacaacccgcgttacagccacttcaacgagccggacgcgagctaccacggtctggtttataccgataccggtctgccgccgtataccctgcgtgcgcgtctgagcctgctgcgtgatctgggtgcggtggttagcccgttcaacagctggctgtttatccaaggcattgaaaccctgagcctgcgtatgaaggagcacagcaaaaacgcgctgatgctggcggagttcctggaaagccacaagaaagtgaagaaagttaactacccgggtctgaagagcaacgcgaactacaagaacgcgcagaaatattttcaagatggtctgtgcagcggcctgctgagcttcgaggtggaagactttgatgaagcgaccaagatcgttgacgcgaccaaactgtatagcctggtggttaacattggtgatagcaaaagcatcattacccatccggcgagcaccacccaccagcaactgagccacgaggaactgattgcgtgcggtgtgccggagggtctgatccgtattagctgcggcctggagagcgttaaggacctgattgaagatctgaaacaagcgctggaggcgtaa

SEQ ID NO: 21 - DNA, 1332 bp, Synthetic construct
  source 1..1332: mol_type = other DNA
atggcgagcaacaccctgcacttcgagaccctgcaagtgcacgcgggtcaacatccggacccggcgaccggcgcgcaagcggtgccgatttacgcgaccaacgcgtatgtttttgaaagcccggaacatgcggcggacctgttcggtctgcgtgcgtttggcaacatttacagccgtatcatgaacccgaccaacgcggttctggaggaacgtattgcggcgctggaaggtggcgtgggtgcgctggcggttgcgagcggtcatgcggcgcagttcctggcgattaccaccgtggcgcaagcgggtgataacatcgttagcaccccgaacctgtacggtggcaccgtgaaccaatttcgtgttaccctgcgtcgtctgggcattgaagtgcgtttcaccagcaaggacgaacgtccggaggaatttgcggcgctgatcgacgatcgtacccgtgcggtttatctggagaccctgggtaacccggcgctgaacgtgccggactttgagggtattgcggaagttgcgcatgcgcgtggcgtggcggtttttgtggataacacctttggtgcgggtggctactattgccaaccgctgcgtcacggcgcggatgtggttctgcacagcgcgagcaaatggattggtggccacggtaacggcatcggtggtctgctggttgacggtggcacctttgattggggtaacggccgttacccgctgctgaccgagccgagcccgagctatcacggtctgagcttctgggaggcgtttggtgagggtaacgcgctgggtctgccgaacattgcgttcattacccgtgcgcgtaccgaaggtctgcgtgatctgggtccgaccctggcgccgcagcaagcgtggcagtttctgcaaggtgtggagaccctgagcctgcgtgcggaacgtcatgcgcagaacgcgctggcgctggcgagctggctgagcggtcacccggatgtgagccgtgttacctatccgggcctgagcaaccacccgcactacgatcgtgcgcaaacctatctgccgcgtggtgcgggtgcggttctgacctttgagctgcgtggtggccgtgcggcgggtgaagcgtttattggtgcggtgcgtctggcgcagcatgtggcgaacgttggtgacacccgtaccctggttattcatccggcgagcaccacccacagccagctggatgaagcggcgcaagcggcggcgggtgtgaccccgggcctggttcgtgtgagcgttggtatcgagcacattgacgatatccgtgaagattttgcgcaggcgctggcgaccgcgctggttgatgcggagggtgcgtaa

SEQ ID NO: 22 - DNA, 1308 bp, Synthetic construct
  source 1..1308: mol_type = other DNA
atgggtagcaagaactaccgtttcgagaccctgcaagtgcacggtggccaagaagttgacccgaccaccaacagccgtgcggtgccgatctaccagaccagcagctatgtttttaacagcgcggagcatggtgcgaacctgttcgcgctgaaggaatttggcaacatctatacccgtattatgaacccgaccagcgacgttttcgagaaacgtatggcggcgctggaaggtggcgtggcggcggttgcgaccgcgagcggtcaggcggcgcaattcctggcgctgaacaactttctgagcgtgggcgataacttcgttaccagcccgtttctgtacggtggcagctataaccaattcaaagtgagctttaaacgtatcggtattgaggcgcgttttgcgaagagcgacaaagttgacgatctggcggcggaaatcaacgataagaccaaagcgatctacgtggagaccattggcaacccggagttcaacgttccggactttgaagcgatcgcggcgctggcgaagaaacacgacattccgctggttgtggataacaccttcggtgcgggtggctatctgtgccagccgatcaagcacggcgcgaacattgtgaccagcagcgcgaccaaatggatcggtggtcacggcaccagcattggtggcatcattgttgatggtggcaactacaactggggtaacggcaagttcccgcaatttagcgagccgagcgaaggttatcacggcctgaacttctgggagacctttggtgacaacaacccgctgggtctgccgaacattgcgttcgcgattcgtgcgcgtgtggaaggtctgcgtgattttggcccggcgatcagcccgttcaacagctttctgctgctgcaaggtctggagaccctgagcctgcgtgtgcaacgtaccgttgacaacgcgctggagctggcgaaatggctggaagcgcacccgaaggtgaaaagcgttaactatccgggtctgaccaacagcccgtaccacgcgaccgcgaagaaatatctgacccacggtttcggtggcgtgctgagctttgagattgaaggcgataaggagaccgcgagcaactttatcaacaacctggaactgattagccacctggcgaacgttggtgacgcgaaaaccctgatcattcagccgagcgcgaccacccaccagcaactgagcgatgaagcgcaaattgcggcgggtgtgaccccgagcctgctgcgtattagcagcggcatcgagcacattgaagacctgaaggcggatctgaccgcggcgttcgataaaatctaa

SEQ ID NO: 23 - DNA, 1317 bp, Synthetic construct
  source 1..1317: mol_type = other DNA
atgggtccgaagtacgacaacagcaacgcggatcagtggggcttcgagacccgtagcatccacgcgggtcagagcgttgacgcgcaaaccagcgcgcgtaacctgccgatttaccagagcaccgcgttcgtgtttgacagcgcggagcacgcgaaacaacgtttcgcgctggaagatctgggcccggtttatagccgtctgaccaacccgaccgtggaggcgctggaaaaccgtattgcgagcctggagggtggcgttcatgcggtggcgtttagcagcggtcaggcggcgaccaccaacgcgatcctgaacctggcgggtgcgggtgaccacattgttaccagcccgcgtctgtatggtggcaccgaaaccctgttcctgatcaccctgaaccgtctgggcattgatgttagctttgtggagaacccggatgatccggaaagctggcaggcggcggttcaaccgaacaccaaggcgttctttggcgagacctttgcgaacccgcaagcggacgtgctggatatcccggcggttgcggaagtggcgcaccgtaacagcgttccgctgatcattgacaacaccattgcgaccgcggcgctggtgcgtccgctggagctgggtgcggatgtggttgtggcgagcctgaccaagttctacaccggtaacggcagcggtctgggtggcgttctgatcgacggtggcaaatttgattggaccgtggaaaaggacggcaaaagcgttttcccgtattttgttaccccggatgcggcgtaccacggtctgaagtatgcggatctgggtgcgccggcgtttggtctgaaagttcgtgtgggcctgctgcgtgacaccggtagcaccctgagcgcgtttaacgcgtgggcggcggttcaaggcatcgataccctgagcctgcgtctggagcgtcacaacgaaaacgcgattaaggtggcggagttcctgaacaaccacgagaaggttgaaaaagtgaactttgcgggtctgaaggatagcccgtggtacgcgaccaaggaaaaactgggcctgaaatataccggtagcgtgctgaccttcgagatcaagggtggcaaagacgaagcgtgggcgtttattgatgcgctgaaactgcacagcaacctggcgaacatcggcgacgttcgtagcctggttgtgcatccggcgaccaccacccatagccaaagcgatgaggcgggcctggcgcgtgcgggtgtgacccaaagcaccgttcgtctgagcgtgggtatcgagaccattgacgatatcattgcggacctggaaggtggcttcgcggcgatttaa

SEQ ID NO: 24 - DNA, 1269 bp, Synthetic construct
  source 1..1269: mol_type = other DNA
atgggtcgtttcgagaccctgcaactgcatgcgggttatgagccggaaccgaccaccctgagccgtcaagttccgatctacccgaccaccagctatgtgtttaaaagcccggagcatgcggcgaacctgttcgcgctgaaggaatttggtaacatctatagccgtattatgaacccgaccgtggacgttctggagaaacgtctggcggcgctggaaggtggcaaagcggcgctggcgaccgcgagcggtcatgcggcgcagtttctggcgctgaccaccctggcgcaagcgggcgataacatcgttagcaccccgaacctgtacggtggcaccttcaaccagtttaaggttaccctgaaacgtctgggcattgaagtgcgtttcaccagccgtgaggaacgtccggaggaatttctggcgctgaccgatgagcgtacccgtgcgtggtgggtggaaagcatcggtaacccggcgctgaacattccggacctggaggcgctggcgcaagcggcgcgtgaaaaaggtgtggcgctgatcgttgataacaccttcggcatgggtggctatctgctgcgtccgctggcgtggggcgcggcgctggttacccacagcctgaccaagtgggtgggtggccacggtgcggttattgcgggtgcgattgtggatggtggcagctttccgtgggagggtggccgttacccgctgctgaccgaaccgcagccgggttatcacggcctgcgtctgaccgaggcgttcggtgaactggcgtttattgtgaaagcgcgtgttgacggcctgcgtgatcaaggtcaagcgctgggtccgtttgaggcgtgggtggttctgctgggtatggaaaccctgagcctgcgtgcggagcgtcatgtggaaaacaccctgcatctggcgcactggctgctggagcagccgcaagtggcgtgggttaactatccgggtctgccgcaccacccgcaccacgaccgtgcgcagaagtatttcaagggtaaaccgggcgcggttctgacctttggcctgaagggtggctacgaagcggcgaaacgttttatcagccgtctgaagctgattagccacctggcgaacgtgggtgacacccgtaccctggcgattcacccggcgagcaccacccacagccaactgagcccggaggaacaggcgcaagcgggtgttagcccggagatggtgcgtctgagcgttggcctggagcacgtggaagatctgaaggcggagctgaaagaagcgctggcgtaa

SEQ ID NO: 25 - DNA, 42 bp, Synthetic construct
  source 1..42: mol_type = other DNA
tttattcttgacactagtcggccaaaatgatataatacctga

SEQ ID NO: 26 - DNA, 31 bp, Synthetic construct
  source 1..31: mol_type = other DNA
caacctggaggcgggcgcaggcccgcctttt

SEQ ID NO: 27 - DNA, 14 bp, Synthetic construct
  source 1..14: mol_type = other DNA
agaggtatatatta

SEQ ID NO: 28 - DNA, 2537 bp, Synthetic construct
  source 1..2537: mol_type = other DNA
tttattcttgacactagtcggccaaaatgatataatacctgagtttaactttaagagaggtatatattaccatgggtccgcgtaactataaaccggagaccattgcgctgcacggtggccagagcccggacccgagcaccctgagccgtgcggtgccgatttaccaaaccaccagctatgttttcaagaacaccgagcacgcggcgaaactgtttggtctgcaagagttcggcaacatctacacccgtattatgaacccgaccaccgatgttctggagcaacgtatcgcggcgctggaaggtggcgtggcggcgctggcgaccgcgagcggtcaggcggcggaaaccctggcgctgctgaacatcgtggaggcgggccaagaaattgttgcgagcagcagcctgtacggtggcacctataacctgctgcactatacctttccgaagctgggtatcaaagtgcacttcgttgacccgagcgatccggagaactttcgtaaggcggttaacgacaaaacccgtgcgttttacgcggaaaccctgggcaacccgaagctggataccctgaacctggaggcgattgcgaaagtggcgcacgacagcgaagttccgctgatcattgataacaccctgccgagcccgtacctggttaacccgatcgagcacggtgcggacattgtggttcacagcctgaccaagttcctgggtggtcacggcaccagcatcggtggcatcattgtggacagcggcaaatttaactggggtaacggcaagttcaaaaactttaccgaaccggacccgagctatcacggtctgaagttctgggaagtgttcggcaaatttgaaccgttcggtggcgttaacatcgcgtacatcattaaggcgaaagtgcagggtctgcgtgatatgggcgcgagcatcagcccgtttaacgcgtggcagattctgcaaggtgttgagaccctgccgctgcgtatgcgtaaacacagcgagaacgcgctggcggtggcggaatatctgaccaagcacaccaaagtgagctgggttaactacccgggtctgaagatggacaaaaactacagcctggcgaagaaatatcacaagaaagatctgtacggcgcgatcctgggtttcggcattaagggtggcgcggtggaggcgaagaaatttatcgacggtctggaactgttcagcctgctggcgaacgtgggcgatgcgaaaagcctggttattcacccggcgagcaccacccaccagcaactgaccccggaggaacaactgagcgcgggtgttaccccggacttcgtgcgtctgagcgttggcctggagaacatcgaagacattctgtttgatctggaggaagcgctgaagaaagtgtaaggatccagaggtatatattaatgaacgagaccggtagcatcggcatcattgaaaccaagtacgcggagttcaaagaactgatcctgaacaacggtagcgtgctgagcccggtggttattgcgtacgagacctatggcaccctgagcagcagcaagaacaacgcgatcctgatttgccatgcgctgagcggtgatgcgcatgcggcgggttatcacagcggcagcgataagaaaccgggttggtgggacgattacattggtccgggcaagagcttcgacaccaaccagtattttatcatttgcagcaacgttatcggtggctgcaaaggtagcagcggcccgctgagcattcatccggagaccagcaccccgtatggtagccgtttcccgtttgtgagcatccaggatatggttaaggcgcaaaagctgctggttgagagcctgggtattgaaaaactgttctgcgttgcgggtggcagcatgggtggcatgcaagcgctggaatggagcatcgcgtatccgaacagcctgagcaactgcattgttatggcgagcaccgcggagcacagcgcgatgcagatcgcgtttaacgaagttggtcgtcaagcgattctgagcgacccgaactggaagaacggcctgtacgatgagaacagcccgcgtaaaggtctggcgctggcgcgtatggtgggtcacatcacctatctgagcgacgataagatgcgtgaaaaattcggtcgtaacccgccgcgtggcaacatcctgagcaccgactttgcggttggtagctacctgatttatcagggcgagagcttcgtggaccgttttgatgcgaacagctacatctatgttaccaaggcgctggaccactacagcctgggcaagggcaaagaactgaccgcggcgctgagcaacgcgacctgccgtttcctggtggttagctacagcagcgattggctgtatccgccggcgcaaagccgtgagattgtgaagagcctggaagcggcggacaaacgtgtgttctacgttgagctgcaaagcggtgaaggccacgatagctttctgctgaagaacccgaaacaaatcgagattctgaaaggttttctggaaaacccgaactaactcgagcaacctggaggcgggcgcaggcccgcctttt

SEQ ID NO: 29 - DNA, 2501 bp, Synthetic construct
  source 1..2501: mol_type = other DNA
tttattcttgacactagtcggccaaaatgatataatacctgagtttaactttaagagaggtatatattaccatggacctgcaaaccctggcgctgcatgcgggttacgaaaaggatagccagcgtacgatggcggtgccgatctaccaaaccaccgcgtatgagttccgtgacgttgaacacgcggcgaacctgtttgcgctgaaggaactgggcaacatttatacccgtctgaacaacccgaccaccgatgtgttcgagaaacgttttaccgcgctggagggtggcgaagcggcgattgcgaccgcgagcggtatgagcgcgattttctttgcgctggcgaacgcggcgcaggcgggtgacaacatcatttgcgcgaaccaactgtacggtggcagcctgaccctgagcacccacaccctgaagcgtttcggtatcgaagcgcgtttctttgacgttcacaaaccgcagcaaatcgaggtgctgatcgatgaaaagaccaaggttatcttctttgaaagcctgaccaacccgagcatcgacgtggcggatattgaggcgctgaccgcgattgcgaacaaacacaacatcctgaccattgtggacaacaccgttgcgaccccggtgctgtgccgtccgtttgagtttggtgcggatatcaccgttcacagcgcgagcaagtataccaccggtcagggcctggcgatcggtggcattctggttgaacgtaaaggcctggtggacaagctgaaaaacaacccgcgttacagccacttcaacgagccggacgcgagctaccacggtctggtttataccgataccggtctgccgccgtataccctgcgtgcgcgtctgagcctgctgcgtgatctgggtgcggtggttagcccgttcaacagctggctgtttatccaaggcattgaaaccctgagcctgcgtatgaaggagcacagcaaaaacgcgctgatgctggcggagttcctggaaagccacaagaaagtgaagaaagttaactacccgggtctgaagagcaacgcgaactacaagaacgcgcagaaatattttcaagatggtctgtgcagcggcctgctgagcttcgaggtggaagactttgatgaagcgaccaagatcgttgacgcgaccaaactgtatagcctggtggttaacattggtgatagcaaaagcatcattacccatccggcgagcaccacccaccagcaactgagccacgaggaactgattgcgtgcggtgtgccggagggtctgatccgtattagctgcggcctggagagcgttaaggacctgattgaagatctgaaacaagcgctggaggcgtaaggatccagaggtatatattaatgagcctgaacctgcaaacctataccgagcacttcaccaacccgctgtacctggagagcggtcgtatcctggaaccgtacgatattacctacgagacctatggcaccatgaacgaagacaagagcaacgtggttgtggtttgccatgcgctgaccggtagccaccatgcggcgggtctgtatgaggatgaaaccaaaccgggttggtgggacggcttcatcggtagcggcaaggcgattgacaccgataaatacttcgtgatttgcagcaacgttattggtagctgctttggtagcaccggcccgatgagcctgcaacacccgtatcaagaaccgtaccgttataagttcccggtggttagcatcaaggatatggtgaaagcgcaacgtattctgtttgaccgtctggatatccaccgtgtgcacgcgattgttggtggcagcatgggtggcatgcaggcgctgcaattcgcgatccactacccgaactttgcgaacaagatcattgcgctggcgaccacccatgcgacccagccgtgggcgatcgcgttcaacaaagtggcgcaagagagcattctgaacgacccggattttaagcagggttactatgacccggatctgctgaaagaacaaggtctgagcggtatggcggttggtcgtatggcgggtcacatcagcttcctgagccacgagagcatgcgtgaaaagtttggtcgtgattataaactgaccgacggtctgtacgagctgttcggcaagtttcaggtggagagctacctggaatataacggctacaacttcaccaagtggtttgatccgctggcgtacctgtacatcaccaaggcgatcaacatctacgatctgagccgtggtttcgacagcctggcggaggcgctgaagcgtgtgaccagcgcgctgtacctggttagctttaaaaacgacctgctgttcaagaactttgagatgaaagaaatcgcggacgaactggataagattggtaacaaaaaccacagctatatcgacgttaaaagcgattacggccacgacgcgttcctggtggagctgaacaagtttgaaaaccacgttaaagacgcgctgaacggctaactcgagcaacctggaggcgggcgcaggcccgcctttt

SEQ ID NO: 30 - DNA, 2495 bp, Synthetic construct
  source 1..2495: mol_type = other DNA
tttattcttgacactagtcggccaaaatgatataatacctgagtttaactttaagagaggtatatattaccatggcgagcaacaccctgcacttcgagaccctgcaagtgcacgcgggtcaacatccggacccggcgaccggcgcgcaagcggtgccgatttacgcgaccaacgcgtatgtttttgaaagcccggaacatgcggcggacctgttcggtctgcgtgcgtttggcaacatttacagccgtatcatgaacccgaccaacgcggttctggaggaacgtattgcggcgctggaaggtggcgtgggtgcgctggcggttgcgagcggtcatgcggcgcagttcctggcgattaccaccgtggcgcaagcgggtgataacatcgttagcaccccgaacctgtacggtggcaccgtgaaccaatttcgtgttaccctgcgtcgtctgggcattgaagtgcgtttcaccagcaaggacgaacgtccggaggaatttgcggcgctgatcgacgatcgtacccgtgcggtttatctggagaccctgggtaacccggcgctgaacgtgccggactttgagggtattgcggaagttgcgcatgcgcgtggcgtggcggtttttgtggataacacctttggtgcgggtggctactattgccaaccgctgcgtcacggcgcggatgtggttctgcacagcgcgagcaaatggattggtggccacggtaacggcatcggtggtctgctggttgacggtggcacctttgattggggtaacggccgttacccgctgctgaccgagccgagcccgagctatcacggtctgagcttctgggaggcgtttggtgagggtaacgcgctgggtctgccgaacattgcgttcattacccgtgcgcgtaccgaaggtctgcgtgatctgggtccgaccctggcgccgcagcaagcgtggcagtttctgcaaggtgtggagaccctgagcctgcgtgcggaacgtcatgcgcagaacgcgctggcgctggcgagctggctgagcggtcacccggatgtgagccgtgttacctatccgggcctgagcaaccacccgcactacgatcgtgcgcaaacctatctgccgcgtggtgcgggtgcggttctgacctttgagctgcgtggtggccgtgcggcgggtgaagcgtttattggtgcggtgcgtctggcgcagcatgtggcgaacgttggtgacacccgtaccctggttattcatccggcgagcaccacccacagccagctggatgaagcggcgcaagcggcggcgggtgtgaccccgggcctggttcgtgtgagcgttggtatcgagcacattgacgatatccgtgaagattttgcgcaggcgctggcgaccgcgctggttgatgcggagggtgcgtaaggatccagaggtatatattaatgcgtatccagcgttacattctgatgaccgcgctgatcagccaaccggacctgctgccgccgccggcgccggagcgttgcccgccgcagcaaaccgcgcgtctgttccgtgaaaccccgctgctgctggactgcggtcaggtggttcaagatgtgcgtgttgcgtaccacacctatggcaccccgagcgaccatgcgatcctggtgctgcatgcgctgaccggcaccagcgcggttcatgagtggtggccggattttctgggtgaaggcaagccgctggacccgacccgtgattatattgtttgcgcgaacgttctgggtggctgcgcgggtagcaccggtccggcggagctgccgcgtgtgaacggtgaagacccgccgctgaccctgcgtgatatggcgcgtgtgggtcgtgcgctgctggaggaactgggcgttcgtcgtgtgagcgttattggtgcgagcatgggtggcatgctggcgtatgcgtggctgctggagtgcccggacctggtggatcgtgcggttatcattggtgcgccggcgcgtcacagcccgtgggcgattggtctgaacaccgcggcgcgtaacgcgattcgtgcggcgccgggtggcgagggtctgaaggttgcgcgtcagatcgcgatgctgagctatcgtagcccggagagcttcgcgctgacccagagcggttggggcacccgtcgtccgggcaccccggacattaccacctacctggagcaccagggtgaaaaactgagcacccgtttctgcgagcgtagctatctggcgctgaccggcgcgatggaccgttttcaaccgaccgatgcggaactgcgtagcatccgtgtgccggttctggtggttggtattagcagcgatgtgctgtacccgccggcggaagtgcgtacctatgcgggtctgctgccgcgtggccagtacctggaactgcaaagcccgcacggccatgatgcgttcctgatcgatccgcagggtctgccggaagcggcggcggcgttcctgcacggtgcgtaactcgagcaacctggaggcgggcgcaggcccgcctttt

SEQ ID NO: 31 - DNA, 2492 bp, Synthetic construct
  source 1..2492: mol_type = other DNA
tttattcttgacactagtcggccaaaatgatataatacctgagtttaactttaagagaggtatatattaccatgggtagcaagaactaccgtttcgagaccctgcaagtgcacggtggccaagaagttgacccgaccaccaacagccgtgcggtgccgatctaccagaccagcagctatgtttttaacagcgcggagcatggtgcgaacctgttcgcgctgaaggaatttggcaacatctatacccgtattatgaacccgaccagcgacgttttcgagaaacgtatggcggcgctggaaggtggcgtggcggcggttgcgaccgcgagcggtcaggcggcgcaattcctggcgctgaacaactttctgagcgtgggcgataacttcgttaccagcccgtttctgtacggtggcagctataaccaattcaaagtgagctttaaacgtatcggtattgaggcgcgttttgcgaagagcgacaaagttgacgatctggcggcggaaatcaacgataagaccaaagcgatctacgtggagaccattggcaacccggagttcaacgttccggactttgaagcgatcgcggcgctggcgaagaaacacgacattccgctggttgtggataacaccttcggtgcgggtggctatctgtgccagccgatcaagcacggcgcgaacattgtgaccagcagcgcgaccaaatggatcggtggtcacggcaccagcattggtggcatcattgttgatggtggcaactacaactggggtaacggcaagttcccgcaatttagcgagccgagcgaaggttatcacggcctgaacttctgggagacctttggtgacaacaacccgctgggtctgccgaacattgcgttcgcgattcgtgcgcgtgtggaaggtctgcgtgattttggcccggcgatcagcccgttcaacagctttctgctgctgcaaggtctggagaccctgagcctgcgtgtgcaacgtaccgttgacaacgcgctggagctggcgaaatggctggaagcgcacccgaaggtgaaaagcgttaactatccgggtctgaccaacagcccgtaccacgcgaccgcgaagaaatatctgacccacggtttcggtggcgtgctgagctttgagattgaaggcgataaggagaccgcgagcaactttatcaacaacctggaactgattagccacctggcgaacgttggtgacgcgaaaaccctgatcattcagccgagcgcgaccacccaccagcaactgagcgatgaagcgcaaattgcggcgggtgtgaccccgagcctgctgcgtattagcagcggcatcgagcacattgaagacctgaaggcggatctgaccgcggcgttcgataaaatctaaggatccagaggtatatattaatgaacctgcaaagcccgcacctgaccatcgagatgacccaggaaattttctactgccaagaggcgctgagcctggagagcggcgagagcttcccggaatttcaactgagctttaccacccagggccaactgaacgcgaacaaggacaacgtgatctgggttctgcacgcgctgaccggtgatgcgaacccgcacgagtggtggagcggtctgatcggcgaagacaagttctttgatccgagcaaatatttcattgtgtgcgcgaactttctgggtagctgctacggcagcacccagccgctgagcaacaacccgaacaacggtaaaccgtactattacgacttcccgaacatcaccacccgtgacattgcgagcgcgctggataagctgcgtatccacctgggcctggagaaaatcaacaccgtgattggtggcagcctgggtggccaagtgggtctggaatgggcggttagcctgggcgagaagctggaaaacgcgatcattgttgcgagcaacgcgaaagcgagcccgtggatcattggttttaacgagacccagcgtatggcgatcgaaagcgatagcacctggggcaagacccaaccggaggcgggtaagaaaggcctggaaaccgcgcgtgcgattggtatgctgagctatcgtcacccgatgaccttcctgcaaaaccaaagcgagaccgaggaaaagcgtgacgattttaaaatcagcagctatctgcgttaccagggcctgaagctggcgaaccgtttcaacgcgatgagctactggattctgagcaaagcgatggacagccacgatattggtcgtggtcgtggtggcaccccggtggcgctgagcaacatcaagtgcaaagtgctgagcatcggtgttgacaccgatattctgtttaccagcgaggaaagccgttatattagcaagcacgttccgaaaggcacctatcgtgagatcagcagcatttacggccacgacgcgttcctgatcgagtatgaacagctgcaatacattctgaagagcttctacctggaaaacaacggctaactcgagcaacctggaggcgggcgcaggcccgcctttt

SEQ ID NO: 32 - DNA, 2579 bp, Synthetic construct
  source 1..2579: mol_type = other DNA
tttattcttgacactagtcggccaaaatgatataatacctgagtttaactttaagagaggtatatattaccatgggtccgaagtacgacaacagcaacgcggatcagtggggcttcgagacccgtagcatccacgcgggtcagagcgttgacgcgcaaaccagcgcgcgtaacctgccgatttaccagagcaccgcgttcgtgtttgacagcgcggagcacgcgaaacaacgtttcgcgctggaagatctgggcccggtttatagccgtctgaccaacccgaccgtggaggcgctggaaaaccgtattgcgagcctggagggtggcgttcatgcggtggcgtttagcagcggtcaggcggcgaccaccaacgcgatcctgaacctggcgggtgcgggtgaccacattgttaccagcccgcgtctgtatggtggcaccgaaaccctgttcctgatcaccctgaaccgtctgggcattgatgttagctttgtggagaacccggatgatccggaaagctggcaggcggcggttcaaccgaacaccaaggcgttctttggcgagacctttgcgaacccgcaagcggacgtgctggatatcccggcggttgcggaagtggcgcaccgtaacagcgttccgctgatcattgacaacaccattgcgaccgcggcgctggtgcgtccgctggagctgggtgcggatgtggttgtggcgagcctgaccaagttctacaccggtaacggcagcggtctgggtggcgttctgatcgacggtggcaaatttgattggaccgtggaaaaggacggcaaaagcgttttcccgtattttgttaccccggatgcggcgtaccacggtctgaagtatgcggatctgggtgcgccggcgtttggtctgaaagttcgtgtgggcctgctgcgtgacaccggtagcaccctgagcgcgtttaacgcgtgggcggcggttcaaggcatcgataccctgagcctgcgtctggagcgtcacaacgaaaacgcgattaaggtggcggagttcctgaacaaccacgagaaggttgaaaaagtgaactttgcgggtctgaaggatagcccgtggtacgcgaccaaggaaaaactgggcctgaaatataccggtagcgtgctgaccttcgagatcaagggtggcaaagacgaagcgtgggcgtttattgatgcgctgaaactgcacagcaacctggcgaacatcggcgacgttcgtagcctggttgtgcatccggcgaccaccacccatagccaaagcgatgaggcgggcctggcgcgtgcgggtgtgacccaaagcaccgttcgtctgagcgtgggtatcgagaccattgacgatatcattgcggacctggaaggtggcttcgcggcgatttaaggatccagaggtatatattaatgccgaccctggcgccgagcggtcagctggagatccaagcgattggtgacgttagcaccgaggcgggcgcgatcattaagaacgcggaaattgcgtaccaccgttggggtgagtatcgtgtggacaaagaaggccgtagcaacgtggttctgatcgaacacgcgctgaccggtgatagcaacgcggcggactggtgggcggatctgctgggtccgggcaaggcgatcaacaccgacatttactgcgttatctgcaccaacgtgatcggtggctgcaacggcagcaccggtccgggcagcatgcacccggatggtaacttctggggcaaccgttttccggcgaccagcattcgtgaccaggttaacgcggagaaacaattcctggatgcgctgggtattaccaccgttgcggcggtgctgggtggcagcatgggtggcgcgcgtaccctggagtgggcggcgatgtatccggaaatcgttggtgcggcggcggtgctggcggttagcgcgcgtgcgagcgcgtggcagatcggcattcagagcgcgcaaatcaaggcgattgaaaacgatcaccactggcacgagggtaactactatgaaagcggctgcaacccggcgaccggtctgggtgcggcgcgtcgtattgcgcacctgacctaccgtggtgagctggaaatcgacgagcgttttggcaccaaggcgcagaaaaacgaaaacccgctgggtccgtatcgtaagccggatcaacgtttcgcggttgagagctacctggactatcaggcggataaactggttcaacgttttgacgcgggtagctacgtgctgctgaccgatgcgctgaaccgtcacgacattggccgtgatcgtggtggcctgaacaaggcgctggagagcattaaagtgccggttctggtggcgggcgttgacaccgatatcctgtacctgtatcaccagcaagaacacctgagccgtaacctgggtaacctgctggcgatggcgaaaatcgttagcccggtgggtcatgatgcgttcctgaccgaaagccgtcaaatggatcgtattgtgcgtaacttctttagcctgatcagcccggacgagaacaacccgagcacctacattgaattttatatctaactcgagcaacctggaggcgggcgcaggcccgcctttt

SEQ ID NO: 33 - DNA, 2564 bp, Synthetic construct
  source 1..2564: mol_type = other DNA
tttattcttgacactagtcggccaaaatgatataatacctgagtttaactttaagagaggtatatattaccatgggtcgtttcgagaccctgcaactgcatgcgggttatgagccggaaccgaccaccctgagccgtcaagttccgatctacccgaccaccagctatgtgtttaaaagcccggagcatgcggcgaacctgttcgcgctgaaggaatttggtaacatctatagccgtattatgaacccgaccgtggacgttctggagaaacgtctggcggcgctggaaggtggcaaagcggcgctggcgaccgcgagcggtcatgcggcgcagtttctggcgctgaccaccctggcgcaagcgggcgataacatcgttagcaccccgaacctgtacggtggcaccttcaaccagtttaaggttaccctgaaacgtctgggcattgaagtgcgtttcaccagccgtgaggaacgtccggaggaatttctggcgctgaccgatgagcgtacccgtgcgtggtgggtggaaagcatcggtaacccggcgctgaacattccggacctggaggcgctggcgcaagcggcgcgtgaaaaaggtgtggcgctgatcgttgataacaccttcggcatgggtggctatctgctgcgtccgctggcgtggggcgcggcgctggttacccacagcctgaccaagtgggtgggtggccacggtgcggttattgcgggtgcgattgtggatggtggcagctttccgtgggagggtggccgttacccgctgctgaccgaaccgcagccgggttatcacggcctgcgtctgaccgaggcgttcggtgaactggcgtttattgtgaaagcgcgtgttgacggcctgcgtgatcaaggtcaagcgctgggtccgtttgaggcgtgggtggttctgctgggtatggaaaccctgagcctgcgtgcggagcgtcatgtggaaaacaccctgcatctggcgcactggctgctggagcagccgcaagtggcgtgggttaactatccgggtctgccgcaccacccgcaccacgaccgtgcgcagaagtatttcaagggtaaaccgggcgcggttctgacctttggcctgaagggtggctacgaagcggcgaaacgttttatcagccgtctgaagctgattagccacctggcgaacgtgggtgacacccgtaccctggcgattcacccggcgagcaccacccacagccaactgagcccggaggaacaggcgcaagcgggtgttagcccggagatggtgcgtctgagcgttggcctggagcacgtggaagatctgaaggcggagctgaaagaagcgctggcgtaaggatccagaggtatatattaatgcgtatccagcgttacattctgatgagcgagatcgcgctggaagcgtggggtgagcatgaagcgctgctgctgaagccgccgcgtagcccgctgagcatcccgccgccgaagccgcgtaccgcggtgctgttcccgcgtcgtgagggtttttacaccgagctgggtggttacctgccggaagtgcgtctgcgtttcgaaacctacggcaccctgagccgtcgtcgtgataacgcggtgctggtttttcatgcgctgaccggtagcgcgcacctggcgggcacctatgatgaggaaaccttccgtagcctgagcccgctggagcaggcgtttggtcgtgaaggttggtgggatagcctggtgggtccgggtcgtatcctggacccggcgctgtactatgtggttagcgcgaaccacctgggtagctgctatggtagcaccggtccgctgagcctggacccgcacaccggtcgtccgtatggtcgtgatttcccgccgctgaccattcgtgatctggcgcgtgcgcaagcgcgtctgctggaccacctgggtgtggagaaggcgatcgttattggtggcagcctgggtggcatggtggcgctggagttcgcgctgatgtacccggaacgtgttaagaaactggtggttctggcggcgccggcgcgtcacggcccgtgggcgcgtgcgtttaaccacctgagccgtcaggcgatcctgcaagatccggaataccagaagggtaacccggcgccgaagggtatggcgctggcgcgtggtattgcgatgatgagctatcgtgcgccggaaggttttgaagcgcgttggggtgcggagccggaactgggcgagacctacctggactatcaaggtgaaaagttcctgcgtcgttttcacgcggagagctatctggtgctgagccgtgcgatggacacccatgatgtgggtcgtggtcgtggtggcgttgaggaagcgctgaaacgtctgcgtgcgatcccgagcctgtttgtgggcattgacaccgatctgctgtacccggcgtgggaagttcgtcaggcggcgaaggcggcgggtgcgcgttatcgtgagatcaaaagcccgcacggccatgatgcgttcctgatcgaaaccgatcaagttgaggaaattctggacgcgtttctgccgtaactcgagcaacctggaggcgggcgcaggcccgcctttt

SEQ ID NO: 34 - DNA, 29 bp, Synthetic construct
  source 1..29: mol_type = other DNA
gtttaactttaagagaggtatatattacc

SEQ ID NO: 35 - DNA, 20 bp, Synthetic construct
  source 1..20: mol_type = other DNA
ggatccagaggtatatatta

SEQ ID NO: 36 - DNA, 70 bp, Synthetic construct
  source 1..70: mol_type = other DNA
cgagctacccgccgtcaatttcttgcgtgaagaaaacgtctttgtgatgacatatgaatatcctccttag

SEQ ID NO: 37 - DNA, 70 bp, Synthetic construct
  source 1..70: mol_type = other DNA
tgtgccgtagatcgtatggcgtgatctggtagacgtaatagttgagccagtgtaggctggagctgcttcg

SEQ ID NO: 38 - DNA, 70 bp, Synthetic construct
  source 1..70: mol_type = other DNA
acaggccaccatcgcagtgcgtagcgggttaaatgacgacgaacagtatgcatatgaatatcctccttag

SEQ ID NO: 39 - DNA, 70 bp, Synthetic construct
  source 1..70: mol_type = other DNA
cggaagccattttccaggtcggcaattaaatcttcgccatcttcaatacctgtaggctggagctgcttcg

SEQ ID NO: 40 - DNA, 70 bp, Synthetic construct
  source 1..70: mol_type = other DNA
ggagcggcgaatatatcagcccatacgctgagcacggcaagaagagtgaatgtaggctggagctgcttcg

SEQ ID NO: 41 - DNA, 70 bp, Synthetic construct
  source 1..70: mol_type = other DNA
cacgtctccgggttaatccccatctcacgcatgatctcttttgccgcttccatatgaatatcctccttag

SEQ ID NO: 42 - DNA, 20 bp, Synthetic construct
  source 1..20: mol_type = other DNA
gttatgccgattcgtgtgcc

SEQ ID NO: 43 - DNA, 20 bp, Synthetic construct
  source 1..20: mol_type = other DNA
aggtaaggtgctgaatcgct

SEQ ID NO: 44 - DNA, 20 bp, Synthetic construct
  source 1..20: mol_type = other DNA
acatttcaccgacaaagccc

SEQ ID NO: 45 - DNA, 20 bp, Synthetic construct
  source 1..20: mol_type = other DNA
tttaccccttgtttgcagcc

SEQ ID NO: 46 - DNA, 20 bp, Synthetic construct
  source 1..20: mol_type = other DNA
actgtgtggtctggtctcaa

SEQ ID NO: 47 - DNA, 20 bp, Synthetic construct
  source 1..20: mol_type = other DNA
ctgataagcgtagcgcatca

SEQ ID NO: 48 - DNA, 31 bp, Synthetic construct
  source 1..31: mol_type = other DNA
gcgccatggatgagtggactcaaacaagaac

SEQ ID NO: 49 - DNA, 31 bp, Synthetic construct
  source 1..31: mol_type = other DNA
gcgctcgagttatgtggttatgccattttcc

SEQ ID NO: 50 - protein, 376 aa, Corynebacterium glutamicum
PTLAPSGQLEIQAIGDVSTEAGAIIKNAEIAYHRWGEYRVDKEGRSNVVLIEHALTGDSNAADWWADLLGPGKAINTDIYCVICTNVIGGCNGSTGPGSMHPDGNFWGNRFPATSIRDQVNAEKQFLDALGITTVAAVLGGSMGGARTLEWAAMYPEIVGAAAVLAVSARASAWQIGIQSAQIKAIENDHHWHEGNYYESGCNPATGLGAARRIAHLTYRGELEIDERFGTKAQKNENPLGPYRKPDQRFAVESYLDYQADKLVQRFDAGSYVLLTDALNRHDIGRDRGGLNKALESIKVPVLVAGVDTDILYPYHQQEHLSRNLGNLLAMAKIVSPVGHDAFLTESRQMDRIVRNFFSLISPDENNPSTYIEFYI

(19) United States
(12) Patent Application Publication
(10) Pub. No.: US 2026/0226403 A1
(43) Pub. Date: Aug. 6, 2026

(54) BACTERIA AND A METHOD OF USING SAME FOR AMINO ACIDS BIOSYNTHESIS

(71) Applicants: MIGAL GALILEE RESEARCH INSTITUTE LTD., Kiryat Shmona (IL); RAMOT AT TEL-AVIV UNIVERSITY LTD., Tel Aviv (IL)

(72) Inventors: Maayan GAL, Tel Aviv (IL); Rachel AMIR, Kibbutz Dan (IL); Yael HACHAM, Tiberias (IL); Nadia GRUZDEV, Kidmat Zvi (IL); Hadar HAVIV, Kidmat Zvi (IL); Itamar YADID, Ayelet HaShachar (IL); Itai BLOCH, Ramot-Naftali (IL)

(21) Appl. No.: 19/141,711

(22) PCT Filed: Dec. 21, 2023
(86) PCT No.: PCT/IL2023/051295

§ 371 Date: Jun. 20, 2025

Related U.S. Application Data
(60) Provisional application No. 63/434,913, filed on Dec. 22, 2022.

(51) Int. Cl. C12N 1/20; C07K 14/245; C12N 9/10; C12N 15/52; C12N 15/70; C12P 13/12; C12R 1/19

(52) U.S. Cl. CPC C12N 1/20; C07K 14/245; C12N 9/1029; C12N 9/1085; C12N 15/52; C12N 15/70; C12P 13/12; C12Y 203/01031; C12Y 203/01046; C12Y 205/01048; C12Y 205/01049; C12N 2800/101; C12R 2001/19

(57) ABSTRACT

The present invention provides is directed to a method of producing an amino acid, including: (a) providing a bacterial cell including an exogenous nucleic acid molecule or a plurality thereof encoding metX and metY; and culturing the bacterial cell from step (a) such that metX and metY encoded by an exogenous nucleic acid molecule are expressed, bacterial cell including: (i) an exogenous nucleic acid molecule or a plurality thereof, encoding homoserine O-acetyltransferase (metX) and O-acetylhomoserine sulfhydrylase (metY). Further provided are a composition comprising the bacterial cell, and a method for producing amino acids.

### CROSS-REFERENCE TO RELATED-APPLICATIONS

This application claims the benefit of priority of U.S. Provisional Patent Application No. 63/434,913, titled “BACTERIA AND A METHOD OF USING SAME FOR AMINO ACIDS BIOSYNTHESIS”, filed on 22 Dec. 2022, the contents of which are incorporated herein by reference in their entirety.

### REFERENCE TO AN ELECTRONIC SEQUENCE LISTING

The contents of the electronic sequence listing (MIG-RMT-P-026-PCT ST26.xml; size: 79,562 bytes; and date of creation: Nov. 23, 2023) is herein incorporated by reference in its entirety.

### FIELD OF INVENTION

The present invention is in the field of biosynthesis of amino acids in microorganisms.

### BACKGROUND OF THE INVENTION

Methionine is a sulfur-containing amino acid synthesized by plants, fungi and bacteria but not by vertebrates and thus considered an essential amino acid. Methionine plays a major role in mRNA translation initiation and is the precursor for the central metabolite S-adenosyl-methionine (SAM) that is part of the methionine biosynthesis regulation. Although methionine is a relatively nonabundant amino acid in proteins, it is a hydrophobic building block that stabilizes the overall structure of proteins. Besides being the first amino acid in protein synthesis, it is a key factor for the synthesis of a wide range of metabolites. Thus, L-Methionine is an important molecule for food and feed purposes as well as for the pharmaceutical industry. Despite the growing need for L-methionine, the main approach to obtain methionine is by chemical synthesis, resulting in a racemic mixture of the D, L forms of methionine.

The metabolic cost of methionine biosynthesis in microorganisms that utilizes the trans-sulfurylation pathway is high and its synthesis and consumption are under strict regulation.

Therefore, production of L-methionine beyond physiological levels is challenging. There is an unmet need for developing an efficient bio-fermentation process for L-methionine synthesis.

### SUMMARY OF THE INVENTION

According to one aspect, there is provided a bacterial cell comprising: (i) an exogenous nucleic acid molecule or a plurality thereof, encoding homoserine O-acetyltransferase (metX) and O-acetylhomoserine sulfhydrylase (metY), and any one of: (ii) inactive homoserine O-succinyltransferase (metA) and cystathionine gamma-synthase (metB) genes; (iii) an inactive metJ methionine repressor gene, or both (ii) and (iii).

According to another aspect, there is provided a composition comprising the bacterial cell disclosed herein, and a biologically accepted carrier.

According to another aspect, there is provided a method of producing an amino acid, the method comprising: (a) providing a bacterial cell comprising: (i) an exogenous nucleic acid molecule or a plurality thereof encoding metX and metY, and (ii) inactive metA and metB genes; and, (b) culturing the bacterial cell from step (a) such that metX and metY encoded by the exogenous nucleic acid molecule are expressed, thereby, producing the amino acid.

In some embodiments, any one of the inactive genes is knocked out, knocked down, mutated, or chemically inhibited.

In some embodiments, the bacterial cell further comprising one or more inhibitory nucleic acid sequences being complementary to any one of: metA, metB, and metJ genes.

In some embodiments, the inactive gene being mutated comprises a nucleic acid sequence comprising a premature stop codon compared to a wildtype form of the gene.

In some embodiments, the encoded metX comprises the amino acid sequence set forth in any one of SEQ ID Nos: 1-6, and 50.

In some embodiments, the encoded metY comprises the amino acid sequence set forth in any one of SEQ ID Nos: 7-12.

In some embodiments, the exogenous nucleic acid molecule or a plurality thereof, comprises a nucleic acid sequence being codon optimized for expression in the bacterial cell.

In some embodiments, the bacterial cell comprises an Escherichia coli cell.

In some embodiments, the exogenous nucleic acid molecule encoding metX comprises the nucleotide sequence set forth in any one of SEQ ID Nos: 13-18.

In some embodiments, the exogenous nucleic acid molecule encoding metY comprises the nucleotide sequence set forth in any one of SEQ ID Nos: 19-24.

In some embodiments, the encoded metY comprises the amino acid sequence set forth in any one of SEQ ID Nos: 7-12.

In some embodiments, the encoded metY comprises the amino acid sequence set forth in any one of SEQ ID Nos: 9 and 11.

In some embodiments, the exogenous nucleic acid molecule or plurality thereof encoding metX and metY are operably linked.

In some embodiments, the exogenous nucleic acid molecule or plurality thereof, is: (i) integrated into the genome of the bacterial cell; or is comprised within a plasmid or an expression vector.

In some embodiments, the exogenous nucleic acid molecule or a plurality thereof comprises the nucleic acid sequence set forth in any one of SEQ ID Nos: 25-30.

In some embodiments, the encoded metX, comprises the amino acid sequence set forth in any one of SEQ ID Nos: 3 and 5, and the encoded metY comprises the amino acid sequence set forth in any one of SEQ ID Nos: 9 and 11.

In some embodiments, the exogenous nucleic acid molecule encoding the metX comprises the nucleotide sequence set forth in any one of SEQ ID Nos: 15 and 16, and the exogenous nucleic acid molecule encoding the metY comprises the nucleotide sequence set forth in any one of SEQ ID Nos: 21 and 22.

In some embodiments, the bacterial cell further comprises an inactive metJ.

In some embodiments, the bacterial cell is further characterized by expression of the yjeH gene.

In some embodiments, the bacterial cell comprises or further comprises an L-methionine and branched chain amino acid (BCAA) exporter protein.

In some embodiments, the amino acid comprises an essential amino acid.

In some embodiments, the essential amino acid is selected from the group consisting of: methionine, alanine, valine, leucine, threonine, isoleucine, glutamate, and any combination thereof.

In some embodiments, culturing comprises supplementing the bacterial cell with a culture medium comprising an effective amount of a sulfur compound.

In some embodiments, the produced levels of the L-methionine being produced by the bacterial cell are at least 5-fold greater than levels of L-methionine being produced by a control bacterial cell.

In some embodiments, produced levels comprises intracellular levels, extracellular levels, or both.

In some embodiments, the produced levels of the amino acid comprises intracellular levels, extracellular levels, or both, of the amino acid.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a schematic illustration of direct- and trans-sulfurylation of methionine biosynthesis in bacteria. The first step involves the activation of homoserine through an acylation step. Two enzymes can process the activated homo-serine. The enzyme L-homoserine O-succinyl transferase (HST; EC 2.3.1.46) converts homoserine and succinyl-CoA to O-succinyl-L-homoserine (OSH). The enzyme L-homoserine O-acetyltransferases (HAT; EC 2.3.1.31) converts homoserine and acetyl-CoA to generate O-acetyl-L-homoserine (OAH). These enzymes are encoded by the MetA and MetX genes. In the trans-sulfurylation pathway the sulfur donor cysteine together with OSH/OAH are converted to cystathionine. This step is catalyzed by the cystathionine gamma synthase (CgS; EC 2.5.1.48) encoded by the MetB gene. Cystathionine is further converted to homocysteine by the enzyme cystathionine beta lyase (CbL; EC 4.4.1.13) encoded by MetC. In the direct sulfurylation path, OAH is converted to homocysteine within a single step. This reaction is catalyzed by the enzyme O-acetylhomoserine sulfhydrylase (OAHS; EC 2.5.1.49) encoded by MetY. Bue boxes-common metabolites; Green boxes-step in trans-sulfurylation pathway; Brown—Direct sulfurylation steps. MetJ and mcbr are master negative regulators in E. coli and C. glutamicum, respectively.

FIG. 2 includes curve graphs demonstrating the growth rate of auxotroph E. coli AMetAB with variable methionine concentrations (indicated in the legend in μM).

FIGS. 3A-3D include images and graphs demonstrating the complementation of engineered E. coli with MetX/Y. (3A) Synthetic metYX operon on a low copy plasmid. A synthetic operon consisting of the metY and metX genes was constructed by adding a constitutive promoter, RBSand a synthetic terminator for each gene. Restriction sites were included to facilitate the rearrangement and analysis of mutant genes. (3B) Growth curves of the complemented ΔMetAB strains on methionine-depleted minimal media. (3C) Growth of the complemented ΔMetAB strains on a methionine-depleted minimal media agar plate. (3D) Intracellular (left) methionine accumulated by WT E. coli, ΔmetAB-DG and ΔmetAB-CM, reported as μg/ml, and extracellular (right) methionine accumulated in the growth media by WT E. coli, ΔmetAB-DG and ΔmetAB-CM, reported as μg/ml. The results are presented as means±SD of three to four replicates for each sample. Significance between WT and the different bacterial strain was calculated according to the Student's t-test (P<0.05) and is identified by an asterisk. The numbers on top of the bars indicate the fold increase relative to the WT in each panel.

FIGS. 4A-4C include bar graphs showing production of methionine by ΔmetABJ overexpressing YjeH and complemented by metY/X from CM or DG. Comparison of: (4A) intracellular; (4B) extracellular; and (4C) total methionine levels that were quantified by GC-MS. Peak areas were normalized to Norleucine internal control, and total methionine levels were calculated according to the standard calibration curves. The results are presented as means±SD of three or four replicates for each sample. Significance between bacterial strains was calculated according to the Turkey-Kramer HSD test (p<0.05) and is identified by different small letters. Significance between WT and the different bacterial strains was calculated according to the Student's t-test (P<0.05) and is identified by an asterisk. The numbers on top of the bars in each panel indicate the fold-increase relative to the WT in each panel.

FIGS. 5A-5C include a non-limiting scheme and curves showing growth of methionine-auxotroph E. coli in spent medium of each strain. (5A) Non-limiting experimental design used to evaluate methionine level in the medium following the growth of each strain. (5B) Growth curves in medium from DG strains. (5C) Growth curves in medium from CM strains. All curves show the growth of the auxotroph E. coli ΔmetAB in fresh MOPS minimal medium supplemented with spent medium filtered following the growth of the indicated strains.

FIGS. 6A-6B include growth curves of methionine-auxotroph E. coli in spent medium of modified strains. (6A) Growth curves of a methionine auxotroph E. coli strain (DmetAB) supplemented with spent medium retrieved from cultivation of an E. coli strain deleted of metJ and expressing metXmetY from a plasmid (metJ+DG), and of an E. coli strain deleted of metABJ and expressing metXmetY from a plasmid (metABJ+DG). (6B) Growth curves of a methionine auxotroph E. coli strain (DmetAB) supplemented with the spent medium following the cultivation of an E. coli strain deleted of metJ and expressing yjeH and metXmetY from a plasmid (J DG yjeH), of an E. coli strain deleted of metABJ and expressing yjeHl and MetYX from a plasmid (ABJ DG yjeH), or supplemented with 0, 2, and 5 mg/ml free methionine (met0, met2 and met5, respectively). metJ-knockout of the metJ gene; metAB-knockout of metA and metB genes; metABJ-knockout of metA, metB and metJ genes; DG—metX and metY genes from D. geothermalis expressed from a plasmid; and yjeH—a gene encoding for methionine transporter expressed from a plasmid.

### DETAILED DESCRIPTION OF THE INVENTION

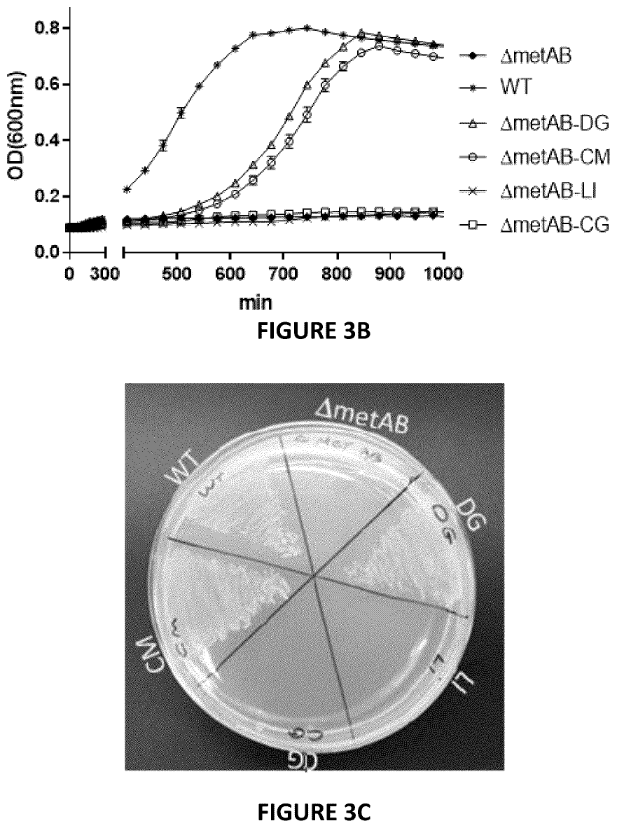

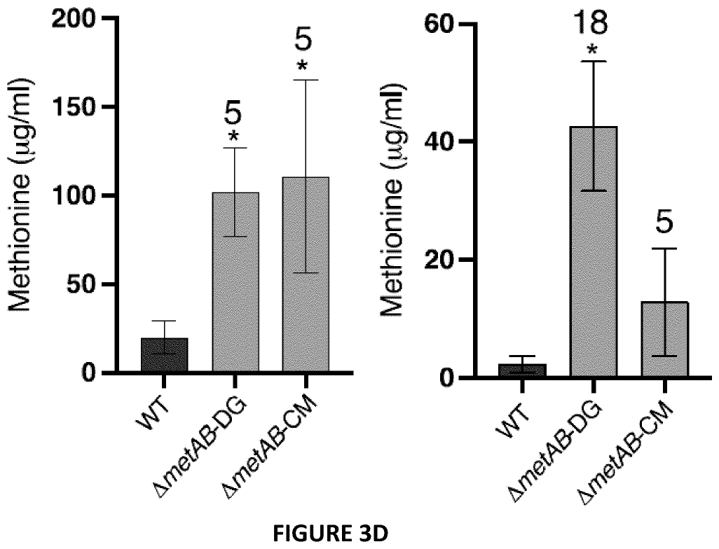

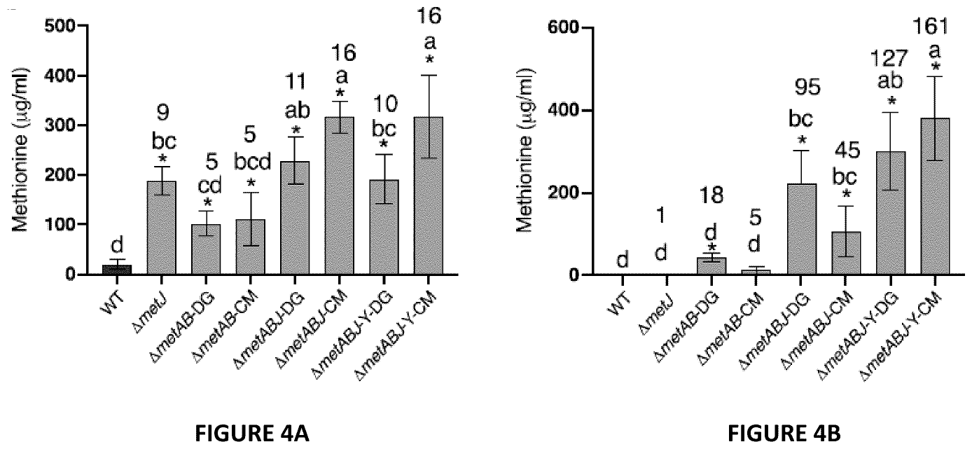

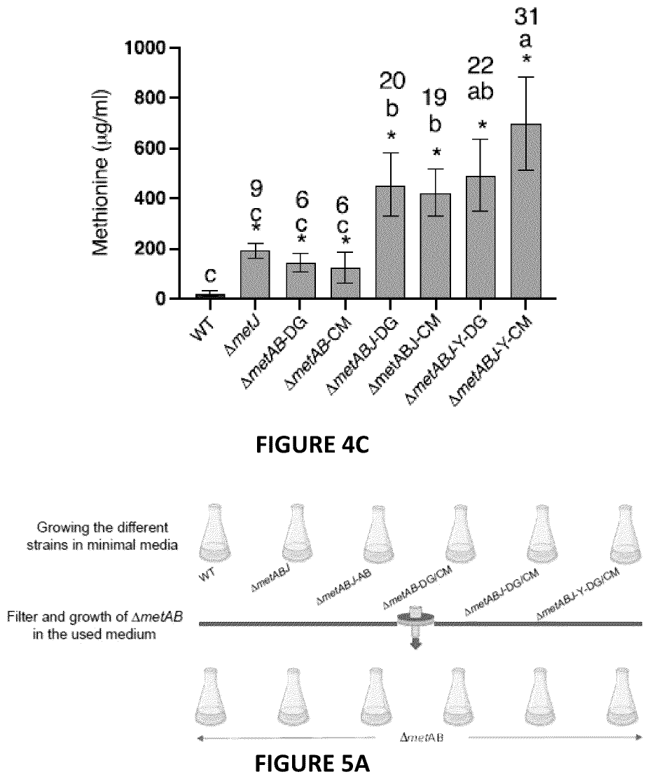

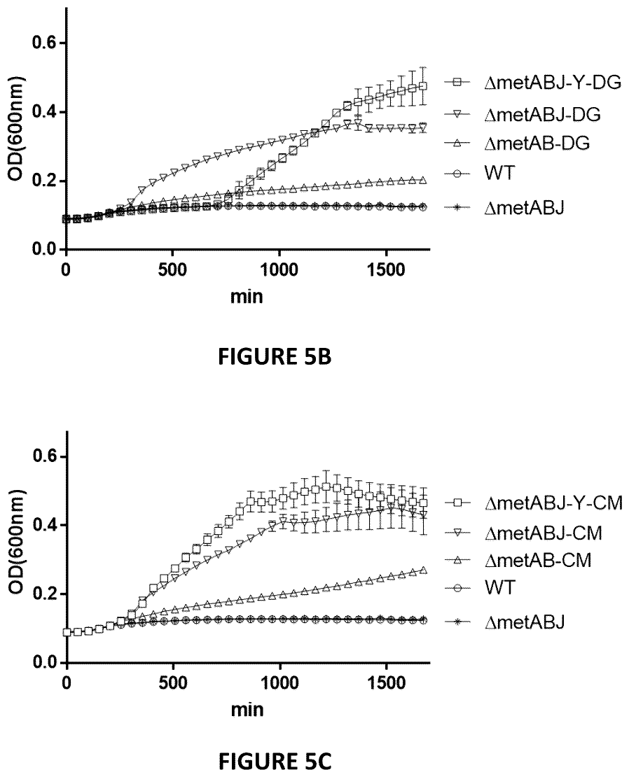

The present invention, in some embodiments, is partially based on the finding that inactivating metA and metB genes of E. coli, and replacing it with metX and metY genes, successfully restored L-methionine production ability, by converting its methionine biosynthesis pathway from trans—to direct-sulfurylation (FIGS. 3B and 3C). The invention is further based on the surprising finding, that insertion of metX and metY genes, from several bacteria strains, comprising Cyclobacterium marinum (CM) and Deinococcus geothermalis (DG), are more effective in converting E. coli to an effective L-methionine producer. In some embodiments it was discovered that additional inactivation of the metJ gene, overexpression of the yjeH gene encoding a L-methionine exporter, or both, further improved L-methionine production ability (FIGS. 4 and 5).

According to a first aspect, there is provided a microorganism comprising an exogenous nucleic acid molecule or a plurality thereof, encoding homoserine O-acetyltransferase (HAT) and O-acetylhomoserine sulfhydrylase (OAHS). In some embodiments, the microorganism further comprises inactive genes encoding homoserine O-succinyltransferase (HST) and cystathionine gamma-synthase (CgS) genes. In some embodiments, HAT is encoded by the metX gene. In some embodiments, OAHS is encoded by the metY gene. In some embodiments, HST is encoded by the metA gene. In some embodiments, CgS is encoded by the metB gene. In some embodiments, the microorganism comprises a bacterial cell. In some embodiments, the bacterial cell is characterized by increased amino acid production, secretion, or both, compared to a wild type, genetic reference or background, control bacterial cell. In some embodiments, a wild type, genetic reference or background, control bacterial cell comprises: an active metA gene, an active metB gene, an active metJ gene, or any combination thereof. In some embodiments, a wild type, genetic reference or background, control bacterial cell is devoid of metX gene, metY gene, or both. In some embodiments, a wild type, genetic reference or background, control bacterial cell comprises inactive metX gene, inactive metY gene, or both.

According to another aspect, there is provided a bacterial cell comprising: an exogenous nucleic acid molecule or a plurality thereof, encoding homoserine O-acetyltransferase (metX) and O-acetylhomoserine sulfhydrylase (metY).

In some embodiments, the bacterial cell further comprises inactive homoserine O-succinyltransferase (metA) gene and/or inactive cystathionine gamma-synthase (metB) gene and/or inactive metJ methionine repressor gene. In some embodiments, the bacterial cell further comprises inactive homoserine O-succinyltransferase (metA) gene, inactive cystathionine gamma-synthase (metB) gene, and inactive metJ methionine repressor gene.

For simplicity of the invention description, in several parts of the application, the terms: HAT and metX are interchangeably used. This also applies to OAHS and metY, HST and metA and to CgS and metB.

In some embodiments, the bacterial cell disclosed herein comprises an exogenous nucleic acid molecule or a plurality thereof, encoding homoserine O-acetyltransferase (HAT).

Homoserine O-acetyltransferase (EC no. 2.3.1.31) is an enzyme that belongs to the family of transferases and catalyzes the chemical reaction acetyl-CoA+L-homoserine→CoA+O-acetyl-L-homoserine. The systematic name of this enzyme class is acetyl-CoA: L-homoserine O-acetyltransferase. Other names include homoserine acetyltransferase, homoserine transacetylase, homoserine-O-transacetylase, and L-homoserine O-acetyltransferase. This enzyme participates in methionine metabolism and sulfur metabolism. In some embodiments, HAT enzyme participates in methionine metabolism and sulfur metabolism. In some embodiments, HAT enzyme is encoded by the metX gene.

In some embodiments, the bacterial cell disclosed herein comprises an exogenous nucleic acid molecule or a plurality thereof, encoding O-acetylhomoserine sulfhydrylase (OAHS).

O-acetylhomoserine sulfhydrolase (OAHS) or O-acetylhomoserine aminocarboxypropyltransferase (EC no. 2.5.1.49) is an enzyme that belongs to the family of transferases and catalyzes the chemical reaction O-acetyl-L-homoserine+methanethiol→L-methionine+acetate. The systematic name of this enzyme class is O-acetyl-L-homoserine: methanethiol 3-amino-3-carboxypropyltransferase. Other names include O-acetyl-L-homoserine acetate-lyase (adding methanethiol), O-acetyl-L-homoserine sulfhydrolase, O-acetylhomoserine (thiol)-lyase, O-acetylhomoserine sulfhydrolase, and methionine synthase. In some embodiments, OAHS enzyme participates in methionine metabolism and cysteine metabolism. In some embodiments, OAHS enzyme is encoded by the metY gene.

In some embodiments, the bacterial cell disclosed herein comprises an inactive homoserine O-succinyltransferase (HST).

Homoserine O-succinyltransferase (EC no. 2.3.1.46), is an enzyme that belongs to the family of transferases and catalyzes the chemical reaction succinyl-CoA+L-homoserine→CoA+O-succinyl-L-homoserine. Other names include: succinyl-CoA: L-homoserine O-succinyltransferase, homoserine O-transsuccinylase, or homoserine succinyltransferase. In some embodiments, HST enzyme participates in methionine metabolism and sulfur metabolism. In some embodiments, HST is encoded by the metA gene. In some embodiments, HST catalyzes the activation of homoserine. In some embodiments, activation of homoserine by succinylation allows the trans-sulfurylation to occur. Several bacteria strains were reported to express HST, particularly proteobacteria. In some embodiments, the bacterial cell that expresses HST comprises at least one of: proteobacteria and actinobacteria. In some embodiments, the proteobacteria comprises gammaproteobacteria or zetaproteobacteria. In some embodiments, gammaprobacteria comprises E. coli.

In some embodiments, the bacterial cell disclosed herein comprises an inactive cystathionine gamma-synthase (CgS).

Cystathionine gamma-synthase, (EC no. 2.5.1.48) is an enzyme that belongs to the family of transferases and catalyzes the formation of cystathionine from cysteine and an activated derivative of homoserine (e.g. 04-succinyl-L-homoserine+L-cysteine→L-cystathionine+succinate.

In some embodiments, the activated substrate of CgS is 04-succinyl-L-homoserine or 04-acetyl-L-homoserine. The systematic name of CgS class is 04-succinyl-L-homoserine: L-cysteine S-(3-amino-3-carboxypropyl) transferase. Other names include O-succinyl-L-homoserine succinate-lyase (adding cysteine), O-succinylhomoserine (thiol)-lyase, homoserine O-transsuccinylase, O-succinylhomoserine synthase, O-succinylhomoserine synthetase, cystathionine synthase, cystathionine synthetase, homoserine transsuccinylase, 4-O-succinyl-L-homoserine: L-cysteine, and S-(3-amino-3-carboxypropyl) transferase. In some embodiments, CgS enzyme participates in metabolic pathways such as: methionine metabolism, cysteine metabolism, selenoamino acid metabolism, and sulfur metabolism. In some embodiments, CgS enzyme is encoded by metB gene. In some embodiments, CgS. is encoded by metB in E. coli. In some embodiments, CgS is encoded by metI in B. subtilis. In some embodiments, metB is naturally present in actinobacteria and/or proteobacteria, comprising: alphaproteobacteria, and gammaproteobacteria. In some embodiments, gammaproteobacterial comprises E. coli.

In some embodiments, there is provided a bacterial cell comprising: (i) an exogenous nucleic acid molecule or a plurality thereof, encoding homoserine O-acetyltransferase (metX) and O-acetylhomoserine sulfhydrylase (metY), and wherein the cell is devoid of homoserine O-succinyltransferase (metA) and/or cystathionine gamma-synthase (metB) genes.

In some embodiments, there is provided a bacterial cell comprising: (i) an exogenous nucleic acid molecule or a plurality thereof, encoding homoserine O-acetyltransferase (metX) and O-acetylhomoserine sulfhydrylase (metY), and wherein the cell comprises non-functional homoserine O-succinyltransferase (metA) and/or cystathionine gamma-synthase (metB) genes.

In some embodiments, non-functional genes, e.g., metA and/or metB, as disclosed herein, comprise a premature stop codon (‘nonsense mutation’), a frame shift, a missense mutation, or any combination thereof, rendering a protein product encoded from a gene non-functional. In some embodiments, non-functional comprises at least partially non-functional or completely non-functional.

In some embodiments, the microorganism disclosed herein is a cell that naturally (i.e. its counterpart wild-type) utilizes the trans-sulfurylation pathway to produce L-methionine.

As used herein, the term “trans-sulfurylation pathway” refers to a metabolic pathway involving the interconversion of cysteine and homocysteine through the intermediate cystathionine. In some embodiments, the trans-sulfurylation pathway is the forward trans-sulfurylation pathway. In some embodiments, the product of the forward trans-sulfurylation pathway comprises L-homocysteine.

In some embodiments, the forward pathway is present in several bacteria, comprising Escherichia coli (E. coli) and Bacillus subtilis (B. subtilis).

As used herein, the forward trans-sulfurylation pathway refers to the transfer of a thiol group from cysteine to homocysteine, by the γ-replacement of the acetyl or succinyl group of a homoserine with cysteine to form cystathionine. Cystathionine is cleaved by β-elimination of its homocysteine portion. In some embodiments, the transfer from cystathionine to homocysteine is catalyzed by cystathionine β-lyase (CbL). In some embodiments, the CbL is encoded by metC gene. In some embodiments, the trans-sulfurylation pathway comprises two steps: (i) production of cystathionine and succinate from O-succinylhomoserine and cysteine; and, (ii) cleavage of cystathionine to form homocysteine, pyruvate and ammonia. In some embodiments, step (i) is catalyzed by cystathionine γ-synthase (CgS). In some embodiments, CgS is encoded by metB. In some embodiments, step (ii) is catalyzed by cystathionine β-lyase (CbL). In some embodiments, CbL is encoded by metC. In some embodiments, the production of homocysteine through trans-sulfuration allows the conversion of this intermediate to methionine, through a methylation reaction carried out by methionine synthase. In some embodiments, the trans-sulfurylation pathway comprises the catalytic activity of CgS and/or CbL. In some embodiments, the trans-sulfurylation pathway comprises the catalytic activity of the enzymes encoded by metB and/or metC.

In some embodiments, the microorganism disclosed herein is a cell that naturally (i.e., its wild-type counterpart) comprises at least one of: HST (metA) and CgS (metB). In some embodiments, the microorganism disclosed herein is a cell that its wild-type counterpart comprises HST (metA) and CgS (metB). In some embodiments, the microorganism comprises a bacterial cell. In some embodiments, the bacterial cell disclosed herein is a bacterial cell that its counterpart wild-type comprises HST (metA) and CgS (metB). In some embodiments, the bacterial cell is selected from: actinobacteria and proteobacteria. In some embodiments, the proteobacteria is selected from: gammaproteobacterial, alphaproteobacterial and zetaproteobacteria. In some embodiments, gammaproteobacterial cell comprises E. coli. In some embodiments, the bacterial cell disclosed herein is selected from E. coli and B. subtilis.

In some embodiments, there is provided a bacterial cell comprising an exogenous nucleic acid molecule or a plurality thereof comprising metX and metY genes. In some embodiments, the exogenous nucleic acid molecule or a plurality thereof comprises metX. In some embodiments, the exogenous nucleic acid molecule or a plurality thereof comprises metY. In some embodiments at least one of the genes: metA and metB, is inactivated. In some embodiments, metA and metB are inactivated.

In some embodiments, the bacteria disclosed herein comprises an exogenous nucleic acid molecule or a plurality thereof, comprising metX, and at least one inactivated gene selected from: metA and metB. In some embodiments, the exogenous nucleic acid molecule or a plurality thereof, comprises metY, and at least one of the genes selected from: metA and metB, is inactivated. In some embodiments, the exogenous nucleic acid molecule or a plurality thereof comprises metX and metY, and at least one of the genes selected from: metA and metB is inactivated. In some embodiments, the exogenous nucleic acid molecule or a plurality thereof comprises metX and metY, and inactivated metA and metB genes.

In some embodiments, the bacterial cell further comprises an inactive MetJ gene and/or a protein product thereof. In some embodiments, MetJ is a protein that functions as a repressor for methionine synthesis. In some embodiments, MetJ is a homodimer that interacts with DNA bases via a ribbon-helix-helix (RHH) motif. In some embodiments, MetJ regulates the methionine synthesis by binding to the DNA operator site of a “Met box”, halting transcription of genes involved in methionine synthesis. In some embodiments, MetJ is transcribed by metJ gene. In some embodiments, the bacterial cell disclosed herein further comprises an inactive metJ gene.

In some embodiments, any one of the inactive genes is knocked out, knocked down, mutated, or chemically inhibited. As used herein, the terms “knocked out” and “knockout” are used interchangeably, and refer to a gene that is inactive. In some embodiments, the inactivation is by a genetic technique. In some embodiments, the genetic technique comprises a method in which the gene, or at least part of it (e.g. the operon or the protein coding sequence), is taken out from the organism's genome.

Methods for generation of knocked out genes are well known in the art. Non-limiting examples comprise: homologous recombination, site-specific nucleases, zinc finger nuclease, transcription activator-like effector nuclease (TALENTS), and clustered regularly interspaced short palindromic repeats (CRISPR)/Cas9.

In some embodiments, any one of the inactive genes is knocked down. As used herein, the term “knocked down”, or “knockdown”, refers to a condition in which the expression of the gene is reduced. In some embodiments, the transcription of the gene is reduced. In some embodiments, the translation of the gene is reduced. In some embodiments, the expression of the gene is reduced by at least 10%, at least 20%, at least 30%, at least 40%, at least 50%, at least 60%, at least 70%, at least 80%, at least 90%, at least 95%, or by at least 99%. Each embodiment refers to a separate embodiment of the invention. In some embodiments, the reduction can occur either through a genetic modification or by treatment with a reagent such as a short DNA or RNA oligonucleotide that has a nucleic acid sequence complementary to either gene or a mRNA transcript.

In some embodiments, the knocked down gene is achieved by a genetic modification or mutation, leading to gene silencing. In some embodiments, the knocked down gene is by a stable knockdown, referring to constituent reduced gene expression. In some embodiments, the gene reduced expression is caused by an oligonucleotide binding to mRNA or temporarily binding to a gene. In some embodiments, the knocked down gene is a transient knockdown referring to a temporary change in gene expression that does not modify the chromosomal DNA. In some embodiments, the transient knockdown, is achieved by at least one of: blocking or reducing transcription, degradation of the mRNA transcript, blocking or reducing mRNA translation, blocking or reducing the binding of pre-mRNA splicing sites, or nuclease cleavage sites used for maturation of other functional RNAs, including miRNA. In some embodiments, blocking or reducing transcription can be achieved by small interfering RNA (siRNA)) or RNase-H dependent antisense. Methods for generation of knockdown genes are known in the art, comprising RNA interference, CRISPRs, and TALENs.

In some embodiments, the bacterial cell comprises one or more inhibitory nucleic acid sequences being complementary to any one of: metA, metB, and metJ genes. In some embodiments, the inhibitory nucleic acid molecule comprises at least one of: small interfering RNA (siRNA), microRNA (miRNA), or piwi-associated RNA (piRNA). In some embodiment the bacterial cell comprises a ribozyme (i.e., a catalytic RNA molecule that cleaves mRNA molecule).

In some embodiments inactivation of a gene is by a mutation. In some embodiments the mutation comprises a nucleic acid sequence comprising a premature stop codon compared to a wildtype form of the gene. As used herein, the terms “premature stop codon” and “premature termination codon” (PTC) are used interchangeably. In some embodiments, PTC is derived from a nucleotide mutation that converts a canonical triplet nucleotide codon into one of three stop codons, (e.g., TAG, TGA, or TAA). In some embodiments, PTC results in the loss of protein expression.

In some embodiments, the gene is inactivated by a chemical inhibition. In some embodiments, the chemical inhibition is by a small molecule (e.g., by a small organic molecule). In some embodiments, the small molecule inhibits gene transcription. In some embodiments, the small molecule inhibits mRNA translation. The term “small molecule” or micromolecule refers to a low molecular weight (≤1000 daltons) organic compound that can regulate a biological process.

In some embodiments, inactivation of a gene, or reduced activation of a gene, is induced, and dependent upon addition of an inducer supplementary. In other embodiments, inactivation of a gene, or reduced activation of a gene is constituent.

In some embodiments, the amino acid sequence of at least one homoserine O-acetyltransferase (HAT), encoded by metX, and O-acetylhomoserine sulfhydrylase (OAHS), encoded by metY, is selected from: leptospira interrogans (LI), Corynebacterium glutamicum (CG), deinococcus geothermalis (DG), sulfurimonas autotrophica (SA), Cyclobacterium marinum (CM) and Thermus thermophilus (TT).

In some embodiments, at least one of: HAT and OAHS, is from the phylum spirochaetota. In some embodiments, at least one of: HAT and OAHS, is from the class spirochaetia. In some embodiments, at least one of: HAT and OAHS, is from the order leptospirales. In some embodiments, at least one of: HAT and OAHS, is from the family leptospiraceae. In some embodiments, at least one of: HAT and OAHS, is from the genus leptospira. In some embodiments, at least one of: HAT and OAHS, is from the species leptospira interrogans (LI).

In some embodiments, at least one of: HAT and OAHS, is from the phylum actinomycetota. In some embodiments, at least one of: HAT and OAHS, is from the class actinomycetia. In some embodiments, at least one of: HAT and OAHS, is from the order mycobacteriales. In some embodiments, at least one of: HAT and OAHS, is from the family corynebacteriaceae. In some embodiments, at least one of: HAT and OAHS, is from the genus Corynebacterium. In some embodiments, at least one of: HAT and OAHS, is from the species Corynebacterium glutamicum (CG).

In some embodiments, at least one of: HAT and OAHS, is from the phylum deinococcota. In some embodiments, at least one of: HAT and OAHS, is from the class deinococci. In some embodiments, at least one of: HAT and OAHS, is from the order deinococcales. In some embodiments, at least one of: HAT and OAHS, is from the family deinococcaceae. In some embodiments, at least one of: HAT and OAHS, is from the genus deinococcus. In some embodiments, at least one of: HAT and OAHS, is from the species deinococcus geothermalis (DG).

In some embodiments, at least one of: HAT and OAHS, is from the phylum campylobacterota. In some embodiments, at least one of: HAT and OAHS, is from the class campylobacteria. In some embodiments, at least one of: HAT and OAHS, is from the order campylobacterales. In some embodiments, at least one of: HAT and OAHS, is from the family helicobacteraceae. In some embodiments, at least one of: HAT and OAHS, is from the genus sulfurimonas. In some embodiments, at least one of: HAT and OAHS, is from the species sulfurimonas autotrophica (SA).

In some embodiments, at least one of: HAT and OAHS, is from the phylum bacteroidota. In some embodiments, at least one of: HAT and OAHS, is from the class cytophagia. In some embodiments, at least one of: HAT and OAHS, is from the order cytophagales. In some embodiments, at least one of: HAT and OAHS, is from the family cyclobacteriaceae. In some embodiments, at least one of: HAT and OAHS, is from the genus cyclobacterium. In some embodiments, at least one of: HAT and OAHS, is from the species cyclobacterium marinum (CM).

In some embodiments, at least one of: HAT and OAHS, is from the phylum deinococcota. In some embodiments, at least one of: HAT and OAHS, is from the class deinococci. In some embodiments, at least one of: HAT and OAHS, is from the order thermals. In some embodiments, at least one of: HAT and OAHS, is from the family thermaceae. In some embodiments, at least one of: HAT and OAHS, is from the genus thermus. In some embodiments, at least one of: HAT and OAHS, is from the Thermus thermophilus (TT).

In some embodiments, the bacterial cell comprising an exogenous nucleic acid molecule or a plurality thereof, encoding homoserine O-acetyltransferase (HAT), encoded by metX, comprising the amino acid sequence:

(SEQ ID NO: 1)
MNETGSIGIIETKYAEFKELILNNGSVLSPVVIAYETYGTLSSSK
NNAILICHALSGDAHAAGYHSGSDKKPGWWDDYIGPGKSFDTNQY
FIICSNVIGGCKGSSGPLSIHPETSTPYGSRFPFVSIQDMVKAQK
LLVESLGIEKLFCVAGGSMGGMQALEWSIAYPNSLSNCIVMASTA
EHSAMQIAFNEVGRQAILSDPNWKNGLYDENSPRKGLALARMVGH
ITYLSDDKMREKFGRNPPRGNILSTDFAVGSYLIYQGESFVDRFD
ANSYIYVTKALDHYSLGKGKELTAALSNATCRFLVVSYSSDWLYP
PAQSREIVKSLEAADKRVFYVELQSGEGHDSFLLKNPKQIEILKG
FLENPN.

In some embodiments, homoserine O-acetyltransferase (HAT encoded by metX), comprises the amino acid sequence:

(SEQ ID NO: 2)
MPTLAPSGQLEIQAIGDVSTEAGAIIKNAEIAYHRWGEYRVDKEG
RSNVVLIEHALTGDSNAADWWADLLGPGKAINTDIYCVICTNVIG
GCNGSTGPGSMHPDGNFWGNRFPATSIRDQVNAEKQFLDALGITT
VAAVLGGSMGGARTLEWAAMYPEIVGAAAVLAVSARASAWQIGIQ
SAQIKAIENDHHWHEGNYYESGCNPATGLGAARRIAHLTYRGELE
IDERFGTKAQKNENPLGPYRKPDQRFAVESYLDYQADKLVQRFDA
GSYVLLTDALNRHDIGRDRGGLNKALESIKVPVLVAGVDTDILYL
YHQQEHLSRNLGNLLAMAKIVSPVGHDAFLTESRQMDRIVRNFFS
LISPDENNPSTYIEFYI.

In some embodiments, homoserine O-acetyltransferase (HAT), encoded by metX, comprises the amino acid sequence:

(SEQ ID NO: 3)
MTALISQPDLLPPPAPERCPPQQTARLFRETPLLLDCGQVVQDVR
VAYHTYGTPSDHAILVLHALTGTSAVHEWWPDFLGEGKPLDPTRD
YIVCANVLGGCAGSTGPAELPRVNGEDPPLTLRDMARVGRALLEE
LGVRRVSVIGASMGGMLAYAWLLECPDLVDRAVIIGAPARHSPWA
IGLNTAARNAIRAAPGGEGLKVARQIAMLSYRSPESFALTQSGWG
TRRPGTPDITTYLEHQGEKLSTRFCERSYLALTGAMDRFQPTDAE
LRSIRVPVLVVGISSDVLYPPAEVRTYAGLLPRGQYLELQSPHGH
DAFLIDPQGLPEAAAAFLHGA.

In some embodiments, homoserine O-acetyltransferase (HAT), encoded by metX, comprises the amino acid sequence:

(SEQ ID NO: 4)
MSLNLQTYTEHFTNPLYLESGRILEPYDITYETYGTMNEDKSNVV
VVCHALTGSHHAAGLYEDETKPGWWDGFIGSGKAIDTDKYFVICS
NVIGSCFGSTGPMSLQHPYQEPYRYKFPVVSIKDMVKAQRILFDR
LDIHRVHAIVGGSMGGMQALQFAIHYPNFANKIIALATTHATQPW
AIAFNKVAQESILNDPDFKQGYYDPDLLKEQGLSGMAVGRMAGHI
SFLSHESMREKFGRDYKLTDGLYELFGKFQVESYLEYNGYNFTKW
FDPLAYLYITKAINIYDLSRGFDSLAEALKRVTSALYLVSFKNDL
LFKNFEMKEIADELDKIGNKNHSYIDVKSDYGHDAFLVELNKFEN
HVKDALNG.

In some embodiments, homoserine O-acetyltransferase (HAT), encoded by metX, comprises the amino acid sequence:

(SEQ ID NO: 5)
MNLQSPHLTIEMTQEIFYCQEALSLESGESFPEFQLSFTTQGQLNANKD
NVIWVLHALTGDANPHEWWSGLIGEDKFFDPSKYFIVCANFLGSCYGST
QPLSNNPNNGKPYYYDFPNITTRDIASALDKLRIHLGLEKINTVIGGSL
GGQVGLEWAVSLGEKLENAIIVASNAKASPWIIGFNETQRMAIESDSTW
GKTQPEAGKKGLETARAIGMLSYRHPMTFLQNQSETEEKRDDFKISSYL
RYQGLKLANRFNAMSYWILSKAMDSHDIGRGRGGTPVALSNIKCKVLSI
GVDTDILFTSEESRYISKHVPKGTYREISSIYGHDAFLIEYEQLQYILK
SFYLENNG.

In some embodiments, homoserine O-acetyltransferase (HAT), encoded by metX, comprises the amino acid sequence:

(SEQ ID NO: 6)
MSEIALEAWGEHEALLLKPPRSPLSIPPPKPRTAVLFPRREGFYTELGG
YLPEVRLRFETYGTLSRRRDNAVLVFHALTGSAHLAGTYDEETFRSLSP
LEQAFGREGWWDSLVGPGRILDPALYYVVSANHLGSCYGSTGPLSLDPH
TGRPYGRDFPPLTIRDLARAQARLLDHLGVEKAIVIGGSLGGMVALEFA
LMYPERVKKLVVLAAPARHGPWARAFNHLSRQAILQDPEYQKGNPAPKG
MALARGIAMMSYRAPEGFEARWGAEPELGETYLDYQGEKFLRRFHAESY
LVLSRAMDTHDVGRGRGGVEEALKRLRAIPSLFVGIDTDLLYPAWEVRQ
AAKAAGARYREIKSPHGHDAFLIETDQVEEILDAFLP.

In some embodiments, homoserine O-acetyltransferase (HAT), encoded by metX, comprises the amino acid sequence:

(SEQ ID NO: 50)
PTLAPSGQLEIQAIGDVSTEAGAIIKNAEIAYHRWGEYRVDKEGRSNVV
LIEHALTGDSNAADWWADLLGPGKAINTDIYCVICTNVIGGCNGSTGPG
SMHPDGNFWGNRFPATSIRDQVNAEKQFLDALGITTVAAVLGGSMGGAR
TLEWAAMYPEIVGAAAVLAVSARASAWQIGIQSAQIKAIENDHHWHEGN
YYESGCNPATGLGAARRIAHLTYRGELEIDERFGTKAQKNENPLGPYRK
PDQRFAVESYLDYQADKLVQRFDAGSYVLLTDALNRHDIGRDRGGLNKA
LESIKVPVLVAGVDTDILYPYHQQEHLSRNLGNLLAMAKIVSPVGHDAF
LTESRQMDRIVRNFFSLISPDENNPSTYIEFYI.

In some embodiments, the bacterial cell comprises an exogenous nucleic acid molecule or a plurality thereof encoding an amino acid sequence selected from SEQ ID Nos: 1-6, and 50.

In some embodiments, the bacterial cell comprises an exogenous nucleic acid molecule or a plurality thereof encoding the amino acid sequence set forth in SEQ ID NO: 3 or SEQ ID NO: 5.

In some embodiments, HAT sequence from LI comprises SEQ ID NO: 1. In some embodiments, HAT sequence from CG comprises SEQ ID NO: 2. In some embodiments, HAT sequence from DG comprises SEQ ID NO: 3. In some embodiments, HAT sequence from SA comprises SEQ ID NO: 4. In some embodiments, HAT sequence from CM comprises SEQ ID NO: 5. In some embodiments, HAT sequence from TT comprises SEQ ID NO: 6.

In some embodiments, the bacterial cell comprises a nucleic acid or a plurality thereof, encoding a functional analog of any one of SEQ IN NOs: 1-6, and 50. In some embodiments, the bacterial cell comprises a nucleic acid molecule or a plurality thereof, encoding a functional analog of SEQ IN Nos: 3 or 5. The term “analog” as used herein, refers to a polypeptide that is similar, but not identical, to the polypeptide of HAT, and that is still capable of catalyzing the reaction of homoserine O-acetyltransferase. An analog may have deletions or mutations that result in an amino acids sequence that is different than the amino acid sequence of the polypeptide of the invention. It should be understood that all analogs of the polypeptide of the invention would still be capable of catalyzing the formation of acetyl-L-homoserine from L-homoserine. Further, an analog may be analogous to a fragment of the polypeptide of the invention, however, in such a case the fragment must comprise at least 100 consecutive amino acids of the polypeptide of the invention.

In some embodiments, an analog to the polypeptide disclosed herein comprises an amino acid sequence with at least 60%, at least 65%, at least 70%, at least 75%, at least 80%, at least 85%, at least 90%, at least 95%, at least 99% homology to the amino acid sequence presented in SEQ ID NOs: 1-6, and 50. Each embodiment refers to a separate embodiment of the invention.

In some embodiments, the bacterial cell comprising an exogenous nucleic acid molecule or a plurality thereof, encoding O-acetylhomoserine sulfhydrylase (OAHS encoded by metY), comprises the amino acid sequence:

(SEQ ID NO: 7)
MPRNYKPETIALHGGQSPDPSTLSRAVPIYQTTSYVFKNTEHAAKLFGL
QEFGNIYTRIMNPTTDVLEQRIAALEGGVAALATASGQAAETLALLNIV
EAGQEIVASSSLYGGTYNLLHYTFPKLGIKVHFVDPSDPENFRKAVNDK
TRAFYAETLGNPKLDTLNLEAIAKVAHDSEVPLIIDNTLPSPYLVNPIE
HGADIVVHSLTKFLGGHGTSIGGIIVDSGKFNWGNGKFKNFTEPDPSYH
GLKFWEVFGKFEPFGGVNIAYIIKAKVQGLRDMGASISPFNAWQILQGV
ETLPLRMRKHSENALAVAEYLTKHTKVSWVNYPGLKMDKNYSLAKKYHK
KDLYGAILGFGIKGGAVEAKKFIDGLELFSLLANVGDAKSLVIHPASTT
HQQLTPEEQLSAGVTPDFVRLSVGLENIEDILFDLEEALKKV.

In some embodiments, O-acetylhomoserine sulfhydrylase (OAHS) encoded by metY, comprises the amino acid sequence:

(SEQ ID NO: 8)
MPKYDNSNADQWGFETRSIHAGQSVDAQTSARNLPIYQSTAFVFDSAEH
AKQRFALEDLGPVYSRLTNPTVEALENRIASLEGGVHAVAFSSGQAATT
NAILNLAGAGDHIVTSPRLYGGTETLFLITLNRLGIDVSFVENPDDPES
WQAAVQPNTKAFFGETFANPQADVLDIPAVAEVAHRNSVPLIIDNTIAT
AALVRPLELGADVVVASLTKFYTGNGSGLGGVLIDGGKFDWTVEKDGKS
VFPYFVTPDAAYHGLKYADLGAPAFGLKVRVGLLRDTGSTLSAFNAWAA
VQGIDTLSLRLERHNENAIKVAEFLNNHEKVEKVNFAGLKDSPWYATKE
KLGLKYTGSVLTFEIKGGKDEAWAFIDALKLHSNLANIGDVRSLVVHPA
TTTHSQSDEAGLARAGVTQSTVRLSVGIETIDDIIADLEGGFAAI.

In some embodiments, O-acetylhomoserine sulfhydrylase (OAHS) encoded by metY, comprises the

(SEQ ID NO: 9)
MASNTLHFETLQVHAGQHPDPATGAQAVPIYATNAYVFESPEHAADLFG
LRAFGNIYSRIMNPTNAVLEERIAALEGGVGALAVASGHAAQFLAITTV
AQAGDNIVSTPNLYGGTVNQFRVTLRRLGIEVRFTSKDERPEEFAALID
DRTRAVYLETLGNPALNVPDFEGIAEVAHARGVAVFVDNTFGAGGYYCQ
PLRHGADVVLHSASKWIGGHGNGIGGLLVDGGTFDWGNGRYPLLTEPSP
SYHGLSFWEAFGEGNALGLPNIAFITRARTEGLRDLGPTLAPQQAWQFL
QGVETLSLRAERHAQNALALASWLSGHPDVSRVTYPGLSNHPHYDRAQT
YLPRGAGAVLTFELRGGRAAGEAFIGAVRLAQHVANVGDTRTLVIHPAS
TTHSQLDEAAQAAAGVTPGLVRVSVGIEHIDDIREDFAQALATALVDAE
GA.

In some embodiments, O-acetylhomoserine sulfhydrylase (OAHS) encoded by metY, comprises the

(SEQ ID NO: 10)
MDLQTLALHAGYEKDSQRTMAVPIYQTTAYEFRDVEHAANLFALKELGN
IYTRLNNPTTDVFEKRFTALEGGEAAIATASGMSAIFFALANAAQAGDN
IICANQLYGGSLTLSTHTLKRFGIEARFFDVHKPQQIEVLIDEKTKVIF
FESLTNPSIDVADIEALTAIANKHNILTIVDNTVATPVLCRPFEFGADI
TVHSASKYTTGQGLAIGGILVERKGLVDKLKNNPRYSHFNEPDASYHGL
VYTDTGLPPYTLRARLSLLRDLGAVVSPFNSWLFIQGIETLSLRMKEHS
KNALMLAEFLESHKKVKKVNYPGLKSNANYKNAQKYFQDGLCSGLLSFE
VEDFDEATKIVDATKLYSLVVNIGDSKSIITHPASTTHQQLSHEELIAC
GVPEGLIRISCGLESVKDLIEDLKQALEA.

In some embodiments, O-acetylhomoserine sulfhydrylase (OAHS) encoded by metY, comprises the amino acid sequence:

(SEQ ID NO: 11)
MSKNYRFETLQVHGGQEVDPTTNSRAVPIYQTSSYVFNSAEHGANLFAL
KEFGNIYTRIMNPTSDVFEKRMAALEGGVAAVATASGQAAQFLALNNFL
SVGDNFVTSPFLYGGSYNQFKVSFKRIGIEARFAKSDKVDDLAAEINDK
TKAIYVETIGNPEFNVPDFEAIAALAKKHDIPLVVDNTFGAGGYLCQPI
KHGANIVTSSATKWIGGHGTSIGGIIVDGGNYNWGNGKFPQFSEPSEGY
HGLNFWETFGDNNPLGLPNIAFAIRARVEGLRDFGPAISPFNSFLLLQG
LETLSLRVQRTVDNALELAKWLEAHPKVKSVNYPGLTNSPYHATAKKYL
THGFGGVLSFEIEGDKETASNFINNLELISHLANVGDAKTLIIQPSATT
HQQLSDEAQIAAGVTPSLLRISSGIEHIEDLKADLTAAFDKI.

In some embodiments, O-acetylhomoserine sulfhydrylase (OAHS) encoded by metY, comprises the

(SEQ ID NO: 12)
MRFETLQLHAGYEPEPTTLSRQVPIYPTTSYVFKSPEHAANLFALKEFG
NIYSRIMNPTVDVLEKRLAALEGGKAALATASGHAAQFLALTTLAQAGD
NIVSTPNLYGGTFNQFKVTLKRLGIEVRFTSREERPEEFLALTDERTRA
SWVESIGNPALNIPDLEALAQAAREKGVALIVDNTFGMGGYLLRPLAWG
AALVTHSLTKWVGGHGAVIAGAIVDGGSFPWEGGRYPLLTEPQPGYHGL
RLTEAFGELAFIVKARVDGLRDQGQALGPFEAWVVLLGMETLSLRAERH
VENTLHLAHWLLEQPQVAWVNYPGLPHHPHHDRAQKYFKGKPGAVLTFG
LKGGYEAAKRFISRLKLISHLANVGDTRTLAIHPASTTHSQLSPEEQAQ
AGVSPEMVRLSVGLEHVEDLKAELKEALA.

In some embodiments, the bacterial cell comprises an exogenous nucleic acid molecule or a plurality thereof, encoding O-acetylhomoserine sulfhydrylase (OAHS) encoded by metY, comprising the amino acid sequence selected from SEQ ID NOs: 7-12.

In some embodiments, the bacterial cell comprises an exogenous nucleic acid molecule or a plurality thereof encoding O-acetylhomoserine sulfhydrylase (OAHS) encoded by metY, comprising the amino acid sequence set forth in SEQ ID NO: 9 or SEQ ID NO: 11.

In some embodiments, the bacterial cell comprises an exogenous nucleic acid molecule or a plurality thereof, encoding SEQ ID NO: 1, and SEQ ID NO: 7. In some embodiments, the bacterial cell comprises an exogenous nucleic acid molecule or a plurality thereof, encoding SEQ ID NO: 2, and SEQ ID NO: 8. In some embodiments, the bacterial cell comprises an exogenous nucleic acid molecule or a plurality thereof, encoding SEQ ID NO: 3, and SEQ ID NO: 9. In some embodiments, the bacterial cell comprises an exogenous nucleic acid molecule or a plurality thereof, encoding SEQ ID NO: 4, and SEQ ID NO: 10. In some embodiments, the bacterial cell comprises an exogenous nucleic acid molecule or a plurality thereof, encoding SEQ ID NO: 5, and SEQ ID NO: 11. In some embodiments, the bacterial cell comprises an exogenous nucleic acid molecule or a plurality thereof, encoding SEQ ID NO: 6, and SEQ ID NO: 12.

In some embodiments, OAHS sequence from LI comprises SEQ ID NO: 7. In some embodiments, OAHS sequence from CG comprises SEQ ID NO: 8. In some embodiments, OAHS sequence from DG comprises SEQ ID NO: 9. In some embodiments, OAHS sequence from SA comprises SEQ ID NO: 10. In some embodiments, OAHS sequence from CM comprises SEQ ID NO: 11. In some embodiments, OAHS sequence from TT comprises SEQ ID NO: 12.

In some embodiments, the bacterial cell comprises a nucleic acid or a plurality thereof, encoding a functional analog of any one of SEQ IN NOs: 7-12. In some embodiments, the bacterial cell comprises a nucleic acid molecule or a plurality thereof, encoding a functional analog set forth in SEQ IN Nos: 9 or 11. The term “analog” as used herein, refers to a polypeptide that is similar, but not identical, to the polypeptide of OAHS, and that still is capable of catalyzing the reaction of O-acetylhomoserine sulfhydrylase. An analog may have deletions or mutations that result in an amino acids sequence that is different than the amino acid sequence of the polypeptide of the invention. It should be understood that all analogs of the polypeptide of the invention would still be capable of catalyzing the formation of L-homocysteine. Further, an analog may be analogous to a fragment of the polypeptide of the invention, however, in such a case the fragment must comprise at least 100 consecutive amino acids of the polypeptide of the invention.

In some embodiments, an analog to the polypeptide sequence disclosed herein comprises an amino acid sequence with at least 60%, at least 65%, at least 70%, at least 75%, at least 80%, at least 85%, at least 90%, at least 95%, at least 99% homology to the amino acid sequence presented in SEQ ID NOs: 7-12. Each embodiment refers to a separate embodiment of the invention.

In some embodiments, the bacterial cell comprises an exogenous nucleic acid molecule or a plurality thereof, comprising a nucleic acid sequence being codon optimized for expression in the bacterial cell. As used herein, the term “codon optimized” describes a sequence that encodes identical amino acids to those encoded by a non-optimized codon sequence (synonymous codon), however, at least one of: translation rate of the codon optimized sequence, protein product amount, duration of protein structure stability, or any combination thereof, is increased, compared to the non-optimized codon. An ordinary skill in the art will know how to optimize a codon sequence for its expression in the desired cell, using a codon optimization gene engineering tool, comprising, but not limited to, algorithms that analyze codon optimization based on the codon frequencies in the desired cell/species. In some embodiments, increased one of: translation rate, protein product amount, and duration of structure stability, is by at least by 30%.

In some embodiments, the bacterial cell comprises a nucleic acid sequence being codon optimized for expression in a bacterial cell that naturally (or its wildtype counterpart) uses the forward trans-sulfurylation pathway for production of L-methionine. In some embodiments, the bacterial cell comprises a nucleic acid sequence being codon optimized for expression in E. coli.

In some embodiments, the bacterial comprises an exogenous nucleic acid molecule, of metX or metX optimized codon.

In some embodiments, the bacterial cell comprises an exogenous nucleic acid molecule, comprising the nucleotide sequence:

(SEQ ID NO: 13)
ATGAACGAGACCGGTAGCATCGGCATCATTGAAACCAAGTACGCGGAGT
TCAAAGAACTGATCCTGAACAACGGTAGCGTGCTGAGCCCGGTGGTTAT
TGCGTACGAGACCTATGGCACCCTGAGCAGCAGCAAGAACAACGCGATC
CTGATTTGCCATGCGCTGAGCGGTGATGCGCATGCGGCGGGTTATCACA
GCGGCAGCGATAAGAAACCGGGTTGGTGGGACGATTACATTGGTCCGGG
CAAGAGCTTCGACACCAACCAGTATTTTATCATTTGCAGCAACGTTATC
GGTGGCTGCAAAGGTAGCAGCGGCCCGCTGAGCATTCATCCGGAGACCA
GCACCCCGTATGGTAGCCGTTTCCCGTTTGTGAGCATCCAGGATATGGT
TAAGGCGCAAAAGCTGCTGGTTGAGAGCCTGGGTATTGAAAAACTGTTC
TGCGTTGCGGGTGGCAGCATGGGTGGCATGCAAGCGCTGGAATGGAGCA
TCGCGTATCCGAACAGCCTGAGCAACTGCATTGTTATGGCGAGCACCGC
GGAGCACAGCGCGATGCAGATCGCGTTTAACGAAGTTGGTCGTCAAGCG
ATTCTGAGCGACCCGAACTGGAAGAACGGCCTGTACGATGAGAACAGCC
CGCGTAAAGGTCTGGCGCTGGCGCGTATGGTGGGTCACATCACCTATCT
GAGCGACGATAAGATGCGTGAAAAATTCGGTCGTAACCCGCCGCGTGGC
AACATCCTGAGCACCGACTTTGCGGTTGGTAGCTACCTGATTTATCAGG
GCGAGAGCTTCGTGGACCGTTTTGATGCGAACAGCTACATCTATGTTAC
CAAGGCGCTGGACCACTACAGCCTGGGCAAGGGCAAAGAACTGACCGCG
GCGCTGAGCAACGCGACCTGCCGTTTCCTGGTGGTTAGCTACAGCAGCG
ATTGGCTGTATCCGCCGGCGCAAAGCCGTGAGATTGTGAAGAGCCTGGA
AGCGGCGGACAAACGTGTGTTCTACGTTGAGCTGCAAAGCGGTGAAGGC
CACGATAGCTTTCTGCTGAAGAACCCGAAACAAATCGAGATTCTGAAAG
GTTTTCTGGAAAACCCGAACTAA.

In some embodiments, the bacterial cell comprises an exogenous nucleic acid molecule, comprising

(SEQ ID NO: 14)
ATGAGCCTGAACCTGCAAACCTATACCGAGCACTTCACCAACCCGCTGT
ACCTGGAGAGCGGTCGTATCCTGGAACCGTACGATATTACCTACGAGAC
CTATGGCACCATGAACGAAGACAAGAGCAACGTGGTTGTGGTTTGCCAT
GCGCTGACCGGTAGCCACCATGCGGCGGGTCTGTATGAGGATGAAACCA
AACCGGGTTGGTGGGACGGCTTCATCGGTAGCGGCAAGGCGATTGACAC
CGATAAATACTTCGTGATTTGCAGCAACGTTATTGGTAGCTGCTTTGGT
AGCACCGGCCCGATGAGCCTGCAACACCCGTATCAAGAACCGTACCGTT
ATAAGTTCCCGGTGGTTAGCATCAAGGATATGGTGAAAGCGCAACGTAT
TCTGTTTGACCGTCTGGATATCCACCGTGTGCACGCGATTGTTGGTGGC
AGCATGGGTGGCATGCAGGCGCTGCAATTCGCGATCCACTACCCGAACT
TTGCGAACAAGATCATTGCGCTGGCGACCACCCATGCGACCCAGCCGTG
GGCGATCGCGTTCAACAAAGTGGCGCAAGAGAGCATTCTGAACGACCCG
GATTTTAAGCAGGGTTACTATGACCCGGATCTGCTGAAAGAACAAGGTC
TGAGCGGTATGGCGGTTGGTCGTATGGCGGGTCACATCAGCTTCCTGAG
CCACGAGAGCATGCGTGAAAAGTTTGGTCGTGATTATAAACTGACCGAC
GGTCTGTACGAGCTGTTCGGCAAGTTTCAGGTGGAGAGCTACCTGGAAT
ATAACGGCTACAACTTCACCAAGTGGTTTGATCCGCTGGCGTACCTGTA
CATCACCAAGGCGATCAACATCTACGATCTGAGCCGTGGTTTCGACAGC
CTGGCGGAGGCGCTGAAGCGTGTGACCAGCGCGCTGTACCTGGTTAGCT
TTAAAAACGACCTGCTGTTCAAGAACTTTGAGATGAAAGAAATCGCGGA
CGAACTGGATAAGATTGGTAACAAAAACCACAGCTATATCGACGTTAAA
AGCGATTACGGCCACGACGCGTTCCTGGTGGAGCTGAACAAGTTTGAAA
ACCACGTTAAAGACGCGCTGAACGGCTAA.

In some embodiments, the bacterial cell comprises an exogenous nucleic acid molecule, comprising the nucleotide sequence:

(SEQ ID NO: 15)
ATGCGTATCCAGCGTTACATTCTGATGACCGCGCTGATCAGCCAACCGG
ACCTGCTGCCGCCGCCGGCGCCGGAGCGTTGCCCGCCGCAGCAAACCGC
GCGTCTGTTCCGTGAAACCCCGCTGCTGCTGGACTGCGGTCAGGTGGTT
CAAGATGTGCGTGTTGCGTACCACACCTATGGCACCCCGAGCGACCATG
CGATCCTGGTGCTGCATGCGCTGACCGGCACCAGCGCGGTTCATGAGTG
GTGGCCGGATTTTCTGGGTGAAGGCAAGCCGCTGGACCCGACCCGTGAT
TATATTGTTTGCGCGAACGTTCTGGGTGGCTGCGCGGGTAGCACCGGTC
CGGCGGAGCTGCCGCGTGTGAACGGTGAAGACCCGCCGCTGACCCTGCG
TGATATGGCGCGTGTGGGTCGTGCGCTGCTGGAGGAACTGGGCGTTCGT
CGTGTGAGCGTTATTGGTGCGAGCATGGGTGGCATGCTGGCGTATGCGT
GGCTGCTGGAGTGCCCGGACCTGGTGGATCGTGCGGTTATCATTGGTGC
GCCGGCGCGTCACAGCCCGTGGGCGATTGGTCTGAACACCGCGGCGCGT
AACGCGATTCGTGCGGCGCCGGGTGGCGAGGGTCTGAAGGTTGCGCGTC
AGATCGCGATGCTGAGCTATCGTAGCCCGGAGAGCTTCGCGCTGACCCA
GAGCGGTTGGGGCACCCGTCGTCCGGGCACCCCGGACATTACCACCTAC
CTGGAGCACCAGGGTGAAAAACTGAGCACCCGTTTCTGCGAGCGTAGCT
ATCTGGCGCTGACCGGCGCGATGGACCGTTTTCAACCGACCGATGCGGA
ACTGCGTAGCATCCGTGTGCCGGTTCTGGTGGTTGGTATTAGCAGCGAT
GTGCTGTACCCGCCGGCGGAAGTGCGTACCTATGCGGGTCTGCTGCCGC
GTGGCCAGTACCTGGAACTGCAAAGCCCGCACGGCCATGATGCGTTCCT
GATCGATCCGCAGGGTCTGCCGGAAGCGGCGGCGGCGTTCCTGCACGGT
GCGTAA.

In some embodiments, the bacterial cell comprises an exogenous nucleic acid molecule, comprising the nucleotide sequence:

(SEQ ID NO: 16)
ATGAACCTGCAAAGCCCGCACCTGACCATCGAGATGACCCAGGAAATTT
TCTACTGCCAAGAGGCGCTGAGCCTGGAGAGCGGCGAGAGCTTCCCGGA
ATTTCAACTGAGCTTTACCACCCAGGGCCAACTGAACGCGAACAAGGAC
AACGTGATCTGGGTTCTGCACGCGCTGACCGGTGATGCGAACCCGCACG
AGTGGTGGAGCGGTCTGATCGGCGAAGACAAGTTCTTTGATCCGAGCAA
ATATTTCATTGTGTGCGCGAACTTTCTGGGTAGCTGCTACGGCAGCACC
CAGCCGCTGAGCAACAACCCGAACAACGGTAAACCGTACTATTACGACT
TCCCGAACATCACCACCCGTGACATTGCGAGCGCGCTGGATAAGCTGCG
TATCCACCTGGGCCTGGAGAAAATCAACACCGTGATTGGTGGCAGCCTG
GGTGGCCAAGTGGGTCTGGAATGGGCGGTTAGCCTGGGCGAGAAGCTGG
AAAACGCGATCATTGTTGCGAGCAACGCGAAAGCGAGCCCGTGGATCAT
TGGTTTTAACGAGACCCAGCGTATGGCGATCGAAAGCGATAGCACCTGG
GGCAAGACCCAACCGGAGGCGGGTAAGAAAGGCCTGGAAACCGCGCGTG
CGATTGGTATGCTGAGCTATCGTCACCCGATGACCTTCCTGCAAAACCA
AAGCGAGACCGAGGAAAAGCGTGACGATTTTAAAATCAGCAGCTATCTG
CGTTACCAGGGCCTGAAGCTGGCGAACCGTTTCAACGCGATGAGCTACT
GGATTCTGAGCAAAGCGATGGACAGCCACGATATTGGTCGTGGTCGTGG
TGGCACCCCGGTGGCGCTGAGCAACATCAAGTGCAAAGTGCTGAGCATC
GGTGTTGACACCGATATTCTGTTTACCAGCGAGGAAAGCCGTTATATTA
GCAAGCACGTTCCGAAAGGCACCTATCGTGAGATCAGCAGCATTTACGG
CCACGACGCGTTCCTGATCGAGTATGAACAGCTGCAATACATTCTGAAG
AGCTTCTACCTGGAAAACAACGGCTAA.

In some embodiments, the bacterial cell comprises an exogenous nucleic acid molecule, comprising

(SEQ ID NO: 17)
ATGCCGACCCTGGCGCCGAGCGGTCAGCTGGAGATCCAAGCGATTGGTG
ACGTTAGCACCGAGGCGGGCGCGATCATTAAGAACGCGGAAATTGCGTA
CCACCGTTGGGGTGAGTATCGTGTGGACAAAGAAGGCCGTAGCAACGTG
GTTCTGATCGAACACGCGCTGACCGGTGATAGCAACGCGGCGGACTGGT
GGGCGGATCTGCTGGGTCCGGGCAAGGCGATCAACACCGACATTTACTG
CGTTATCTGCACCAACGTGATCGGTGGCTGCAACGGCAGCACCGGTCCG
GGCAGCATGCACCCGGATGGTAACTTCTGGGGCAACCGTTTTCCGGCGA
CCAGCATTCGTGACCAGGTTAACGCGGAGAAACAATTCCTGGATGCGCT
GGGTATTACCACCGTTGCGGCGGTGCTGGGTGGCAGCATGGGTGGCGCG
CGTACCCTGGAGTGGGCGGCGATGTATCCGGAAATCGTTGGTGCGGCGG
CGGTGCTGGCGGTTAGCGCGCGTGCGAGCGCGTGGCAGATCGGCATTCA
GAGCGCGCAAATCAAGGCGATTGAAAACGATCACCACTGGCACGAGGGT
AACTACTATGAAAGCGGCTGCAACCCGGCGACCGGTCTGGGTGCGGCGC
GTCGTATTGCGCACCTGACCTACCGTGGTGAGCTGGAAATCGACGAGCG
TTTTGGCACCAAGGCGCAGAAAAACGAAAACCCGCTGGGTCCGTATCGT
AAGCCGGATCAACGTTTCGCGGTTGAGAGCTACCTGGACTATCAGGCGG
ATAAACTGGTTCAACGTTTTGACGCGGGTAGCTACGTGCTGCTGACCGA
TGCGCTGAACCGTCACGACATTGGCCGTGATCGTGGTGGCCTGAACAAG
GCGCTGGAGAGCATTAAAGTGCCGGTTCTGGTGGCGGGCGTTGACACCG
ATATCCTGTACCTGTATCACCAGCAAGAACACCTGAGCCGTAACCTGGG
TAACCTGCTGGCGATGGCGAAAATCGTTAGCCCGGTGGGTCATGATGCG
TTCCTGACCGAAAGCCGTCAAATGGATCGTATTGTGCGTAACTTCTTTA
GCCTGATCAGCCCGGACGAGAACAACCCGAGCACCTACATTGAATTTTA
TATCTAA.

In some embodiments, the bacterial cell comprises an exogenous nucleic acid molecule, the nucleotide comprising sequence:

(SEQ ID NO: 18)
ATGCGTATCCAGCGTTACATTCTGATGAGCGAGATCGCGCTGGAAGCGT
GGGGTGAGCATGAAGCGCTGCTGCTGAAGCCGCCGCGTAGCCCGCTGAG
CATCCCGCCGCCGAAGCCGCGTACCGCGGTGCTGTTCCCGCGTCGTGAG
GGTTTTTACACCGAGCTGGGTGGTTACCTGCCGGAAGTGCGTCTGCGTT
TCGAAACCTACGGCACCCTGAGCCGTCGTCGTGATAACGCGGTGCTGGT
TTTTCATGCGCTGACCGGTAGCGCGCACCTGGCGGGCACCTATGATGAG
GAAACCTTCCGTAGCCTGAGCCCGCTGGAGCAGGCGTTTGGTCGTGAAG
GTTGGTGGGATAGCCTGGTGGGTCCGGGTCGTATCCTGGACCCGGCGCT
GTACTATGTGGTTAGCGCGAACCACCTGGGTAGCTGCTATGGTAGCACC
GGTCCGCTGAGCCTGGACCCGCACACCGGTCGTCCGTATGGTCGTGATT
TCCCGCCGCTGACCATTCGTGATCTGGCGCGTGCGCAAGCGCGTCTGCT
GGACCACCTGGGTGTGGAGAAGGCGATCGTTATTGGTGGCAGCCTGGGT
GGCATGGTGGCGCTGGAGTTCGCGCTGATGTACCCGGAACGTGTTAAGA
AACTGGTGGTTCTGGCGGCGCCGGCGCGTCACGGCCCGTGGGCGCGTGC
GTTTAACCACCTGAGCCGTCAGGCGATCCTGCAAGATCCGGAATACCAG
AAGGGTAACCCGGCGCCGAAGGGTATGGCGCTGGCGCGTGGTATTGCGA
TGATGAGCTATCGTGCGCCGGAAGGTTTTGAAGCGCGTTGGGGTGCGGA
GCCGGAACTGGGCGAGACCTACCTGGACTATCAAGGTGAAAAGTTCCTG
CGTCGTTTTCACGCGGAGAGCTATCTGGTGCTGAGCCGTGCGATGGACA
CCCATGATGTGGGTCGTGGTCGTGGTGGCGTTGAGGAAGCGCTGAAACG
TCTGCGTGCGATCCCGAGCCTGTTTGTGGGCATTGACACCGATCTGCTG
TACCCGGCGTGGGAAGTTCGTCAGGCGGCGAAGGCGGCGGGTGCGCGTT
ATCGTGAGATCAAAAGCCCGCACGGCCATGATGCGTTCCTGATCGAAAC
CGATCAAGTTGAGGAAATTCTGGACGCGTTTCTGCCGTAA.

In some embodiments, metX comprises metX of LI, as disclosed herein, and comprises a nucleic acid sequence as set forth in SEQ ID NO: 13.

In some embodiments, metX comprises metX of SA, as disclosed herein, and comprises a nucleic acid sequence as set forth in SEQ ID NO: 14.

In some embodiments, metX comprises metX of DG, as disclosed herein, and comprises a nucleic acid sequence as set forth in SEQ ID NO: 15.

In some embodiments, metX comprises metX of CM, as disclosed herein, and comprises a nucleic acid sequence as set forth in SEQ ID NO: 16.

In some embodiments, metX comprises metX of GC, as disclosed herein, and comprises a nucleic acid sequence as set forth in SEQ ID NO: 17.

In some embodiments, metX comprises metX of TT, as disclosed herein, and comprises a nucleic acid sequence as set forth in SEQ ID NO: 18.

In some embodiments, a nucleic acid sequence as set forth in SEQ ID Nos: 13-18 comprises a codon optimized nucleic acid sequence, such as, but not limited to expression of metX as disclosed herein.

In some embodiments, codon optimized is for optimized expression, e.g., transcription, translation, or both, of a protein product of metX, in a cell.

In some embodiments, a bacterial cell comprises an exogenous nucleic acid molecule encoding metX. In some embodiments, a bacterial cell comprises an exogenous nucleic acid molecule comprising a nucleotide sequence set forth in SEQ ID Nos: 15 or 16.

In some embodiments, the bacterial cell comprises an exogenous nucleic acid molecule, of metY or metY optimized codon.

In some embodiments, the bacterial cell comprises an exogenous nucleic acid molecule, comprising the nucleotide sequence:

(SEQ ID NO: 19)
ATGGGTCCGCGTAACTATAAACCGGAGACCATTGCGCTGCACGGTGGCC
AGAGCCCGGACCCGAGCACCCTGAGCCGTGCGGTGCCGATTTACCAAAC
CACCAGCTATGTTTTCAAGAACACCGAGCACGCGGCGAAACTGTTTGGT
CTGCAAGAGTTCGGCAACATCTACACCCGTATTATGAACCCGACCACCG
ATGTTCTGGAGCAACGTATCGCGGCGCTGGAAGGTGGCGTGGCGGCGCT
GGCGACCGCGAGCGGTCAGGCGGCGGAAACCCTGGCGCTGCTGAACATC
GTGGAGGCGGGCCAAGAAATTGTTGCGAGCAGCAGCCTGTACGGTGGCA
CCTATAACCTGCTGCACTATACCTTTCCGAAGCTGGGTATCAAAGTGCA
CTTCGTTGACCCGAGCGATCCGGAGAACTTTCGTAAGGCGGTTAACGAC
AAAACCCGTGCGTTTTACGCGGAAACCCTGGGCAACCCGAAGCTGGATA
CCCTGAACCTGGAGGCGATTGCGAAAGTGGCGCACGACAGCGAAGTTCC
GCTGATCATTGATAACACCCTGCCGAGCCCGTACCTGGTTAACCCGATC
GAGCACGGTGCGGACATTGTGGTTCACAGCCTGACCAAGTTCCTGGGTG
GTCACGGCACCAGCATCGGTGGCATCATTGTGGACAGCGGCAAATTTAA
CTGGGGTAACGGCAAGTTCAAAAACTTTACCGAACCGGACCCGAGCTAT
CACGGTCTGAAGTTCTGGGAAGTGTTCGGCAAATTTGAACCGTTCGGTG
GCGTTAACATCGCGTACATCATTAAGGCGAAAGTGCAGGGTCTGCGTGA
TATGGGCGCGAGCATCAGCCCGTTTAACGCGTGGCAGATTCTGCAAGGT
GTTGAGACCCTGCCGCTGCGTATGCGTAAACACAGCGAGAACGCGCTGG
CGGTGGCGGAATATCTGACCAAGCACACCAAAGTGAGCTGGGTTAACTA
CCCGGGTCTGAAGATGGACAAAAACTACAGCCTGGCGAAGAAATATCAC
AAGAAAGATCTGTACGGCGCGATCCTGGGTTTCGGCATTAAGGGTGGCG
CGGTGGAGGCGAAGAAATTTATCGACGGTCTGGAACTGTTCAGCCTGCT
GGCGAACGTGGGCGATGCGAAAAGCCTGGTTATTCACCCGGCGAGCACC
ACCCACCAGCAACTGACCCCGGAGGAACAACTGAGCGCGGGTGTTACCC
CGGACTTCGTGCGTCTGAGCGTTGGCCTGGAGAACATCGAAGACATTCT
GTTTGATCTGGAGGAAGCGCTGAAGAAAGTGTAA.

In some embodiments, the bacterial cell comprises an exogenous nucleic acid molecule, comprising

(SEQ ID NO: 20)
ATGGACCTGCAAACCCTGGCGCTGCATGCGGGTTACGAAAAGGATAGCC
AGCGTACGATGGCGGTGCCGATCTACCAAACCACCGCGTATGAGTTCCG
TGACGTTGAACACGCGGCGAACCTGTTTGCGCTGAAGGAACTGGGCAAC
ATTTATACCCGTCTGAACAACCCGACCACCGATGTGTTCGAGAAACGTT
TTACCGCGCTGGAGGGTGGCGAAGCGGCGATTGCGACCGCGAGCGGTAT
GAGCGCGATTTTCTTTGCGCTGGCGAACGCGGCGCAGGCGGGTGACAAC
ATCATTTGCGCGAACCAACTGTACGGTGGCAGCCTGACCCTGAGCACCC
ACACCCTGAAGCGTTTCGGTATCGAAGCGCGTTTCTTTGACGTTCACAA
ACCGCAGCAAATCGAGGTGCTGATCGATGAAAAGACCAAGGTTATCTTC
TTTGAAAGCCTGACCAACCCGAGCATCGACGTGGCGGATATTGAGGCGC
TGACCGCGATTGCGAACAAACACAACATCCTGACCATTGTGGACAACAC
CGTTGCGACCCCGGTGCTGTGCCGTCCGTTTGAGTTTGGTGCGGATATC
ACCGTTCACAGCGCGAGCAAGTATACCACCGGTCAGGGCCTGGCGATCG
GTGGCATTCTGGTTGAACGTAAAGGCCTGGTGGACAAGCTGAAAAACAA
CCCGCGTTACAGCCACTTCAACGAGCCGGACGCGAGCTACCACGGTCTG
GTTTATACCGATACCGGTCTGCCGCCGTATACCCTGCGTGCGCGTCTGA
GCCTGCTGCGTGATCTGGGTGCGGTGGTTAGCCCGTTCAACAGCTGGCT
GTTTATCCAAGGCATTGAAACCCTGAGCCTGCGTATGAAGGAGCACAGC
AAAAACGCGCTGATGCTGGCGGAGTTCCTGGAAAGCCACAAGAAAGTGA
AGAAAGTTAACTACCCGGGTCTGAAGAGCAACGCGAACTACAAGAACGC
GCAGAAATATTTTCAAGATGGTCTGTGCAGCGGCCTGCTGAGCTTCGAG
GTGGAAGACTTTGATGAAGCGACCAAGATCGTTGACGCGACCAAACTGT
ATAGCCTGGTGGTTAACATTGGTGATAGCAAAAGCATCATTACCCATCC
GGCGAGCACCACCCACCAGCAACTGAGCCACGAGGAACTGATTGCGTGC
GGTGTGCCGGAGGGTCTGATCCGTATTAGCTGCGGCCTGGAGAGCGTTA
AGGACCTGATTGAAGATCTGAAACAAGCGCTGGAGGCGTAA.

In some embodiments, the bacterial cell comprises an exogenous nucleic acid molecule, comprising

(SEQ ID NO: 21)
ATGGCGAGCAACACCCTGCACTTCGAGACCCTGCAAGTGCACGCGGGTC
AACATCCGGACCCGGCGACCGGCGCGCAAGCGGTGCCGATTTACGCGAC
CAACGCGTATGTTTTTGAAAGCCCGGAACATGCGGCGGACCTGTTCGGT
CTGCGTGCGTTTGGCAACATTTACAGCCGTATCATGAACCCGACCAACG
CGGTTCTGGAGGAACGTATTGCGGCGCTGGAAGGTGGCGTGGGTGCGCT
GGCGGTTGCGAGCGGTCATGCGGCGCAGTTCCTGGCGATTACCACCGTG
GCGCAAGCGGGTGATAACATCGTTAGCACCCCGAACCTGTACGGTGGCA
CCGTGAACCAATTTCGTGTTACCCTGCGTCGTCTGGGCATTGAAGTGCG
TTTCACCAGCAAGGACGAACGTCCGGAGGAATTTGCGGCGCTGATCGAC
GATCGTACCCGTGCGGTTTATCTGGAGACCCTGGGTAACCCGGCGCTGA
ACGTGCCGGACTTTGAGGGTATTGCGGAAGTTGCGCATGCGCGTGGCGT
GGCGGTTTTTGTGGATAACACCTTTGGTGCGGGTGGCTACTATTGCCAA
CCGCTGCGTCACGGCGCGGATGTGGTTCTGCACAGCGCGAGCAAATGGA
TTGGTGGCCACGGTAACGGCATCGGTGGTCTGCTGGTTGACGGTGGCAC
CTTTGATTGGGGTAACGGCCGTTACCCGCTGCTGACCGAGCCGAGCCCG
AGCTATCACGGTCTGAGCTTCTGGGAGGCGTTTGGTGAGGGTAACGCGC
TGGGTCTGCCGAACATTGCGTTCATTACCCGTGCGCGTACCGAAGGTCT
GCGTGATCTGGGTCCGACCCTGGCGCCGCAGCAAGCGTGGCAGTTTCTG
CAAGGTGTGGAGACCCTGAGCCTGCGTGCGGAACGTCATGCGCAGAACG
CGCTGGCGCTGGCGAGCTGGCTGAGCGGTCACCCGGATGTGAGCCGTGT
TACCTATCCGGGCCTGAGCAACCACCCGCACTACGATCGTGCGCAAACC
TATCTGCCGCGTGGTGCGGGTGCGGTTCTGACCTTTGAGCTGCGTGGTG
GCCGTGCGGCGGGTGAAGCGTTTATTGGTGCGGTGCGTCTGGCGCAGCA
TGTGGCGAACGTTGGTGACACCCGTACCCTGGTTATTCATCCGGCGAGC
ACCACCCACAGCCAGCTGGATGAAGCGGCGCAAGCGGCGGCGGGTGTGA
CCCCGGGCCTGGTTCGTGTGAGCGTTGGTATCGAGCACATTGACGATAT
CCGTGAAGATTTTGCGCAGGCGCTGGCGACCGCGCTGGTTGATGCGGAG
GGTGCGTAA.

In some embodiments, the bacterial cell comprises an exogenous nucleic acid molecule, comprising

(SEQ ID NO: 22)
ATGGGTAGCAAGAACTACCGTTTCGAGACCCTGCAAGTGCACGGTGGCC
AAGAAGTTGACCCGACCACCAACAGCCGTGCGGTGCCGATCTACCAGAC
CAGCAGCTATGTTTTTAACAGCGCGGAGCATGGTGCGAACCTGTTCGCG
CTGAAGGAATTTGGCAACATCTATACCCGTATTATGAACCCGACCAGCG
ACGTTTTCGAGAAACGTATGGCGGCGCTGGAAGGTGGCGTGGCGGCGGT
TGCGACCGCGAGCGGTCAGGCGGCGCAATTCCTGGCGCTGAACAACTTT
CTGAGCGTGGGCGATAACTTCGTTACCAGCCCGTTTCTGTACGGTGGCA
GCTATAACCAATTCAAAGTGAGCTTTAAACGTATCGGTATTGAGGCGCG
TTTTGCGAAGAGCGACAAAGTTGACGATCTGGCGGCGGAAATCAACGAT
AAGACCAAAGCGATCTACGTGGAGACCATTGGCAACCCGGAGTTCAACG
TTCCGGACTTTGAAGCGATCGCGGCGCTGGCGAAGAAACACGACATTCC
GCTGGTTGTGGATAACACCTTCGGTGCGGGTGGCTATCTGTGCCAGCCG
ATCAAGCACGGCGCGAACATTGTGACCAGCAGCGCGACCAAATGGATCG
GTGGTCACGGCACCAGCATTGGTGGCATCATTGTTGATGGTGGCAACTA
CAACTGGGGTAACGGCAAGTTCCCGCAATTTAGCGAGCCGAGCGAAGGT
TATCACGGCCTGAACTTCTGGGAGACCTTTGGTGACAACAACCCGCTGG
GTCTGCCGAACATTGCGTTCGCGATTCGTGCGCGTGTGGAAGGTCTGCG
TGATTTTGGCCCGGCGATCAGCCCGTTCAACAGCTTTCTGCTGCTGCAA
GGTCTGGAGACCCTGAGCCTGCGTGTGCAACGTACCGTTGACAACGCGC
TGGAGCTGGCGAAATGGCTGGAAGCGCACCCGAAGGTGAAAAGCGTTAA
CTATCCGGGTCTGACCAACAGCCCGTACCACGCGACCGCGAAGAAATAT
CTGACCCACGGTTTCGGTGGCGTGCTGAGCTTTGAGATTGAAGGCGATA
AGGAGACCGCGAGCAACTTTATCAACAACCTGGAACTGATTAGCCACCT
GGCGAACGTTGGTGACGCGAAAACCCTGATCATTCAGCCGAGCGCGACC
ACCCACCAGCAACTGAGCGATGAAGCGCAAATTGCGGCGGGTGTGACCC
CGAGCCTGCTGCGTATTAGCAGCGGCATCGAGCACATTGAAGACCTGAA
GGCGGATCTGACCGCGGCGTTCGATAAAATCTAA.

In some embodiments, the bacterial cell comprises an exogenous nucleic acid molecule, comprising the nucleotide sequence:

(SEQ ID NO: 23)
ATGGGTCCGAAGTACGACAACAGCAACGCGGATCAGTGGGGCTTCGAGA
CCCGTAGCATCCACGCGGGTCAGAGCGTTGACGCGCAAACCAGCGCGCG
TAACCTGCCGATTTACCAGAGCACCGCGTTCGTGTTTGACAGCGCGGAG
CACGCGAAACAACGTTTCGCGCTGGAAGATCTGGGCCCGGTTTATAGCC
GTCTGACCAACCCGACCGTGGAGGCGCTGGAAAACCGTATTGCGAGCCT
GGAGGGTGGCGTTCATGCGGTGGCGTTTAGCAGCGGTCAGGCGGCGACC
ACCAACGCGATCCTGAACCTGGCGGGTGCGGGTGACCACATTGTTACCA
GCCCGCGTCTGTATGGTGGCACCGAAACCCTGTTCCTGATCACCCTGAA
CCGTCTGGGCATTGATGTTAGCTTTGTGGAGAACCCGGATGATCCGGAA
AGCTGGCAGGCGGCGGTTCAACCGAACACCAAGGCGTTCTTTGGCGAGA
CCTTTGCGAACCCGCAAGCGGACGTGCTGGATATCCCGGCGGTTGCGGA
AGTGGCGCACCGTAACAGCGTTCCGCTGATCATTGACAACACCATTGCG
ACCGCGGCGCTGGTGCGTCCGCTGGAGCTGGGTGCGGATGTGGTTGTGG
CGAGCCTGACCAAGTTCTACACCGGTAACGGCAGCGGTCTGGGTGGCGT
TCTGATCGACGGTGGCAAATTTGATTGGACCGTGGAAAAGGACGGCAAA
AGCGTTTTCCCGTATTTTGTTACCCCGGATGCGGCGTACCACGGTCTGA
AGTATGCGGATCTGGGTGCGCCGGCGTTTGGTCTGAAAGTTCGTGTGGG
CCTGCTGCGTGACACCGGTAGCACCCTGAGCGCGTTTAACGCGTGGGCG
GCGGTTCAAGGCATCGATACCCTGAGCCTGCGTCTGGAGCGTCACAACG
AAAACGCGATTAAGGTGGCGGAGTTCCTGAACAACCACGAGAAGGTTGA
AAAAGTGAACTTTGCGGGTCTGAAGGATAGCCCGTGGTACGCGACCAAG
GAAAAACTGGGCCTGAAATATACCGGTAGCGTGCTGACCTTCGAGATCA
AGGGTGGCAAAGACGAAGCGTGGGCGTTTATTGATGCGCTGAAACTGCA
CAGCAACCTGGCGAACATCGGCGACGTTCGTAGCCTGGTTGTGCATCCG
GCGACCACCACCCATAGCCAAAGCGATGAGGCGGGCCTGGCGCGTGCGG
GTGTGACCCAAAGCACCGTTCGTCTGAGCGTGGGTATCGAGACCATTGA
CGATATCATTGCGGACCTGGAAGGTGGCTTCGCGGCGATTTAA.

In some embodiments, the bacterial cell comprises an exogenous nucleic acid molecule, comprising the nucleotide sequence:

(SEQ ID NO: 24)
ATGGGTCGTTTCGAGACCCTGCAACTGCATGCGGGTTATGAGCCGGAAC
CGACCACCCTGAGCCGTCAAGTTCCGATCTACCCGACCACCAGCTATGT
GTTTAAAAGCCCGGAGCATGCGGCGAACCTGTTCGCGCTGAAGGAATTT
GGTAACATCTATAGCCGTATTATGAACCCGACCGTGGACGTTCTGGAGA
AACGTCTGGCGGCGCTGGAAGGTGGCAAAGCGGCGCTGGCGACCGCGAG
CGGTCATGCGGCGCAGTTTCTGGCGCTGACCACCCTGGCGCAAGCGGGC
GATAACATCGTTAGCACCCCGAACCTGTACGGTGGCACCTTCAACCAGT
TTAAGGTTACCCTGAAACGTCTGGGCATTGAAGTGCGTTTCACCAGCCG
TGAGGAACGTCCGGAGGAATTTCTGGCGCTGACCGATGAGCGTACCCGT
GCGTGGTGGGTGGAAAGCATCGGTAACCCGGCGCTGAACATTCCGGACC
TGGAGGCGCTGGCGCAAGCGGCGCGTGAAAAAGGTGTGGCGCTGATCGT
TGATAACACCTTCGGCATGGGTGGCTATCTGCTGCGTCCGCTGGCGTGG
GGCGCGGCGCTGGTTACCCACAGCCTGACCAAGTGGGTGGGTGGCCACG
GTGCGGTTATTGCGGGTGCGATTGTGGATGGTGGCAGCTTTCCGTGGGA
GGGTGGCCGTTACCCGCTGCTGACCGAACCGCAGCCGGGTTATCACGGC
CTGCGTCTGACCGAGGCGTTCGGTGAACTGGCGTTTATTGTGAAAGCGC
GTGTTGACGGCCTGCGTGATCAAGGTCAAGCGCTGGGTCCGTTTGAGGC
GTGGGTGGTTCTGCTGGGTATGGAAACCCTGAGCCTGCGTGCGGAGCGT
CATGTGGAAAACACCCTGCATCTGGCGCACTGGCTGCTGGAGCAGCCGC
AAGTGGCGTGGGTTAACTATCCGGGTCTGCCGCACCACCCGCACCACGA
CCGTGCGCAGAAGTATTTCAAGGGTAAACCGGGCGCGGTTCTGACCTTT
GGCCTGAAGGGTGGCTACGAAGCGGCGAAACGTTTTATCAGCCGTCTGA
AGCTGATTAGCCACCTGGCGAACGTGGGTGACACCCGTACCCTGGCGAT
TCACCCGGCGAGCACCACCCACAGCCAACTGAGCCCGGAGGAACAGGCG
CAAGCGGGTGTTAGCCCGGAGATGGTGCGTCTGAGCGTTGGCCTGGAGC
ACGTGGAAGATCTGAAGGCGGAGCTGAAAGAAGCGCTGGCGTAA.

In some embodiments, metY comprises metY of LI, as disclosed herein, and comprises a nucleic acid sequence as set forth in SEQ ID NO: 19.

In some embodiments, metY comprises metY of SA, as disclosed herein, and comprises a nucleic acid sequence as set forth in SEQ ID NO: 20.

In some embodiments, metY comprises metY of DG, as disclosed herein, and comprises a nucleic acid sequence as set forth in SEQ ID NO: 21.

In some embodiments, metY comprises metY of CM, as disclosed herein, and comprises a nucleic acid sequence as set forth in SEQ ID NO: 22.

In some embodiments, metY comprises metY of GC, as disclosed herein, and comprises a nucleic acid sequence as set forth in SEQ ID NO: 23.

In some embodiments, metY comprises metY of TT, as disclosed herein, and comprises a nucleic acid sequence as set forth in SEQ ID NO: 24.

In some embodiments, a nucleic acid sequence as set forth in SEQ ID Nos: 19-24 comprises a codon optimized nucleic acid sequence, such as, but not limited to expression of metY as disclosed herein.

In some embodiments, a bacterial cell comprises an exogenous nucleic acid molecule encoding metY. In some embodiments, a bacterial cell comprises an exogenous nucleic acid molecule comprising a nucleotide sequence set forth in SEQ ID Nos: 21 or 22.

In some embodiments, the nucleic acid or plurality thereof disclosed herein comprises a synonymous mutation (e.g., not affecting the polypeptide sequence) for any one of SEQ ID NOs: 13-24. In some embodiments, the nucleic acid or plurality thereof comprises a nonsynonymous mutation for any one of SEQ ID NOs: 13-24, however in this case, the nucleic acid comprising the nonsynonymous mutation would still be translated to a functional analog, as disclosed herein.

In some embodiments, the nucleic acid molecule encoding HAT (metX) and the nucleic acid molecule encoding OHS (metY) are operably linked. The term “operably linked” is intended to mean that the nucleotide sequence encoding HAT (metX) and the nucleotide sequence encoding OHS (metY) are present in the same nucleotide molecule and are both linked to a regulatory element or elements in a manner that allows for expression of their nucleotide sequence (e.g., in an in vitro transcription/translation system or in a host cell when the vector is introduced into the host cell).

In some embodiments, metX encoding or encoded sequence or metY encoding or encoded sequence being derived from the same microorganism. In some embodiments, the operably linked nucleic acid molecule disclosed herein comprises at least one of: (a) SEQ ID NO: 19 and SEQ ID NO: 13, (b) SEQ ID NO: 20 and SEQ ID NO: 14, (c) SEQ ID NO: 21 and SEQ ID NO: 15, (d) SEQ ID NO: 22 and SEQ ID NO: 16, (e) SEQ ID NO: 23 and SEQ ID NO: 17; and (f) SEQ ID NO: 24, and SEQ ID NO: 18.

In some embodiments, a bacterial cell comprises a nucleic acid molecule or a plurality thereof, encoding SEQ ID NO: 19, SEQ ID NO: 13, or both. In some embodiments, the bacterial cell comprises a nucleic acid molecule or a plurality thereof, encoding SEQ ID NO: 20 and SEQ ID NO: 14. In some embodiments, the bacterial cell comprises a nucleic acid or plurality thereof, encoding SEQ ID NO: 21 and SEQ ID NO: 15. In some embodiments, the bacterial cell comprises a nucleic acid or plurality thereof, encoding SEQ ID NO: 22 and SEQ ID NO: 16. In some embodiments, the bacterial cell comprises a nucleic acid or plurality thereof, encoding SEQ ID NO: 23 and SEQ ID NO: 17. In some embodiments, the bacterial cell comprises a nucleic acid or plurality thereof, encoding SEQ ID NO: 24 and SEQ ID NO: 18.

In some embodiments, the nucleic acid molecule comprises or is operably linked to a promoter. In some embodiments, the promoter comprises a single promoter. In some embodiments, the promoter comprises or consists of 35-50 nucleotides. In some embodiments, the promoter comprises the nucleotide sequence:

(SEQ ID NO: 25)
TTTATTCTTGACACTAGTCGGCCAAAATGATATAATACCTGA.

In some embodiments, the nucleotide sequence of the promoter is positioned or located upstream to the nucleotide sequence of metY. In some embodiments, the nucleotide sequence of metY is positioned or located between the nucleotide sequence of the promotor and the nucleotide sequence of metX. In some embodiments, any one of SEQ ID NOs: 19-24, is positioned or located between SEQ ID NO: 25, and any one of SEQ ID NOs: 13-18.

In some embodiments, nucleic acid molecule further comprises a terminator sequence.

As used herein, the term “terminator sequence” refers to a sequence in that signals, dictates, promotes, induces, required, or any combination thereof, for termination of transcription. In some embodiments, the terminator sequence comprises 25-35 nucleotides. In some embodiments, the terminator sequence comprises the nucleotide sequence: CAACCTGGAGGCGGGCGCAGGCCCGCCTTTT (SEQ ID NO: 26) or a functional analog thereof having at 80-100% sequence identity or homology thereto.

In some embodiments, the terminator sequence is located or position downstream to the nucleotide sequence of metX. In some embodiments, the nucleotide sequence of metX is positioned or located between the nucleotide sequence of metY and the nucleotide sequence of the terminator. In some embodiments, any one of SEQ ID NOs: 13-18, is positioned or located between any one of SEQ ID NOs: 19-24, and SEQ ID NO: 26.

In some embodiments, the nucleic acid molecule further comprises a ribosome binding site (RBS).

As used herein, the term “ribosome binding site” or “RBS” refers to a nucleotide sequence located or positioned upstream of a start codon of or in a messenger RNA (mRNA) transcript that is responsible for the recruitment of a ribosome during the initiation of translation. In some embodiments, the nucleic acid molecule further comprises a RBS sequence located or positioned upstream to at least one of the sequences comprising metX and metY. In some embodiments, the nucleic acid molecule comprises RBS sequence located or positioned between the promotor and sequence comprising metY. In some embodiments, the nucleic acid molecule comprises RBS sequence located or positioned between the sequence comprising metY and the sequence comprising metX. In some embodiments, the nucleic acid molecule comprises RBS sequence located or positioned between the promotor and sequence comprising metY and RBS sequence between the sequence comprising metY and the sequence comprising metX. In some embodiments the RBS sequence consists of or comprises 10-15 nucleotides. In some embodiments the RBS sequence comprises the sequence: AGAGGTATATATTA (SEQ ID NO: 27).

In some embodiments, the nucleic acid molecule comprises SEQ ID NO: 27, located or positioned between SEQ ID NO: 25 and any one of SEQ ID NOs: 19-24. In some embodiments, the nucleic acid molecule comprises SEQ ID NO: 27, located or positioned between any one of SEQ ID NOs: 19-24, and any one of SEQ ID NOs: 13-18. In some embodiments, the nucleic acid molecule comprises SEQ ID NO: 27, located or positioned between SEQ ID NO: 25 and any one of SEQ ID NOs: 19-24, and further comprises SEQ ID NO: 27, located or positioned between any one of SEQ ID NOs: 19-24, and any one of SEQ ID NOs: 13-18.

In some embodiments, the nucleic acid comprises 25-35 nucleotides between the promotor sequence and the sequence encoding metY. In some embodiments, the nucleic acid comprises 15-40 nucleotides between the promotor sequence and the sequence encoding metY. In some embodiments, the sequence located or positioned between SEQ ID NO: 25, and any one of SEQ ID NOs: 19-24, comprises or consists of the sequence:

(SEQ ID NO: 34)
GTTTAACTTTAAGAGAGGTATATATTACC.

In some embodiments, the nucleic acid molecule comprises 15-25 nucleotides located or positioned between the sequence encoding metY and the sequence encoding metX. In some embodiments, the nucleic acid comprises 15-35 nucleotides located or positioned between the sequence encoding metY and the sequence encoding metX. In some embodiments, the sequence located or positioned between any one of SEQ ID NOs: 19-24, and any one of SEQ ID NOs: 13-18, comprises or consists of the sequence: GGATCCAGAGGTATATATTA (SEQ ID NO: 35).

In some embodiments, the bacterial cell comprises an exogenous nucleic acid molecule or a plurality thereof, comprising the sequence:

(SEQ ID NO: 28)
TTTATTCTTGACACTAGTCGGCCAAAATGATATAATACCTGAGTTTAAC
TTTAAGAGAGGTATATATTACCATGGGTCCGCGTAACTATAAACCGGAG
ACCATTGCGCTGCACGGTGGCCAGAGCCCGGACCCGAGCACCCTGAGCC
GTGCGGTGCCGATTTACCAAACCACCAGCTATGTTTTCAAGAACACCGA
GCACGCGGCGAAACTGTTTGGTCTGCAAGAGTTCGGCAACATCTACACC
CGTATTATGAACCCGACCACCGATGTTCTGGAGCAACGTATCGCGGCGC
TGGAAGGTGGCGTGGCGGCGCTGGCGACCGCGAGCGGTCAGGCGGCGGA
AACCCTGGCGCTGCTGAACATCGTGGAGGCGGGCCAAGAAATTGTTGCG
AGCAGCAGCCTGTACGGTGGCACCTATAACCTGCTGCACTATACCTTTC
CGAAGCTGGGTATCAAAGTGCACTTCGTTGACCCGAGCGATCCGGAGAA
CTTTCGTAAGGCGGTTAACGACAAAACCCGTGCGTTTTACGCGGAAACC
CTGGGCAACCCGAAGCTGGATACCCTGAACCTGGAGGCGATTGCGAAAG
TGGCGCACGACAGCGAAGTTCCGCTGATCATTGATAACACCCTGCCGAG
CCCGTACCTGGTTAACCCGATCGAGCACGGTGCGGACATTGTGGTTCAC
AGCCTGACCAAGTTCCTGGGTGGTCACGGCACCAGCATCGGTGGCATCA
TTGTGGACAGCGGCAAATTTAACTGGGGTAACGGCAAGTTCAAAAACTT
TACCGAACCGGACCCGAGCTATCACGGTCTGAAGTTCTGGGAAGTGTTC
GGCAAATTTGAACCGTTCGGTGGCGTTAACATCGCGTACATCATTAAGG
CGAAAGTGCAGGGTCTGCGTGATATGGGCGCGAGCATCAGCCCGTTTAA
CGCGTGGCAGATTCTGCAAGGTGTTGAGACCCTGCCGCTGCGTATGCGT
AAACACAGCGAGAACGCGCTGGCGGTGGCGGAATATCTGACCAAGCACA
CCAAAGTGAGCTGGGTTAACTACCCGGGTCTGAAGATGGACAAAAACTA
CAGCCTGGCGAAGAAATATCACAAGAAAGATCTGTACGGCGCGATCCTG
GGTTTCGGCATTAAGGGTGGCGCGGTGGAGGCGAAGAAATTTATCGACG
GTCTGGAACTGTTCAGCCTGCTGGCGAACGTGGGCGATGCGAAAAGCCT
GGTTATTCACCCGGCGAGCACCACCCACCAGCAACTGACCCCGGAGGAA
CAACTGAGCGCGGGTGTTACCCCGGACTTCGTGCGTCTGAGCGTTGGCC
TGGAGAACATCGAAGACATTCTGTTTGATCTGGAGGAAGCGCTGAAGAA
AGTGTAAGGATCCAGAGGTATATATTAATGAACGAGACCGGTAGCATCG
GCATCATTGAAACCAAGTACGCGGAGTTCAAAGAACTGATCCTGAACAA
CGGTAGCGTGCTGAGCCCGGTGGTTATTGCGTACGAGACCTATGGCACC
CTGAGCAGCAGCAAGAACAACGCGATCCTGATTTGCCATGCGCTGAGCG
GTGATGCGCATGCGGCGGGTTATCACAGCGGCAGCGATAAGAAACCGGG
TTGGTGGGACGATTACATTGGTCCGGGCAAGAGCTTCGACACCAACCAG
TATTTTATCATTTGCAGCAACGTTATCGGTGGCTGCAAAGGTAGCAGCG
GCCCGCTGAGCATTCATCCGGAGACCAGCACCCCGTATGGTAGCCGTTT
CCCGTTTGTGAGCATCCAGGATATGGTTAAGGCGCAAAAGCTGCTGGTT
GAGAGCCTGGGTATTGAAAAACTGTTCTGCGTTGCGGGTGGCAGCATGG
GTGGCATGCAAGCGCTGGAATGGAGCATCGCGTATCCGAACAGCCTGAG
CAACTGCATTGTTATGGCGAGCACCGCGGAGCACAGCGCGATGCAGATC
GCGTTTAACGAAGTTGGTCGTCAAGCGATTCTGAGCGACCCGAACTGGA
AGAACGGCCTGTACGATGAGAACAGCCCGCGTAAAGGTCTGGCGCTGGC
GCGTATGGTGGGTCACATCACCTATCTGAGCGACGATAAGATGCGTGAA
AAATTCGGTCGTAACCCGCCGCGTGGCAACATCCTGAGCACCGACTTTG
CGGTTGGTAGCTACCTGATTTATCAGGGCGAGAGCTTCGTGGACCGTTT
TGATGCGAACAGCTACATCTATGTTACCAAGGCGCTGGACCACTACAGC
CTGGGCAAGGGCAAAGAACTGACCGCGGCGCTGAGCAACGCGACCTGCC
GTTTCCTGGTGGTTAGCTACAGCAGCGATTGGCTGTATCCGCCGGCGCA
AAGCCGTGAGATTGTGAAGAGCCTGGAAGCGGCGGACAAACGTGTGTTC
TACGTTGAGCTGCAAAGCGGTGAAGGCCACGATAGCTTTCTGCTGAAGA
ACCCGAAACAAATCGAGATTCTGAAAGGTTTTCTGGAAAACCCGAACTA
ACTCGAGCAACCTGGAGGCGGGCGCAGGCCCGCCTTTT.

In some embodiments, the bacterial cell comprises an exogenous nucleic acid molecule or a plurality thereof, comprising the sequence:

(SEQ ID NO: 29)
TTTATTCTTGACACTAGTCGGCCAAAATGATATAATACCTGAGTTTAAC
TTTAAGAGAGGTATATATTACCATGGACCTGCAAACCCTGGCGCTGCAT
GCGGGTTACGAAAAGGATAGCCAGCGTACGATGGCGGTGCCGATCTACC
AAACCACCGCGTATGAGTTCCGTGACGTTGAACACGCGGCGAACCTGTT
TGCGCTGAAGGAACTGGGCAACATTTATACCCGTCTGAACAACCCGACC
ACCGATGTGTTCGAGAAACGTTTTACCGCGCTGGAGGGTGGCGAAGCGG
CGATTGCGACCGCGAGCGGTATGAGCGCGATTTTCTTTGCGCTGGCGAA
CGCGGCGCAGGCGGGTGACAACATCATTTGCGCGAACCAACTGTACGGT
GGCAGCCTGACCCTGAGCACCCACACCCTGAAGCGTTTCGGTATCGAAG
CGCGTTTCTTTGACGTTCACAAACCGCAGCAAATCGAGGTGCTGATCGA
TGAAAAGACCAAGGTTATCTTCTTTGAAAGCCTGACCAACCCGAGCATC
GACGTGGCGGATATTGAGGCGCTGACCGCGATTGCGAACAAACACAACA
TCCTGACCATTGTGGACAACACCGTTGCGACCCCGGTGCTGTGCCGTCC
GTTTGAGTTTGGTGCGGATATCACCGTTCACAGCGCGAGCAAGTATACC
ACCGGTCAGGGCCTGGCGATCGGTGGCATTCTGGTTGAACGTAAAGGCC
TGGTGGACAAGCTGAAAAACAACCCGCGTTACAGCCACTTCAACGAGCC
GGACGCGAGCTACCACGGTCTGGTTTATACCGATACCGGTCTGCCGCCG
TATACCCTGCGTGCGCGTCTGAGCCTGCTGCGTGATCTGGGTGCGGTGG
TTAGCCCGTTCAACAGCTGGCTGTTTATCCAAGGCATTGAAACCCTGAG
CCTGCGTATGAAGGAGCACAGCAAAAACGCGCTGATGCTGGCGGAGTTC
CTGGAAAGCCACAAGAAAGTGAAGAAAGTTAACTACCCGGGTCTGAAGA
GCAACGCGAACTACAAGAACGCGCAGAAATATTTTCAAGATGGTCTGTG
CAGCGGCCTGCTGAGCTTCGAGGTGGAAGACTTTGATGAAGCGACCAAG
ATCGTTGACGCGACCAAACTGTATAGCCTGGTGGTTAACATTGGTGATA
GCAAAAGCATCATTACCCATCCGGCGAGCACCACCCACCAGCAACTGAG
CCACGAGGAACTGATTGCGTGCGGTGTGCCGGAGGGTCTGATCCGTATT
AGCTGCGGCCTGGAGAGCGTTAAGGACCTGATTGAAGATCTGAAACAAG
CGCTGGAGGCGTAAGGATCCAGAGGTATATATTAATGAGCCTGAACCTG
CAAACCTATACCGAGCACTTCACCAACCCGCTGTACCTGGAGAGCGGTC
GTATCCTGGAACCGTACGATATTACCTACGAGACCTATGGCACCATGAA
CGAAGACAAGAGCAACGTGGTTGTGGTTTGCCATGCGCTGACCGGTAGC
CACCATGCGGCGGGTCTGTATGAGGATGAAACCAAACCGGGTTGGTGGG
ACGGCTTCATCGGTAGCGGCAAGGCGATTGACACCGATAAATACTTCGT
GATTTGCAGCAACGTTATTGGTAGCTGCTTTGGTAGCACCGGCCCGATG
AGCCTGCAACACCCGTATCAAGAACCGTACCGTTATAAGTTCCCGGTGG
TTAGCATCAAGGATATGGTGAAAGCGCAACGTATTCTGTTTGACCGTCT
GGATATCCACCGTGTGCACGCGATTGTTGGTGGCAGCATGGGTGGCATG
CAGGCGCTGCAATTCGCGATCCACTACCCGAACTTTGCGAACAAGATCA
TTGCGCTGGCGACCACCCATGCGACCCAGCCGTGGGCGATCGCGTTCAA
CAAAGTGGCGCAAGAGAGCATTCTGAACGACCCGGATTTTAAGCAGGGT
TACTATGACCCGGATCTGCTGAAAGAACAAGGTCTGAGCGGTATGGCGG
TTGGTCGTATGGCGGGTCACATCAGCTTCCTGAGCCACGAGAGCATGCG
TGAAAAGTTTGGTCGTGATTATAAACTGACCGACGGTCTGTACGAGCTG
TTCGGCAAGTTTCAGGTGGAGAGCTACCTGGAATATAACGGCTACAACT
TCACCAAGTGGTTTGATCCGCTGGCGTACCTGTACATCACCAAGGCGAT
CAACATCTACGATCTGAGCCGTGGTTTCGACAGCCTGGCGGAGGCGCTG
AAGCGTGTGACCAGCGCGCTGTACCTGGTTAGCTTTAAAAACGACCTGC
TGTTCAAGAACTTTGAGATGAAAGAAATCGCGGACGAACTGGATAAGAT
TGGTAACAAAAACCACAGCTATATCGACGTTAAAAGCGATTACGGCCAC
GACGCGTTCCTGGTGGAGCTGAACAAGTTTGAAAACCACGTTAAAGACG
CGCTGAACGGCTAACTCGAGCAACCTGGAGGCGGGCGCAGGCCCGCCTT
TT.

In some embodiments, the bacterial cell comprises an exogenous nucleic acid molecule or a plurality 1><CWU-Call number 86 thereof, comprising the sequence:

(SEQ ID NO: 30)
TTTATTCTTGACACTAGTCGGCCAAAATGATATAATACCTGAGTTTAAC
TTTAAGAGAGGTATATATTACCATGGCGAGCAACACCCTGCACTTCGAG
ACCCTGCAAGTGCACGCGGGTCAACATCCGGACCCGGCGACCGGCGCGC
AAGCGGTGCCGATTTACGCGACCAACGCGTATGTTTTTGAAAGCCCGGA
ACATGCGGCGGACCTGTTCGGTCTGCGTGCGTTTGGCAACATTTACAGC
CGTATCATGAACCCGACCAACGCGGTTCTGGAGGAACGTATTGCGGCGC
TGGAAGGTGGCGTGGGTGCGCTGGCGGTTGCGAGCGGTCATGCGGCGCA
GTTCCTGGCGATTACCACCGTGGCGCAAGCGGGTGATAACATCGTTAGC
ACCCCGAACCTGTACGGTGGCACCGTGAACCAATTTCGTGTTACCCTGC
GTCGTCTGGGCATTGAAGTGCGTTTCACCAGCAAGGACGAACGTCCGGA
GGAATTTGCGGCGCTGATCGACGATCGTACCCGTGCGGTTTATCTGGAG
ACCCTGGGTAACCCGGCGCTGAACGTGCCGGACTTTGAGGGTATTGCGG
AAGTTGCGCATGCGCGTGGCGTGGCGGTTTTTGTGGATAACACCTTTGG
TGCGGGTGGCTACTATTGCCAACCGCTGCGTCACGGCGCGGATGTGGTT
CTGCACAGCGCGAGCAAATGGATTGGTGGCCACGGTAACGGCATCGGTG
GTCTGCTGGTTGACGGTGGCACCTTTGATTGGGGTAACGGCCGTTACCC
GCTGCTGACCGAGCCGAGCCCGAGCTATCACGGTCTGAGCTTCTGGGAG
GCGTTTGGTGAGGGTAACGCGCTGGGTCTGCCGAACATTGCGTTCATTA
CCCGTGCGCGTACCGAAGGTCTGCGTGATCTGGGTCCGACCCTGGCGCC
GCAGCAAGCGTGGCAGTTTCTGCAAGGTGTGGAGACCCTGAGCCTGCGT
GCGGAACGTCATGCGCAGAACGCGCTGGCGCTGGCGAGCTGGCTGAGCG
GTCACCCGGATGTGAGCCGTGTTACCTATCCGGGCCTGAGCAACCACCC
GCACTACGATCGTGCGCAAACCTATCTGCCGCGTGGTGCGGGTGCGGTT
CTGACCTTTGAGCTGCGTGGTGGCCGTGCGGCGGGTGAAGCGTTTATTG
GTGCGGTGCGTCTGGCGCAGCATGTGGCGAACGTTGGTGACACCCGTAC
CCTGGTTATTCATCCGGCGAGCACCACCCACAGCCAGCTGGATGAAGCG
GCGCAAGCGGCGGCGGGTGTGACCCCGGGCCTGGTTCGTGTGAGCGTTG
GTATCGAGCACATTGACGATATCCGTGAAGATTTTGCGCAGGCGCTGGC
GACCGCGCTGGTTGATGCGGAGGGTGCGTAAGGATCCAGAGGTATATAT
TAATGCGTATCCAGCGTTACATTCTGATGACCGCGCTGATCAGCCAACC
GGACCTGCTGCCGCCGCCGGCGCCGGAGCGTTGCCCGCCGCAGCAAACC
GCGCGTCTGTTCCGTGAAACCCCGCTGCTGCTGGACTGCGGTCAGGTGG
TTCAAGATGTGCGTGTTGCGTACCACACCTATGGCACCCCGAGCGACCA
TGCGATCCTGGTGCTGCATGCGCTGACCGGCACCAGCGCGGTTCATGAG
TGGTGGCCGGATTTTCTGGGTGAAGGCAAGCCGCTGGACCCGACCCGTG
ATTATATTGTTTGCGCGAACGTTCTGGGTGGCTGCGCGGGTAGCACCGG
TCCGGCGGAGCTGCCGCGTGTGAACGGTGAAGACCCGCCGCTGACCCTG
CGTGATATGGCGCGTGTGGGTCGTGCGCTGCTGGAGGAACTGGGCGTTC
GTCGTGTGAGCGTTATTGGTGCGAGCATGGGTGGCATGCTGGCGTATGC
GTGGCTGCTGGAGTGCCCGGACCTGGTGGATCGTGCGGTTATCATTGGT
GCGCCGGCGCGTCACAGCCCGTGGGCGATTGGTCTGAACACCGCGGCGC
GTAACGCGATTCGTGCGGCGCCGGGTGGCGAGGGTCTGAAGGTTGCGCG
TCAGATCGCGATGCTGAGCTATCGTAGCCCGGAGAGCTTCGCGCTGACC
CAGAGCGGTTGGGGCACCCGTCGTCCGGGCACCCCGGACATTACCACCT
ACCTGGAGCACCAGGGTGAAAAACTGAGCACCCGTTTCTGCGAGCGTAG
CTATCTGGCGCTGACCGGCGCGATGGACCGTTTTCAACCGACCGATGCG
GAACTGCGTAGCATCCGTGTGCCGGTTCTGGTGGTTGGTATTAGCAGCG
ATGTGCTGTACCCGCCGGCGGAAGTGCGTACCTATGCGGGTCTGCTGCC
GCGTGGCCAGTACCTGGAACTGCAAAGCCCGCACGGCCATGATGCGTTC
CTGATCGATCCGCAGGGTCTGCCGGAAGCGGCGGCGGCGTTCCTGCACG
GTGCGTAACTCGAGCAACCTGGAGGCGGGCGCAGGCCCGCCTTTT.

In some embodiments, the bacterial cell comprises an exogenous nucleic acid molecule or a plurality thereof, comprising the sequence:

(SEQ ID NO: 31)
TTTATTCTTGACACTAGTCGGCCAAAATGATATAATACCTGAGTTTAAC
TTTAAGAGAGGTATATATTACCATGGGTAGCAAGAACTACCGTTTCGAG
ACCCTGCAAGTGCACGGTGGCCAAGAAGTTGACCCGACCACCAACAGCC
GTGCGGTGCCGATCTACCAGACCAGCAGCTATGTTTTTAACAGCGCGGA
GCATGGTGCGAACCTGTTCGCGCTGAAGGAATTTGGCAACATCTATACC
CGTATTATGAACCCGACCAGCGACGTTTTCGAGAAACGTATGGCGGCGC
TGGAAGGTGGCGTGGCGGCGGTTGCGACCGCGAGCGGTCAGGCGGCGCA
ATTCCTGGCGCTGAACAACTTTCTGAGCGTGGGCGATAACTTCGTTACC
AGCCCGTTTCTGTACGGTGGCAGCTATAACCAATTCAAAGTGAGCTTTA
AACGTATCGGTATTGAGGCGCGTTTTGCGAAGAGCGACAAAGTTGACGA
TCTGGCGGCGGAAATCAACGATAAGACCAAAGCGATCTACGTGGAGACC
ATTGGCAACCCGGAGTTCAACGTTCCGGACTTTGAAGCGATCGCGGCGC
TGGCGAAGAAACACGACATTCCGCTGGTTGTGGATAACACCTTCGGTGC
GGGTGGCTATCTGTGCCAGCCGATCAAGCACGGCGCGAACATTGTGACC
AGCAGCGCGACCAAATGGATCGGTGGTCACGGCACCAGCATTGGTGGCA
TCATTGTTGATGGTGGCAACTACAACTGGGGTAACGGCAAGTTCCCGCA
ATTTAGCGAGCCGAGCGAAGGTTATCACGGCCTGAACTTCTGGGAGACC
TTTGGTGACAACAACCCGCTGGGTCTGCCGAACATTGCGTTCGCGATTC
GTGCGCGTGTGGAAGGTCTGCGTGATTTTGGCCCGGCGATCAGCCCGTT
CAACAGCTTTCTGCTGCTGCAAGGTCTGGAGACCCTGAGCCTGCGTGTG
CAACGTACCGTTGACAACGCGCTGGAGCTGGCGAAATGGCTGGAAGCGC
ACCCGAAGGTGAAAAGCGTTAACTATCCGGGTCTGACCAACAGCCCGTA
CCACGCGACCGCGAAGAAATATCTGACCCACGGTTTCGGTGGCGTGCTG
AGCTTTGAGATTGAAGGCGATAAGGAGACCGCGAGCAACTTTATCAACA
ACCTGGAACTGATTAGCCACCTGGCGAACGTTGGTGACGCGAAAACCCT
GATCATTCAGCCGAGCGCGACCACCCACCAGCAACTGAGCGATGAAGCG
CAAATTGCGGCGGGTGTGACCCCGAGCCTGCTGCGTATTAGCAGCGGCA
TCGAGCACATTGAAGACCTGAAGGCGGATCTGACCGCGGCGTTCGATAA
AATCTAAGGATCCAGAGGTATATATTAATGAACCTGCAAAGCCCGCACC
TGACCATCGAGATGACCCAGGAAATTTTCTACTGCCAAGAGGCGCTGAG
CCTGGAGAGCGGCGAGAGCTTCCCGGAATTTCAACTGAGCTTTACCACC
CAGGGCCAACTGAACGCGAACAAGGACAACGTGATCTGGGTTCTGCACG
CGCTGACCGGTGATGCGAACCCGCACGAGTGGTGGAGCGGTCTGATCGG
CGAAGACAAGTTCTTTGATCCGAGCAAATATTTCATTGTGTGCGCGAAC
TTTCTGGGTAGCTGCTACGGCAGCACCCAGCCGCTGAGCAACAACCCGA
ACAACGGTAAACCGTACTATTACGACTTCCCGAACATCACCACCCGTGA
CATTGCGAGCGCGCTGGATAAGCTGCGTATCCACCTGGGCCTGGAGAAA
ATCAACACCGTGATTGGTGGCAGCCTGGGTGGCCAAGTGGGTCTGGAAT
GGGCGGTTAGCCTGGGCGAGAAGCTGGAAAACGCGATCATTGTTGCGAG
CAACGCGAAAGCGAGCCCGTGGATCATTGGTTTTAACGAGACCCAGCGT
ATGGCGATCGAAAGCGATAGCACCTGGGGCAAGACCCAACCGGAGGCGG
GTAAGAAAGGCCTGGAAACCGCGCGTGCGATTGGTATGCTGAGCTATCG
TCACCCGATGACCTTCCTGCAAAACCAAAGCGAGACCGAGGAAAAGCGT
GACGATTTTAAAATCAGCAGCTATCTGCGTTACCAGGGCCTGAAGCTGG
CGAACCGTTTCAACGCGATGAGCTACTGGATTCTGAGCAAAGCGATGGA
CAGCCACGATATTGGTCGTGGTCGTGGTGGCACCCCGGTGGCGCTGAGC
AACATCAAGTGCAAAGTGCTGAGCATCGGTGTTGACACCGATATTCTGT
TTACCAGCGAGGAAAGCCGTTATATTAGCAAGCACGTTCCGAAAGGCAC
CTATCGTGAGATCAGCAGCATTTACGGCCACGACGCGTTCCTGATCGAG
TATGAACAGCTGCAATACATTCTGAAGAGCTTCTACCTGGAAAACAACG
GCTAACTCGAGCAACCTGGAGGCGGGCGCAGGCCCGCCTTTT.

In some embodiments, the bacterial cell comprises an exogenous nucleic acid molecule or a plurality thereof, comprising the sequence:

(SEQ ID NO: 32)
TTTATTCTTGACACTAGTCGGCCAAAATGATATAATACCTGAGTTTAAC
TTTAAGAGAGGTATATATTACCATGGGTCCGAAGTACGACAACAGCAAC
GCGGATCAGTGGGGCTTCGAGACCCGTAGCATCCACGCGGGTCAGAGCG
TTGACGCGCAAACCAGCGCGCGTAACCTGCCGATTTACCAGAGCACCGC
GTTCGTGTTTGACAGCGCGGAGCACGCGAAACAACGTTTCGCGCTGGAA
GATCTGGGCCCGGTTTATAGCCGTCTGACCAACCCGACCGTGGAGGCGC
TGGAAAACCGTATTGCGAGCCTGGAGGGTGGCGTTCATGCGGTGGCGTT
TAGCAGCGGTCAGGCGGCGACCACCAACGCGATCCTGAACCTGGCGGGT
GCGGGTGACCACATTGTTACCAGCCCGCGTCTGTATGGTGGCACCGAAA
CCCTGTTCCTGATCACCCTGAACCGTCTGGGCATTGATGTTAGCTTTGT
GGAGAACCCGGATGATCCGGAAAGCTGGCAGGCGGCGGTTCAACCGAAC
ACCAAGGCGTTCTTTGGCGAGACCTTTGCGAACCCGCAAGCGGACGTGC
TGGATATCCCGGCGGTTGCGGAAGTGGCGCACCGTAACAGCGTTCCGCT
GATCATTGACAACACCATTGCGACCGCGGCGCTGGTGCGTCCGCTGGAG
CTGGGTGCGGATGTGGTTGTGGCGAGCCTGACCAAGTTCTACACCGGTA
ACGGCAGCGGTCTGGGTGGCGTTCTGATCGACGGTGGCAAATTTGATTG
GACCGTGGAAAAGGACGGCAAAAGCGTTTTCCCGTATTTTGTTACCCCG
GATGCGGCGTACCACGGTCTGAAGTATGCGGATCTGGGTGCGCCGGCGT
TTGGTCTGAAAGTTCGTGTGGGCCTGCTGCGTGACACCGGTAGCACCCT
GAGCGCGTTTAACGCGTGGGCGGCGGTTCAAGGCATCGATACCCTGAGC
CTGCGTCTGGAGCGTCACAACGAAAACGCGATTAAGGTGGCGGAGTTCC
TGAACAACCACGAGAAGGTTGAAAAAGTGAACTTTGCGGGTCTGAAGGA
TAGCCCGTGGTACGCGACCAAGGAAAAACTGGGCCTGAAATATACCGGT
AGCGTGCTGACCTTCGAGATCAAGGGTGGCAAAGACGAAGCGTGGGCGT
TTATTGATGCGCTGAAACTGCACAGCAACCTGGCGAACATCGGCGACGT
TCGTAGCCTGGTTGTGCATCCGGCGACCACCACCCATAGCCAAAGCGAT
GAGGCGGGCCTGGCGCGTGCGGGTGTGACCCAAAGCACCGTTCGTCTGA
GCGTGGGTATCGAGACCATTGACGATATCATTGCGGACCTGGAAGGTGG
CTTCGCGGCGATTTAAGGATCCAGAGGTATATATTAATGCCGACCCTGG
CGCCGAGCGGTCAGCTGGAGATCCAAGCGATTGGTGACGTTAGCACCGA
GGCGGGCGCGATCATTAAGAACGCGGAAATTGCGTACCACCGTTGGGGT
GAGTATCGTGTGGACAAAGAAGGCCGTAGCAACGTGGTTCTGATCGAAC
ACGCGCTGACCGGTGATAGCAACGCGGCGGACTGGTGGGCGGATCTGCT
GGGTCCGGGCAAGGCGATCAACACCGACATTTACTGCGTTATCTGCACC
AACGTGATCGGTGGCTGCAACGGCAGCACCGGTCCGGGCAGCATGCACC
CGGATGGTAACTTCTGGGGCAACCGTTTTCCGGCGACCAGCATTCGTGA
CCAGGTTAACGCGGAGAAACAATTCCTGGATGCGCTGGGTATTACCACC
GTTGCGGCGGTGCTGGGTGGCAGCATGGGTGGCGCGCGTACCCTGGAGT
GGGCGGCGATGTATCCGGAAATCGTTGGTGCGGCGGCGGTGCTGGCGGT
TAGCGCGCGTGCGAGCGCGTGGCAGATCGGCATTCAGAGCGCGCAAATC
AAGGCGATTGAAAACGATCACCACTGGCACGAGGGTAACTACTATGAAA
GCGGCTGCAACCCGGCGACCGGTCTGGGTGCGGCGCGTCGTATTGCGCA
CCTGACCTACCGTGGTGAGCTGGAAATCGACGAGCGTTTTGGCACCAAG
GCGCAGAAAAACGAAAACCCGCTGGGTCCGTATCGTAAGCCGGATCAAC
GTTTCGCGGTTGAGAGCTACCTGGACTATCAGGCGGATAAACTGGTTCA
ACGTTTTGACGCGGGTAGCTACGTGCTGCTGACCGATGCGCTGAACCGT
CACGACATTGGCCGTGATCGTGGTGGCCTGAACAAGGCGCTGGAGAGCA
TTAAAGTGCCGGTTCTGGTGGCGGGCGTTGACACCGATATCCTGTACCT
GTATCACCAGCAAGAACACCTGAGCCGTAACCTGGGTAACCTGCTGGCG
ATGGCGAAAATCGTTAGCCCGGTGGGTCATGATGCGTTCCTGACCGAAA
GCCGTCAAATGGATCGTATTGTGCGTAACTTCTTTAGCCTGATCAGCCC
AGGACGAGACAACCCGAGCACCTACATTGAATTTTATATCTAACTCGAG
CAACCTGGAGGCGGGCGCAGGCCCGCCTTTT.

In some embodiments, the bacterial cell comprises an exogenous nucleic acid molecule or a plurality thereof, comprising the sequence:

(SEQ ID NO: 33)
TTTATTCTTGACACTAGTCGGCCAAAATGATATAATACCTGAGTTTAAC
TTTAAGAGAGGTATATATTACCATGGGTCGTTTCGAGACCCTGCAACTG
CATGCGGGTTATGAGCCGGAACCGACCACCCTGAGCCGTCAAGTTCCGA
TCTACCCGACCACCAGCTATGTGTTTAAAAGCCCGGAGCATGCGGCGAA
CCTGTTCGCGCTGAAGGAATTTGGTAACATCTATAGCCGTATTATGAAC
CCGACCGTGGACGTTCTGGAGAAACGTCTGGCGGCGCTGGAAGGTGGCA
AAGCGGCGCTGGCGACCGCGAGCGGTCATGCGGCGCAGTTTCTGGCGCT
GACCACCCTGGCGCAAGCGGGCGATAACATCGTTAGCACCCCGAACCTG
TACGGTGGCACCTTCAACCAGTTTAAGGTTACCCTGAAACGTCTGGGCA
TTGAAGTGCGTTTCACCAGCCGTGAGGAACGTCCGGAGGAATTTCTGGC
GCTGACCGATGAGCGTACCCGTGCGTGGTGGGTGGAAAGCATCGGTAAC
CCGGCGCTGAACATTCCGGACCTGGAGGCGCTGGCGCAAGCGGCGCGTG
AAAAAGGTGTGGCGCTGATCGTTGATAACACCTTCGGCATGGGTGGCTA
TCTGCTGCGTCCGCTGGCGTGGGGCGCGGCGCTGGTTACCCACAGCCTG
ACCAAGTGGGTGGGTGGCCACGGTGCGGTTATTGCGGGTGCGATTGTGG
ATGGTGGCAGCTTTCCGTGGGAGGGTGGCCGTTACCCGCTGCTGACCGA
ACCGCAGCCGGGTTATCACGGCCTGCGTCTGACCGAGGCGTTCGGTGAA
CTGGCGTTTATTGTGAAAGCGCGTGTTGACGGCCTGCGTGATCAAGGTC
AAGCGCTGGGTCCGTTTGAGGCGTGGGTGGTTCTGCTGGGTATGGAAAC
CCTGAGCCTGCGTGCGGAGCGTCATGTGGAAAACACCCTGCATCTGGCG
CACTGGCTGCTGGAGCAGCCGCAAGTGGCGTGGGTTAACTATCCGGGTC
TGCCGCACCACCCGCACCACGACCGTGCGCAGAAGTATTTCAAGGGTAA
ACCGGGCGCGGTTCTGACCTTTGGCCTGAAGGGTGGCTACGAAGCGGCG
AAACGTTTTATCAGCCGTCTGAAGCTGATTAGCCACCTGGCGAACGTGG
GTGACACCCGTACCCTGGCGATTCACCCGGCGAGCACCACCCACAGCCA
ACTGAGCCCGGAGGAACAGGCGCAAGCGGGTGTTAGCCCGGAGATGGTG
CGTCTGAGCGTTGGCCTGGAGCACGTGGAAGATCTGAAGGCGGAGCTGA
AAGAAGCGCTGGCGTAAGGATCCAGAGGTATATATTAATGCGTATCCAG
CGTTACATTCTGATGAGCGAGATCGCGCTGGAAGCGTGGGGTGAGCATG
AAGCGCTGCTGCTGAAGCCGCCGCGTAGCCCGCTGAGCATCCCGCCGCC
GAAGCCGCGTACCGCGGTGCTGTTCCCGCGTCGTGAGGGTTTTTACACC
GAGCTGGGTGGTTACCTGCCGGAAGTGCGTCTGCGTTTCGAAACCTACG
GCACCCTGAGCCGTCGTCGTGATAACGCGGTGCTGGTTTTTCATGCGCT
GACCGGTAGCGCGCACCTGGCGGGCACCTATGATGAGGAAACCTTCCGT
AGCCTGAGCCCGCTGGAGCAGGCGTTTGGTCGTGAAGGTTGGTGGGATA
GCCTGGTGGGTCCGGGTCGTATCCTGGACCCGGCGCTGTACTATGTGGT
TAGCGCGAACCACCTGGGTAGCTGCTATGGTAGCACCGGTCCGCTGAGC
CTGGACCCGCACACCGGTCGTCCGTATGGTCGTGATTTCCCGCCGCTGA
CCATTCGTGATCTGGCGCGTGCGCAAGCGCGTCTGCTGGACCACCTGGG
TGTGGAGAAGGCGATCGTTATTGGTGGCAGCCTGGGTGGCATGGTGGCG
CTGGAGTTCGCGCTGATGTACCCGGAACGTGTTAAGAAACTGGTGGTTC
TGGCGGCGCCGGCGCGTCACGGCCCGTGGGCGCGTGCGTTTAACCACCT
GAGCCGTCAGGCGATCCTGCAAGATCCGGAATACCAGAAGGGTAACCCG
GCGCCGAAGGGTATGGCGCTGGCGCGTGGTATTGCGATGATGAGCTATC
GTGCGCCGGAAGGTTTTGAAGCGCGTTGGGGTGCGGAGCCGGAACTGGG
CGAGACCTACCTGGACTATCAAGGTGAAAAGTTCCTGCGTCGTTTTCAC
GCGGAGAGCTATCTGGTGCTGAGCCGTGCGATGGACACCCATGATGTGG
GTCGTGGTCGTGGTGGCGTTGAGGAAGCGCTGAAACGTCTGCGTGCGAT
CCCGAGCCTGTTTGTGGGCATTGACACCGATCTGCTGTACCCGGCGTGG
GAAGTTCGTCAGGCGGCGAAGGCGGCGGGTGCGCGTTATCGTGAGATCA
AAAGCCCGCACGGCCATGATGCGTTCCTGATCGAAACCGATCAAGTTGA
GGAAATTCTGGACGCGTTTCTGCCGTAACTCGAGCAACCTGGAGGCGGG
CGCAGGCCCGCCTTTT.

In some embodiments, the exogenous nucleic acid molecule or a plurality thereof is integrated into the genome of the bacterial cell. In other embodiments, the exogenous nucleic acid molecule or a plurality thereof is comprised within a plasmid or other expression vector. In some embodiments, an expression vector comprises a viral expression vector. In some embodiments, the plasmid or the expression vector further comprises one or more nucleic acid sequences encoding: a tag, a linker, or a combination thereof. In some embodiments, the tag may be suitable for protein isolation and/or purification. In some embodiments, the linker is a flexible linker.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), or poly-Adenine sequence. The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be a viral promoter.

Methods for gene expression in bacteria are well known in the art. An ordinary skilled in the art would know how to express the polypeptide of the invention, A large number of E. coli expression plasmids, or B. subtilis plasmids, are available for a wide variety of needs. A heterologous polypeptide can be expressed in the cytoplasm of the bacterial cell. Non-limiting examples for E. coli expression vectors are the pCCL, pGEX and the pET series of vectors. In some embodiments, the expression vector, or plurality thereof, comprises a sequence encoding an antibiotic for positive selection of the bacterial cell expressing it.

In some embodiments, the bacterial cell further comprises L-methionine exporter. In some embodiments, the L-methionine exporter is encoded by the yjeH gene. YjeH is a member of the amino acid efflux (AAE) family within the amino acid-polyamine-organocation (APC) superfamily of transporters. In some embodiments, yjeH encodes a L-methionine and branched chain amino acid (BCAA) efflux transporter in E. coli. In some embodiments, yjeH is overexpressed in the bacterial cell disclosed herein. In some embodiments, a cell as disclosed herein, characterized by overexpression of yjeH comprises one or more copies of the yjeH gene. In some embodiments, one or more copies of the yjeH gene are located within the genome of a cell as disclosed herein, extra-genomically, e.g., plasmid, etc., or both. In some embodiments, overexpression of yjeH results in increased tolerance to L-methionine. In some embodiments, overexpression of yjeH is associated with increased export of methionine and the BCAAs when the cell is grown in the presence of at least one of: Met-Met, Ile-Ile, Leu-Leu or Val-Val dipeptides. In some embodiments, absence or reduced expression of yjeH results in increased levels of intracellular methionine. In some embodiments, expression of yjeH is not regulated by the transcriptional repressor, metJ.

In some embodiments, there is provided a composition comprising the bacterial cell disclosed herein and a biologically accepted carrier.

As used herein, the term “carrier,” refers to any component of a composition that is not the active agent. In some embodiments, the carrier comprises a culture liquid medium. In some embodiments, the carrier comprises a solid medium (e.g., agar).

In some embodiments, there is provided a method for producing an amino acid.

In some embodiments, the method comprises: (a) providing a bacterial cell comprising: (i) an exogenous nucleic acid molecule or a plurality thereof encoding metX and metY, and (ii) inactive metA and metB genes; and, (b) culturing the bacterial cell from step (a) such that the metX and metY encoded by the exogenous nucleic acid molecule are expressed, thereby, producing the amino acid.

In some embodiments, culturing is in a culture or a growth medium. Types of culture media are common and would be apparent to one of ordinary skill in the art of microbiology.

In some embodiments, the amino acid comprises an essential amino acid.

In some embodiments, the essential amino acid is selected from the group consisting of: alanine, valine, leucine, threonine, isoleucine, homoserine, methionine, aspartate, phenylalanine, glutamate, and any combination thereof.

In some embodiments, the amino acid comprises methionine. In some embodiments, the amino acid comprises or consists of L-methionine.

In some embodiments, there is provided a method for producing L-methionine.

In some embodiments, the method is for increasing production of an amino acid. In some embodiments, increasing it compared to a control cell. In some embodiments, a control cell comprises active metA, active metB, or both. In some embodiments, a control cell is devoid of metX, met Y, or both. In some embodiments, a control cell comprises an active metJ. In some embodiments, a control cell comprises active metA, active metB, active metJ and is devoid of metX and metY. In some embodiments, a control cell comprises a bacterial cell. In some embodiments, a control cell comprises an E. coli cell. In some embodiments, a control cell comprises an E. coli cell comprising active metA, active metB, active metJ and being devoid of metX and metY.

In some embodiments, the produced levels of an amino acid being produced by a bacterial cell as disclosed herein are at least 1.5-fold, at least 2-fold, at least 5-fold, at least 10-fold, or at least 20-fold greater than levels of amino acid being produced by a control bacterial cell, or any value and range therebetween. Each possibility represents a separate embodiment of the invention.

In some embodiments, the produced levels of an amino acid being produced by a bacterial cell as disclosed herein are 1.5-fold to 20-fold, 5-fold to 50-fold, 1.5-fold to 100-fold, 2-fold to 80-fold, 4-fold to 30-fold, 10-fold to 100-fold, 5-fold to 200-fold greater than levels of amino acid being produced by a control bacterial cell. Each possibility represents a separate embodiment of the invention.

In some embodiments, the produced levels comprises intracellular levels, extracellular levels, or both. In some embodiments, the produced levels are determined intracellularly, e.g., from an intracellular fraction of the bacterial cell, extracellularly, e.g., from an extracellular fraction wherein the bacterial cell is cultured, or both.

In some embodiments, the method further comprises a determining step, comprising determining the intracellular production level of the amino acid, the extracellular production level of the amino acid, or both.

In some embodiments, the method further comprises extracting the intracellular content of the bacterial cell. Methods and means for extracting bacterial cells are common and would be apparent to one of ordinary skill in the art.

In some embodiments, the method further comprises collecting or obtaining the cultured medium wherein the bacterial cell disclosed herein is cultured. In some embodiments, the collected culture medium comprises an extracellular fraction produced and/or secreted from the bacterial cell disclosed herein. In some embodiments, the method further comprises extracting the collected culture medium.

In some embodiments, there is provided an extract of the bacterial cell disclosed herein. In some embodiments, the extract comprises an amino acid. In some embodiments, the extract comprises L-methionine.

In some embodiments, the extract comprises intracellular produce, extracellular produce, or both, of the bacterial cell disclosed herein

In some embodiments, there is provided a method for producing L-methionine, the method comprising: (a) providing a bacterial cell comprising: (i) an exogenous nucleic acid molecule or a plurality thereof encoding metX and metY, and (ii) inactive metA and metB genes; and (b) culturing the bacterial cell from step (a) such that metX and metY encoded by the exogenous nucleic acid molecule are expressed, thereby, producing L-methionine.

In some embodiments, the method comprises: (a) providing a bacterial cell comprising: (i) an exogenous nucleic acid molecule or a plurality thereof encoding at least one of: SEQ ID NOs: 1-6, and 50, and SEQ ID NOs: 7-12, and (ii) inactive metA and metB genes; and, (b) culturing the bacterial cell from step (a) such that at least one of: SEQ ID NOs: 1-6, and 50, and at least one of: SEQ ID NOs: 7-12, are expressed and/or translated.

In some embodiments, the method comprises: (a) providing a bacterial cell comprising: (i) an exogenous nucleic acid molecule or a plurality thereof encoding at least one of: SEQ ID NOS: 1-6, and 50, and SEQ ID NOs: 7-12, and (ii) inactive metA, metB, and metJ genes; and, (b) culturing the bacterial cell from step (a) such that at least one of: SEQ ID NOs: 1-6, and 50, and at least one of: SEQ ID NOs: 7-12, is expressed, e.g., transcribed and/or translated.

In some embodiments, the method comprises: (a) providing a bacterial cell comprising: (i) an exogenous nucleic acid molecule or a plurality thereof encoding at least one of: SEQ ID NOS: 1-6, and 50, and SEQ ID NOs: 7-12, (ii) inactive metA, metB, and metJ genes, and (iii) one or more copies of yjeH gene; and, (b) culturing the bacterial cell from step (a) such that at least one of: SEQ ID NOs: 1-6, and 50, and at least one of: SEQ ID NOs: 7-12, is expressed, e.g., transcribed and/or translated.

In some embodiments, the method comprises: (a) providing a bacterial cell comprising: (i) an exogenous nucleic acid molecule or a plurality thereof encoding at least one of: SEQ ID NO: 3 or 5, and SEQ ID NOs: 9 or 11, (ii) inactive metA, metB, and metJ genes, and (iii) one or more copies of the yjeH gene; and, (b) culturing the bacterial cell from step (a) such that at least one of: SEQ ID NOs: 3 or 5, and at least one of: SEQ ID NOs: 9 or 11, is expressed, e.g., transcribed and/or translated.

In some embodiments, the bacterial cell is cultured in media comprising MOPS (3-(N-morpholino) propanesulfonic acid). In some embodiments, the media comprises an effective amount of a sulfur compound. Examples for sulfur compounds that can be provided in the media are K₂SO₄ and FeSO₄.

In some embodiments, transformed cells are cultured under effective conditions, which allow for the expression of high amounts of recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals, and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH, and oxygen content appropriate for a recombinant cell. In some embodiments, culturing conditions are within the expertise of one of ordinary skill in the art.

A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

As used herein, the term “encoding” refers to molecule comprising a DNA sequence which can be transcribed into an RNA sequence which can be translated into the encoded protein or a molecule comprising the RNA sequence which can be translated into the encoded protein. In some embodiments, the molecule is a DNA molecule. In some embodiments, the molecule is an RNA molecule. In some embodiments, the DNA is cDNA. In some embodiments, the molecule is a DNA/RNA hybrid. In some embodiments, the molecule comprises non-naturally occurring nucleotides.

Expressing of a polynucleotide within a cell is well known to one skilled in the art. It can be carried out by, among many methods, transfection, viral infection, or direct alteration of the cell's genome.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

### General

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polynucleotides and polypeptides, size, weight, or length, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one”, are used interchangeably in this application.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by references into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

Generally, the nomenclature used herein and the laboratory procedures utilized in the present invention include immunological, chemical, molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Material and Methods

### Bacterial Strains and Growth Conditions

E. coli MG1655 strain was used in this study. Bacteria were routinely grown in Luria-Bertani (LB) media at 37° C. For screening of the enzymatic variants bacteria were grown in liquid or solid (supplemented with 1.5% agar) MOPS media (8.73 g/L MOPS, 0.71 g/L tricine, 0.51 g/L NH4Cl, 0.05 g/L K₂SO₄, 2.92 g/L NaCl, 2.8 mg/L FeSO₄, 0.074 mg/L, 0.1 g/L MgCl2 1 ml/L trace elements, K2HPO4 30 mg/L, Glucose 2 g/L, pH 7.3).

### Generation of Methionine Auxotrophic Mutants

Target deletion mutations were generated in MG1655 by the lambda red recombinase procedure (Datsenko and Wanner, 2000) using primers listed in Table 1, with pkD4 plasmid carrying KnR cassette serving as a template for PCR reactions. Mutations were verified using nearby locus-specific primers (Table 1) with the respective primers k2 or kt (Datsenko and Wanner, 2000). Afterwards the cassette was removed, and double/triple mutations were generated in single/double mutant strains as described by Datsenko and Wanner (2000). The mutations were generated by following scheme ΔmetA→ΔmetAB→ΔmetABJ.

TABLE 1
Primers used in this study

| Primer name | Primer sequence | Description |
|---|---|---|
| MetA-D-For | CGAGCTACCCGCCGTCAATTTCTT | Generation of AmetA |
| | GCGTGAAGAAAACGTCTTTGTGAT | mutation |
| | GACATATGAATATCCTCCTTAG (SEQ | |
| | ID NO: 36) | |
| MetA-D-Rev | TGTGCCGTAGATCGTATGGCGTG | Generation of AmetA |
| | ATCTGGTAGACGTAATAGTTGAGC | mutation |
| | CAGTGTAGGCTGGAGCTGCTTCG | |
| | (SEQ ID NO: 37) | |
| MetB-D-For | ACAGGCCACCATCGCAGTGCGTAGC | Generation of AmetB mutation |
| | GGGTTAAATGACGACGAACAGTATG | |
| | CATATGAATATCCTCCTTAG (SEQ ID | |
| | NO: 38) | |
| MetB-D-Rev | CGGAAGCCATTTTCCAGGTCGGCAA | Generation of AmetB mutation |
| | TTAAATCTTCGCCATCTTCAATACCT | |
| | GTAGGCTGGAGCTGCTTCG (SEQ ID | |
| | NO: 39) | |
| MetJ-D-For | GGAGCGGCGAATATATCAGCCCA | Generation of AmetJ mutation |
| | TACGCTGAGCACGGCAAGAAGAG | |
| | TGAATGTAGGCTGGAGCTGCTTCG | |
| | (SEQ ID NO: 40) | |
| MetJ-D-Rev | CACGTCTCCGGGTTAATCCCCATC | Generation of AmetJ mutation |
| | TCACGCATGATCTCTTTTGCCGCT | |
| | TCCATATGAATATCCTCCTTAG (SEQ | |
| | ID NO: 41) | |
| MetA-For | GTTATGCCGATTCGTGTGCC (SEQ ID | Verification of AmetA |
| | NO: 42) | mutation |
| MetA-Rev | AGGTAAGGTGCTGAATCGCT (SEQ | Verification of AmetA |
| | ID NO: 43) | mutation |
| MetB-For | ACATTTCACCGACAAAGCCC (SEQ ID | Verification of AmetB |
| | NO: 44) | mutation |
| MetB-Rev | TTTACCCCTTGTTTGCAGCC (SEQ ID | Verification of AmetB |
| | NO: 45) | mutation |
| MetJ-For | ACTGTGTGGTCTGGTCTCAA (SEQ ID | Verification of AmetJ |
| | NO: 46) | mutation |
| MetJ-Rev | CTGATAAGCGTAGCGCATCA (SEQ | Verification of AmetJ |
| | ID NO: 47) | mutation |
| yjeH-For | GCGCCATGGATGAGTGGACTCAAAC | Amplification of genomic |
| | AAGAAC (SEQ ID NO: 48) | copy of yjeH and addition of a |
| | | NcoI site for cloning |
| | | (underlined) |
| yjeH-Rev | GCGCTCGAGTTATGTGGTTATGCCA | Amplification of genomic |
| | TTTTCC (SEQ ID NO: 49) | copy of yjeH and addition of a |
| | | XhoI site for cloning |
| | | (underlined) |

Underlined sequences refer to sequences derived from pKD4 plasmid (Datsenko and
Wanner, 2000)

Complementation with MetXY Constructs and Activity Validation

pCCI plasmid carrying metXY synthetic operons were transformed to electrocompetent ΔmetAB/ΔmetABJ mutants. Following transformation, several colonies growing on LB agar plates supplemented with 30 μg/ml chloramphenicol, were tested for the presence of the correct plasmid by colony PCR with M13F and M13R primers. Positive clones were further screened for the growth pattern in methionine-depleted minimal media (MOPS). Briefly, Cultures were prepared by inoculation of a single colony grown on LB into 5 ml MOPS media and incubated overnight at 37° C., 200 rpm. The culture was diluted 1000× fold in fresh MOPS media and 200 μl were placed in each well of 96-well plate (Costar). Bacteria were grown for 20 h at 37° C. (constant orbital shaking 280 rpm) through interval measuring of OD (600 nm) every 16 minutes using Infinite M200 Plate Reader (Tecan). Each sample was tested in triplicates. For control, MOPS media was supplemented with 5-50 μg/ml of methionine (Merck) or 0.1-0.5 mg/ml of norleucine (Merck). Alternatively, a single colony grown on Luria-Bertani (LB) media was spread on MOPS solid media and visual growth following 24-72 h at 37° C. was detected.

Construction of a yjeH Overexpression Plasmid

The yjeH gene was amplified from genomic DNA extracted from E. coli MG1655 using a forward primer that adds an NcoI restriction site (GCGCCATGGATGAGTGGACTCAAACAAGAAC; SEQ ID NO: 48), and a reverse primer adding an XhoI restriction site GCGCTCGAGTTATGTGGTTATGCCATTTTCC; SEQ ID NO: 49). The purified PCR product was digested with NcoI and XhoI and inserted into pTrcHis-a digested with the same enzymes. The correct construct was validated by sequencing.

Table 2 summarizes the terminology and the strains that were used in the study.

TABLE 2
Summary of terminology and strains

| Name | Description |
|---|---|
| Wildtype, WT | E. coli MG1655 |
| ΔmetAB | E. coli MG1655 with a deletion of the metA and metB genes |
| ΔmetABJ | E. coli MG1655 with a deletion of the metA, metB and metJ genes |
| ΔmetABJ-Y | E. coli MG1655 with a deletion of the metA, metB and metJ genes |
| | overexpressing the methionine exporter YjeH |
| DG/CM/LI/CG | Complementation with a pCCI plasmid expressing metA and metB |
| | of the noted bacterial strain |

### Qualitative Evaluation of Extracellular Methionine Levels

For qualitative analysis of methionine concentration in the media, bacteria were grown over-night at 37° C. in 5 ml of MOPS media and filtered through a 0.22 membrane to remove bacterial cells. The filtered media was diluted two-fold in fresh MOPS media and the growth of ΔmetAB mutant in pre-cultured media was determined by OD (600 nm) measurement as described.

Methionine Extraction from Lysate and Medium to Evaluate Intra- and Extra-Cellular Methionine Levels

To evaluate the intra-cellular level of methionine, amino acids were extracted from 1 ml bacteria pellet using methanol/water/chloroform at ratio of 1:1:2.5 respectively. After centrifugation, the crude extract was separated to polar and non-polar phase with the addition of 300 μl water and 300 μl chloroform and centrifuge for 10 min. Four hundred (400) μl from the top polar phase were vacuum dried. In addition, to evaluate extra-cellular methionine level, amino acids were extracted from 500 μl of the growth medium by adding 500 μl of chloroform and centrifuge for 10 min. Four hundred (400) μl from the upper polar phase was vacuum dried. The vacuum-dried fraction was dissolved in 40 μl of 20 mg of methoxyamine hydrochloride in 1 ml pyridine and incubated at 37° C. for 2 h with vigorous shaking, followed by derivatization for 30 min in N-methyl-N(trimethylsilyl)-trifluoroacetamide at 37° C. One μl of the sample was injected into a BP5MS capillary column (SEG; 30 m, 0.25-mm i.d., and 0.25-mm thickness).

### Evaluation of Methionine Levels by GC-MS

All analyses were carried out on a GC-MS system (Agilent 7890A) coupled with a mass selective detector (Agilent 5975c) and a Gerstel multipurpose sampler (MPS2). For free amino acid detection, the single-ion mass method was used. Amino acid standards of 5, 10, 25, 50, 100, and 200 μM were used to generate standard calibration curves, and ribitol (2 mg in 1 ml HPLC grade water) was used as internal standards. The peak areas were calculated from the standard calibration curves and normalized to the ribitol signal.

### Example 1

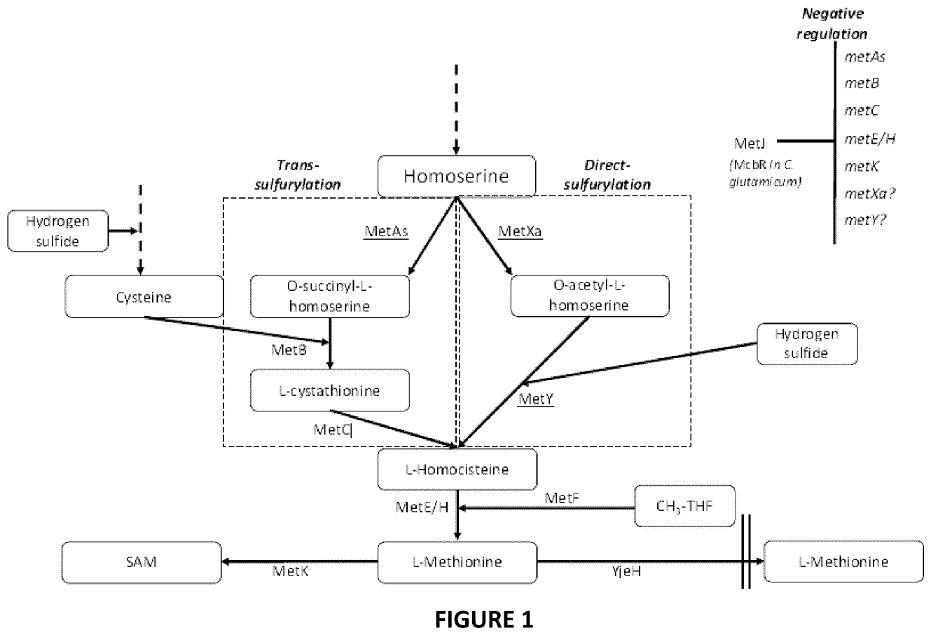

Engineering of Methionine Auxotroph E. coli

Whereas most microorganisms rely on sulfur assimilation via the direct-sulfurylation methionine biosynthesis pathway, several bacteria, such as Escherichia coli (E. coli), utilizes the less efficient and highly regulated trans-sulfurylation pathway. In the trans-sulfurylation pathway in E. coli, homoserine is converted to L-homocysteine in three steps catalyzed by enzymes encoded by metA, metB and metC genes. Sulfur is assimilated into the intermediate O-succinyl or O-acetyl L-homoserine from cysteine by an enzyme encoded by metB to form cystathionine. The enzyme encoded by metC is a lyase converting cystathionine to L-homocysteine. In the direct sulfurylation pathway, L-homoserine is converted to L-homocysteine in two steps catalyzed by the enzymes encoded by metX and metY. Sulfur in the form of hydrogen sulfide is assimilated into O-acetyl L-homoserine by the enzyme encoded by metY to directly form L-homocysteine. FIG. 1 illustrates the two orthogonal pathways.

The trans-sulfurylation pathway is less parsimonious in terms of step numbers and is dependent on the type of the sulfur donor molecule. While E. coli is a valuable work-horse in synthetic biology, the trans-sulfurylation pathway is a limiting step in the bioproduction of methionine.

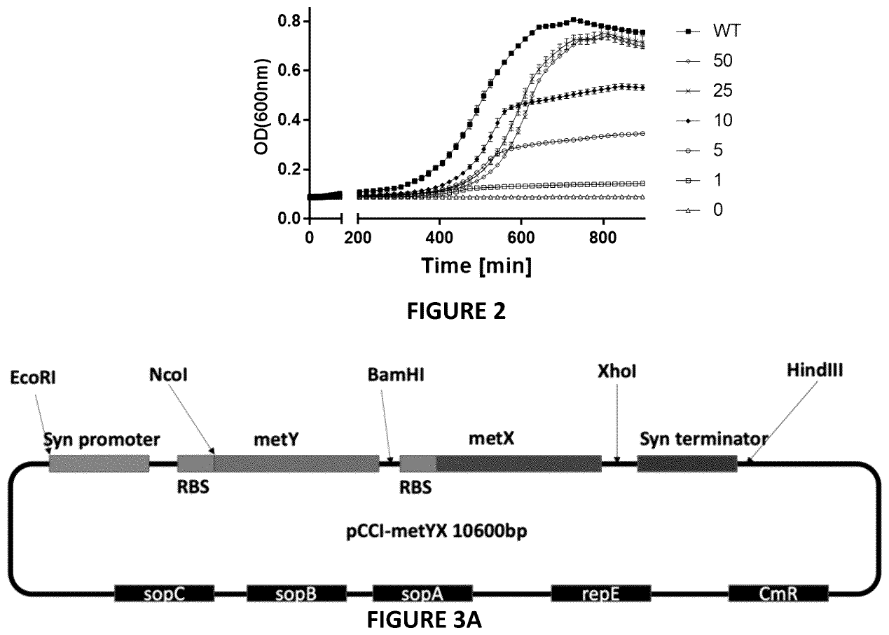

To exploit the option of converting the methionine biosynthesis pathway from trans—to direct-sulfurylation, the inventors deleted the two essential genes in the methionine pathway of E. coli, metA and metB, encoding the enzymes HST and CgS, respectively. The deletion generated a methionine auxotroph E. coli strain (ΔmetAB). FIG. 2 shows growth curves of the ΔmetAB in MOPS minimal media containing glucose and ammonium chloride as the sole carbon and nitrogen source, respectively, and K₂SO₄ as the sulfur source. To test the effect of methionine on the growth rate, variable concentrations of external methionine were supplemented as indicated (FIG. 2, legend in μM). While a normal growth rate is observed for the wild-type bacteria, the ΔmetAB auxotroph is incapable of growing without supplementation of methionine. However, the addition of methionine rescues bacterial growth, reaching maximal growth and cell density resembling the WT bacteria at a concentration of 25 μM and showing that indeed methionine is the limiting growth factor.

### Example 2

Complementation of E. coli ΔMetAB by MetX and MetY Genes of Various Bacterial Genomes

Aiming to explore the ability of E. coli to synthesize methionine via the direct-sulfurylation enzymes metX and metY, the inventors cloned four metY/X pairs of genes to complement the ΔMetAB auxotroph bacteria. It was expected that such complementation would enable E. coli to grow without supplementation of external methionine. The inventors applied two complemented selection criteria. The first was metX enzymes showing a range of catalytic activity of 10³-10⁴ nmol·min⁻¹·mg⁻¹. The second criteria was that the relevant bacterial genome for the sequence of the counterpart metY gene is known. Based on this rationale, the inventors selected the metY/X pairs from the following four strains: i) Corynebacterium glutamicum (CG), ii) Leptospira interrogans (LI), iii) Cyclobacterium marinum (CM) and iv) Deinococcus geothermalis (DG). Interestingly, the sequence identity between the 4 selected metX genes ranged from 33.5% to 37% and between the 4 selected metY genes from 43.9% to 56.7%. Since the arrangement of the genes in each of the selected bacteria is different and in order to design a uniform construct, the metY/X genes were cloned into a pCC1 plasmid. The latter enabled to maintain the inserted genes at low to a single copy, similar to genomic sequences of the corresponding genes. The genes were codon-optimized for E. coli expression and synthesized and cloned with a preceding ribosomal binding site (RBS) to the plasmid. FIG. 3A shows a schematic illustration of the operon that was constructed. The ability of the complemented ΔmetAB to grow on liquid minimal media was then tested. The ΔMetAB bacteria cloned with the metY/X of DG and CM (ΔMetAB-DG and ΔMetAB-CM in FIG. 3B), successfully complemented the auxotroph bacteria and after about 800 min reached the same growth rate and similar order of cell density as the WT. The complemented bacteria having metY/X of LI and CG (ΔMetAB-LI and ΔMetAB-CG in FIG. 3B) did not grow under these conditions. The ability of these complemented bacteria to grow on minimal media agar plates was also tested (FIG. 3C) and a similar pattern was observed in both assays. The ability of the CM and DG strains to grow on a minimal medium indicates that they can produce methionine at a level sufficient to maintain their growth. To quantify intracellular and extracellular methionine levels in the complemented bacteria, the methionine levels were evaluated using GC-MS and compared to those of the WT bacteria. The ΔmetAB-DG and ΔmetAB-CM strains exhibited a five-fold enhancement of intracellular methionine levels compared to WT (FIG. 3D, left). Analysis of the extracellular methionine in the growth medium indicated that ΔmetAB-DG exhibited significantly enhanced accumulation of extracellular methionine as compared to WT (18-fold). Although the difference in methionine accumulation was not significant, the ΔmetAB-CM also showed a five-fold increase compared to the WT (FIG. 3D, right).

### Example 3

Evaluation of Intra- and Extra-Cellular Methionine of Engineered E. coli with Deletion of metJ and Over-Expression of the Transporter YjeH

Having the transgenic E. coli strains that expressed the metY/X of DG and CM in ΔmetAB, the inventors further explored the effect of additional genetic variations related to the methionine biosynthetic pathway. To this end, the metJ repressor that is known to strictly regulate the transcription of multiple genes in the pathway (FIG. 1) was deleted. The E. coli methionine exporter protein YjeH that was also shown to have a strong positive effect on extracellular methionine accumulation was cloned to facilitate overexpression and enable methionine export. To evaluate the ability of the engineered strains to produce and export methionine, the bacteria were cultured in minimal media until reaching OD_{(600 nm)}=2.5. The intracellular and extracellular levels of methionine were evaluated using GC-MS (FIG. 4). By deleting MetJ at WT background, the level of intracellular and extracellular methionine increased by 15 and 161, respectively, for the ΔMetABJ-Y-CM, and by 10 and 127, respectively, for the ΔMetABJ-Y-DG, relative to wild-type (FIG. 4).

### Example 4

Validation of the Bioavailability of Extracellular Methionine Secreted from the Engineered DG and CM Strains

To confirm via an orthogonal approach the level and bioavailability of the extracellular methionine secreted from each engineered strain, the inventors supplemented fresh minimal media with medium at the end of the bacterial growth. Since no methionine exist in the fresh solution, the presence of methionine in the reused medium should reflect on the ability of the methionine-auxotroph E. coli ΔmetAB bacteria to grow. To this end, the used media was sterilized by passing it through a 0.22 μm membrane filter and added into a fresh minimal media in a 1:1 ratio. FIG. 5 shows the growth curves of the auxotroph ΔmetAB cells in each of the conditions (i.e., ΔmetABJ-Y-DG legend-growth curve of auxotroh ΔmetAB cells in reused medium mixture after the ΔmetABJ-Y-DG growth). The highest cell density is observed when the methionine-auxotroph cells were cultured when supplemented with the used medium of the ΔmetABJ-Y-CM/DG, suggesting that the highest methionine level was in it. Owing to the absence of methionine in the medium, no growth was observed in the used medium of the WT or the auxotroph ΔmetABJ. Both results are incongruent with the methionine levels that were measured for these strains in the GC-MS (FIG. 4).

### Example 5

metX and metY of DG Completements Methionine Secretion in Various Mutants

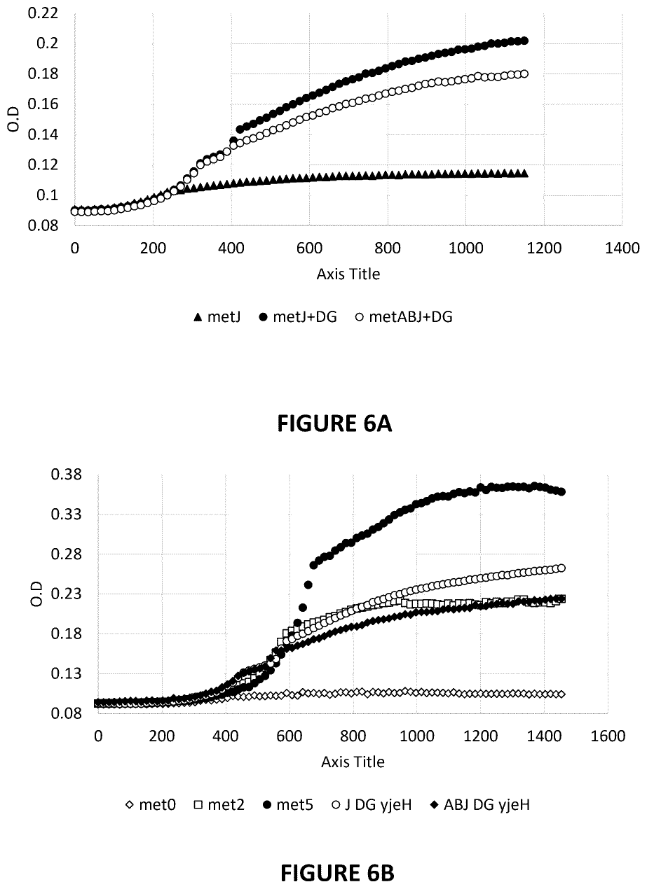

In the presence of exogeneous metY and metX, methionine secretion decreases following the deletion of metABJ, compared to the deletion of metJ alone (FIG. 6A). This trend is maintained following overexpression of yjeH (16 vs 11 μg/ml and 24 vs 16 μg/ml respectively; FIG. 6B)

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method of producing an amino acid, the method comprising:
(a) providing a bacterial cell comprising an exogenous nucleic acid molecule or a plurality thereof encoding metX, metY, and an amino acid transporter protein; and
(b) culturing said bacterial cell from step (a) such that said metX, metY, and amino acid transporter protein encoded by said exogenous nucleic acid molecule are expressed,
thereby, producing the amino acid.

2. The method of claim 1, wherein said bacterial cell further comprises at least one inactive: metA gene, metB gene, metJ gene, and any combination thereof.

3. (canceled)

4. The method of claim 1, wherein said exporter protein is L-methionine and BCAA exporter.

5. The method of claim 1, wherein said amino acid is an essential amino acid, optionally wherein said essential amino acid is selected from the group consisting of: methionine, alanine, valine, leucine, threonine, isoleucine, glutamate, and any combination thereof, and optionally wherein said methionine is L-methionine.

6-7. (canceled)

8. The method of claim 1, wherein said culturing comprises supplementing said bacterial cell with a culture medium comprising an effective amount of a sulfur compound, and optionally wherein the produced levels of said L-methionine being produced by said bacterial cell are at least 5-fold greater than levels of L-methionine being produced by a control bacterial cell.

9. (canceled)

10. The method of claim 1, wherein said produced levels of said amino acid comprises intracellular levels, extracellular levels, or both, of said amino acid.

11. A bacterial cell comprising: (i) an exogenous nucleic acid molecule or a plurality thereof, encoding homoserine O-acetyltransferase (metX) and O-acetylhomoserine sulfhydrylase (metY), and any one of: (ii) inactive homoserine O-succinyltransferase (metA) and cystathionine gamma-synthase (metB) genes; (iii) an inactive metJ methionine repressor gene; or both (ii) and (iii), and wherein said bacterial cell further comprising one or more inhibitory nucleic acid sequences being complementary to any one of: said metA, metB, and metJ genes.

12. The bacterial cell of claim 11, wherein any one of: said inactive genes is knocked out, knocked down, mutated, or chemically inhibited, and optionally wherein said inactive gene being mutated comprises a nucleic acid sequence comprising a premature stop codon compared to a wildtype form of said gene.

13-14. (canceled)

15. The bacterial cell of claim 11, wherein said encoded metX, comprises the amino acid sequence set forth in any one of SEQ ID Nos: 1-6, and 50, and optionally wherein said encoded metX, comprises the amino acid sequence set forth in any one of SEQ ID Nos: 3 and 5.

16. (canceled)

17. The bacterial cell of claim 11, wherein said encoded metY comprises the amino acid sequence set forth in any one of SEQ ID Nos: 7-12, and optionally wherein said encoded metY comprises the amino acid sequence set forth in any one of SEQ ID Nos: 9 and 11.

18. (canceled)

19. The bacterial cell of any one of claim 11, wherein said exogenous nucleic acid molecule or a plurality thereof, comprises a nucleic acid sequence being codon optimized for expression in said bacterial cell.

20. The bacterial cell of claim 11, being an Escherichia coli cell.

21. The bacterial cell of claim 11, wherein said exogenous nucleic acid molecule encoding said metX comprises the nucleotide sequence set forth in any one of SEQ ID Nos: 13-18.

22. The bacterial cell of claim 11, wherein said exogenous nucleic acid molecule encoding said metY comprises the nucleotide sequence set forth in any one of SEQ ID Nos: 19-24.

23. The bacterial cell of claim 11, wherein said encoded metX, comprises the amino acid sequence set forth in any one of SEQ ID Nos: 3 and 5, and said encoded metY comprises the amino acid sequence set forth in any one of SEQ ID Nos: 9 and 11.

24. The bacterial cell of claim 11, wherein said exogenous nucleic acid molecule encoding said metX comprises the nucleotide sequence set forth in any one of SEQ ID Nos: 15 and 16 and said exogenous nucleic acid molecule encoding said metY comprises the nucleotide sequence set forth in any one of SEQ ID Nos: 21 and 22.

25. The bacterial cell of claim 11, wherein said exogenous nucleic acid molecule or plurality thereof is: (i) integrated into the genome of said bacterial cell; or (ii) comprised within a plasmid or an expression vector, and optionally wherein encoding said metX and said metY are operably linked.

26. (canceled)

27. The bacterial cell of claim 11, wherein said exogenous nucleic acid molecule or a plurality thereof comprises the nucleic acid sequence set forth in any one of SEQ ID Nos: 25-30.

28. The bacterial cell of claim 11, further characterized by expression of the yjeH gene, and optionally wherein said bacterial cell comprises an L-methionine and branched chain amino acid (BCAA) exporter protein.

29. (canceled)

30. A composition comprising the bacterial cell of claim 11, and a biologically accepted carrier.
